(19) United States
(12) Patent Application Publication
(10) Pub. No.: US 2025/0118432 A1
(43) Pub. Date: Apr. 10, 2025

(54) WELL-BEING PLATFORM UTILIZING MACHINE LEARNING

(71) Applicant: Lotic.ai, Inc., Bee Cave, TX (US)

(72) Inventors: William WELSER, IV, Austin, TX (US); Jonathan COHEN, Ashland, OR (US)

(21) Appl. No.: 18/597,656

(22) Filed: Mar. 6, 2024

Related U.S. Application Data
(63) Continuation of application No. PCT/US24/18548, filed on Mar. 5, 2024.
(60) Provisional application No. 63/488,856, filed on Mar. 7, 2023.
(60) Provisional application No. 63/505,809, filed on Jun. 2, 2023.
(60) Provisional application No. 63/588,075, filed on Oct. 5, 2023.

(51) Int. Cl. G16H 50/20; G16H 20/70

(52) U.S. Cl. CPC G16H 50/20; G16H 20/70

(57) ABSTRACT

Described are methods, platforms, systems, and media, for enhancing well-being by using methodology including receiving media comprising an unstructured user-generated narrative; applying one or more algorithms to the unstructured user-generated narrative to extract semi-structured user context data; applying a first machine learning model to classify one or more of sentiment, intent, habits, patterns, beliefs, and motivations from at least the user context data, wherein the first machine learning model comprises an unsupervised machine learning model; applying a second machine learning model to identify one or more recommended next actions from at least the user context data, wherein the second machine learning model comprises a supervised machine learning model; and generating one or more well-being-related insights for the user based at least on the user context data, and one or more of the sentiment, intent, habits, patterns, beliefs, motivations, and one or more recommended next actions to encourage a healthy lifestyle.

### CROSS-REFERENCE

This application is a continuation application of PCT International Application No. PCT/US2024/018548, filed Mar. 5, 2024, which claims the benefit of U.S. Provisional Application No. 63/488,856, filed Mar. 7, 2023, U.S. Provisional Application No. 63/505,809, filed Jun. 2, 2023, and U.S. Provisional Application No. 63/588,075, filed Oct. 5, 2023, each of which is hereby incorporated by reference in its entirety herein.

### BACKGROUND

The well-being of an individual can include all aspects of a person, including physical, emotional, and mental aspects. The well-being of an individual can be affected by a variety of factors, such as past and/or ongoing events, stress, diet, lack of motivation, amount and/or type of physical activity, amount and/or quality of sleep, etc. There is ongoing research to understand how to better improve the well-being of individuals by identifying the factors that affect the individual.

### SUMMARY

Often the factors that affect the well-being of an individual are not readily apparent to the individual. Present systems and methods lack the ability of efficiently and accurately identifying the factors that affect the well-being of a person. Moreover, well-being, and the factors that affect well-being, change and evolve over time. The existing systems and methods are outdated, lack coordinated data and information, and often are removed from community engagement. This creates the need for an individual to often re-explain their symptoms and history to different people over the course of months or years without making progress in improving the well-being of that individual. Barriers can exist with how the individual communicates their thoughts or past and/or professionals misunderstanding and/or simply missing what they hear. The professionals also cannot easily identify patterns in the individual to accurately and efficiently identify the individual's needs. And often the advice that the professionals give to the individual only marginally help the individual without addressing the underlying need.

In the present disclosure, systems and methods are provided for accurately and efficiently identifying insights about the individual to improve their well-being. The systems and methods can provide general wellness recommendations for the individual that address the needs of the individual and encourage the individual to live a healthy lifestyle. By utilizing machine learning models that are tailored to identify habit recognition and generate insights about the individual and provide recommendations, the systems and methods can remove barriers that exist in the existing systems and methods. When needed, the individual can also be connected with various professionals that can provide additional help to the individual.

Well-being is also a characteristic of groups or populations and can be affected by factors, shared by, in common to, or similar among, the members of the group or population. Such groups, affected by similar factors may include, by way of examples, members of an organization (such as a team, company, school, college, or university), individuals involved in a conflict, individuals involved in a natural disaster, and individuals in a geographic region. The well-being of groups or populations, and the factors in common that the affect well-being of such groups, also change and evolve over time.

In one aspect, disclosed herein are computer-implemented methods comprising: receiving media comprising an unstructured user-generated narrative; applying one or more algorithms to the unstructured user-generated narrative to extract semi-structured user context data; applying a first machine learning model to classify one or more of sentiment, intent, habits, patterns, beliefs, and motivations from at least the user context data, wherein the first machine learning model comprises an unsupervised machine learning model; applying a second machine learning model to identify one or more recommended next actions from at least the user context data, wherein the second machine learning model comprises a supervised machine learning model; and generating one or more well-being-related insights for the user based at least on the user context data, and one or more of the sentiment, intent, habits, patterns, beliefs, motivations, and one or more recommended next actions. In some embodiments, the media comprises audio. In some embodiments, the media comprises video. In some embodiments, the method further comprises providing one or more prompts to guide the user in creating the user-generated narrative. In further embodiments, the one or more prompts are generated by a machine learning model. In some embodiments, the method further comprises applying one or more quality metrics to the media. In some embodiments, the method further comprises transcoding the user-generated narrative. In some embodiments, the method further comprises transcribing the user-generated narrative. In various embodiments, the user context data comprises one or more of: a personality metric, a personal theme, a speech pattern, a stress metric, a user motivation, and an emotional well-being metric. In some embodiments, the method further comprises applying a third machine learning model to generate media comprising a summary of the unstructured user-generated narrative. In some embodiments, the one or more well-being-related insights comprise a prediction. In some embodiments, the one or more well-being-related insights comprise detection of suicidal ideation. In some embodiments, the user context data comprise sensor data. In further embodiments, the sensor comprises a wearable sensor and the sensor data comprise wearable sensor data. In various further embodiments, the wearable sensor data comprise one or more of: heart rate, heart rate variability, activity data, and sleep data. In some embodiments, the method further comprises providing general wellness curricula for the user based on the one or more well-being-related insights. In further embodiments, the method further comprises calculating a well-being index score based at least on the wearable sensor data and one or more user interactions with the general wellness curricula. In some embodiments, generating the one or more well-being-related insights for the user is further based on one or more user-generated reviews. In some embodiments, generating the one or more well-being-related insights for the user is further based on one or more user question and answer sessions. In some embodiments, generating the one or more well-being-related insights for the user is further based on one or more user-generated check-ins. In some embodiments, the method further comprises awarding tokens to the user for providing a user-generated narrative and providing a marketplace wherein the tokens are redeemable for items. In further embodiments, the items comprise non-fungible tokens (NFTs). In some embodiments, the method further comprises providing a healthcare provider portal comprising the one or more well-being-related insights for a healthcare provider associated with the user.

In another aspect, disclosed herein are computer-implemented systems comprising a computing device comprising at least one processor and instructions executable to cause the at least one processor to perform operations comprising: receiving media comprising an unstructured user-generated narrative; applying one or more algorithms to the unstructured user-generated narrative to extract semi-structured user context data; applying a first machine learning model to classify one or more of sentiment, intent, patterns, beliefs, and motivations from at least the user context data, wherein the first machine learning model comprises an unsupervised machine learning model; applying a second machine learning model to identify one or more recommended next actions from at least the user context data, wherein the second machine learning model comprises a supervised machine learning model; and generating one or more well-being-related insights for the user based at least on the user context data, and one or more of the sentiment, intent, patterns, beliefs, motivations, and one or more recommended next actions. In some embodiments, the media comprises audio. In some embodiments, the media comprises video. In some embodiments, the operations further comprise providing one or more prompts to guide the user in creating the user-generated narrative. In further embodiments, the one or more prompts are generated by a machine learning model. In some embodiments, the operations further comprise applying one or more quality metrics to the media. In some embodiments, the operations further comprise transcoding the user-generated narrative. In some embodiments, the operations further comprise transcribing the user-generated narrative. In various embodiments, the user context data comprises one or more of: a personality metric, a personal theme, a speech pattern, a stress metric, a user motivation, and an emotional well-being metric. In some embodiments, the operations further comprise applying a third machine learning model to generate media comprising a summary of the unstructured user-generated narrative. In some embodiments, the one or more well-being-related insights comprise a prediction. In some embodiments, the one or more well-being-related insights comprise detection of suicidal ideation. In some embodiments, the user context data comprise sensor data. In further embodiments, the sensor comprises a wearable sensor and the sensor data comprise wearable sensor data. In various further embodiments, the wearable sensor data comprise one or more of: heart rate, heart rate variability, activity data, and sleep data. In some embodiments, the operations further comprise providing general wellness curricula for the user based on the one or more well-being-related insights. In further embodiments, the operations further comprise calculating a well-being index score based at least on the wearable sensor data and one or more user interactions with the general wellness curricula. In some embodiments, generating the one or more well-being-related insights for the user is further based on one or more user-generated reviews. In some embodiments, generating the one or more well-being-related insights for the user is further based on one or more user question and answer sessions. In some embodiments, generating the one or more well-being-related insights for the user is further based on one or more user-generated check-ins. In some embodiments, the operations further comprise awarding tokens to the user for providing a user-generated narrative and providing a marketplace wherein the tokens are redeemable for items. In further embodiments, the items comprise non-fungible tokens (NFTs). In some embodiments, the operations further comprise providing a healthcare provider portal comprising the one or more well-being-related insights for a healthcare provider associated with the user.

In another aspect, disclosed herein are non-transitory computer-readable storage media encoded with instructions executable by at least one processor to provide a well-being application comprising: a recording studio module configured to receive media comprising an unstructured user-generated narrative; a user context extraction module configured to apply one or more algorithms to the unstructured user-generated narrative to extract semi-structured user context data; a wisdom engine module configured to: apply a first machine learning model to classify one or more of sentiment, intent, patterns, beliefs, and motivations from at least the user context data, wherein the first machine learning model comprises an unsupervised machine learning model; and apply a second machine learning model to identify one or more recommended next actions from at least the user context data, wherein the second machine learning model comprises a supervised machine learning model; and an insight generation module configured to generate one or more well-being-related insights for the user based at least on the user context data, and one or more of the sentiment, intent, patterns, beliefs, motivations, and one or more recommended next actions. In some embodiments, the media comprises audio. In some embodiments, the media comprises video. In some embodiments, the recording studio module is further configured to provide one or more prompts to guide the user in creating the user-generated narrative. In further embodiments, the one or more prompts are generated by a machine learning model. In some embodiments, the recording studio module or the user context extraction module is further configured to apply one or more quality metrics to the media. In some embodiments, the user context extraction module is further configured to transcode the user-generated narrative. In some embodiments, the user context extraction module is further configured to transcribe the user-generated narrative. In various embodiments, the user context data comprises one or more of: a personality metric, a personal theme, a speech pattern, a stress metric, a user motivation, and an emotional well-being metric. In some embodiments, the application further comprises a summary module configured to apply a third machine learning model to generate media comprising a summary of the unstructured user-generated narrative. In some embodiments, the one or more well-being-related insights comprise a prediction. In some embodiments, the one or more well-being-related insights comprise detection of suicidal ideation. In some embodiments, the user context data comprise sensor data. In further embodiments, the sensor comprises a wearable sensor and the sensor data comprise wearable sensor data. In various further embodiments, the wearable sensor data comprise one or more of: heart rate, heart rate variability, activity data, and sleep data. In some embodiments, the application further comprises a curriculum module configured to provide general wellness curricula for the user based on the one or more well-being-related insights. In further embodiments, the curriculum module is further configured to calculate a well-being index score based at least on the wearable sensor data and one or more user interactions with the general wellness curricula. In some embodiments, generating the one or more well-being-related insights for the user is further based on one or more user-generated reviews. In some embodiments, generating the one or more well-being-related insights for the user is further based on one or more user question and answer sessions. In some embodiments, generating the one or more well-being-related insights for the user is further based on one or more user-generated check-ins. In some embodiments, the application further comprises an incentive module configured to: award tokens to the user for providing a user-generated narrative; and provide a marketplace wherein the tokens are redeemable for items. In further embodiments, the items comprise non-fungible tokens (NFTs). In some embodiments, the application further comprises a healthcare provider portal comprising the one or more well-being-related insights for a healthcare provider associated with the user.

### BRIEF DESCRIPTION OF THE DRAWINGS

A better understanding of the features and advantages of the present subject matter will be obtained by reference to the following detailed description that sets forth illustrative embodiments and the accompanying drawings of which:

FIG. 1 shows a non-limiting example of a computing device; in this case, a device with one or more processors, memory, storage, and a network interface;

FIG. 2 shows a non-limiting example of a web/mobile application provision system; in this case, a system providing browser-based and/or native mobile user interfaces;

FIG. 3 shows a non-limiting example of a cloud-based web/mobile application provision system; in this case, a system comprising an elastically load balanced, auto-scaling web server and application server resources as well synchronously replicated databases;

FIG. 4A shows a schematic overview of an exemplary system providing a well-being platform comprising associated components and applications;

FIG. 4B shows a schematic overview of an exemplary architecture for providing a well-being platform comprising associated components and applications;

FIG. 5 shows a non-limiting example of a graphical user interface (GUI) for a recording studio module allowing a user to record an unstructured user-generated narrative; in this case, a GUI displaying a first introduction page of a first topic of what a story is;

FIG. 6 shows a non-limiting example of a GUI for a recording studio module allowing a user to record an unstructured user-generated narrative; in this case, a GUI displaying a prompt page for the first topic;

FIG. 7 shows a non-limiting example of a GUI for a recording studio module allowing a user to record an unstructured user-generated narrative; in this case, a GUI displaying a recording page for the first topic;

FIG. 8 shows a non-limiting example of a GUI for a recording studio module allowing a user to record an unstructured user-generated narrative; in this case, a GUI displaying a second introduction page of a second topic of describing the user;

FIG. 9 shows a non-limiting example of a GUI for a recording studio module allowing a user to record an unstructured user-generated narrative; in this case, a GUI displaying a prompt page of the second topic;

FIG. 10 shows a non-limiting example of a GUI for a recording studio module allowing a user to record an unstructured user-generated narrative; in this case, a GUI displaying a recording page of the third topic;

FIG. 11 shows a non-limiting example of a GUI for a recording studio module allowing a user to record an unstructured user-generated narrative; in this case, a GUI displaying a third introduction page of the third topic of describing the user's family;

FIG. 12 shows a non-limiting example of a GUI for a recording studio module allowing a user to record an unstructured user-generated narrative; in this case, a GUI displaying a prompt page of the third topic;

FIG. 13 shows a non-limiting example of a GUI for a recording studio module allowing a user to record an unstructured user-generated narrative; in this case, a GUI displaying a recording page of the third topic;

FIG. 14 shows a non-limiting example of a GUI for a recording studio module allowing a user to record an unstructured user-generated narrative; in this case, a GUI displaying a fourth introduction page of a fourth topic of describing the expectations;

FIG. 15 shows a non-limiting example of a GUI for a recording studio module allowing a user to record an unstructured user-generated narrative; in this case, a GUI displaying a prompt page of the fourth topic;

FIG. 16 shows a non-limiting example of a GUI for a recording studio module allowing a user to record an unstructured user-generated narrative; in this case, a GUI displaying a recording page of the fourth topic;

FIG. 17 shows a non-limiting example of a GUI for a recording studio module allowing a user to record an unstructured user-generated narrative; in this case, a GUI displaying a fifth introduction page of a fifth topic of describing the user's feelings;

FIG. 18 shows a non-limiting example of a GUI for a recording studio module allowing a user to record an unstructured user-generated narrative; in this case, a GUI displaying a prompt page of the fourth topic;

FIG. 19 shows a non-limiting example of a GUI for a recording studio module allowing a user to record an unstructured user-generated narrative; in this case, a GUI displaying a recording page of the fifth topic;

FIG. 20 shows a non-limiting example of a GUI for a well-being application; in this case, a GUI displaying a splash screen;

FIG. 21 shows a non-limiting example of a GUI for a well-being application; in this case, a GUI displaying an account creation screen;

FIG. 22 shows a non-limiting example of a GUI for a well-being application; in this case, a GUI displaying a data usage information screen providing access to a privacy policy;

FIG. 23 shows a non-limiting example of a GUI for a well-being application; in this case, a GUI displaying a privacy and security consent screen providing access to terms and conditions and a privacy policy;

FIGS. 24-26 show a non-limiting example of a GUI for a well-being application; in this case, a GUI displaying a home screen;

FIG. 27 shows a non-limiting example of a GUI for a well-being application; in this case, a GUI displaying an enhanced human insights (EHI) screen;

FIG. 28 shows a non-limiting example of a GUI for a well-being application; in this case, a GUI displaying an audio and video recording screen;

FIG. 29 shows a non-limiting example of a GUI for a well-being application; in this case, a GUI displaying an exploration screen allowing a user to browse page content;

FIG. 30 shows a non-limiting example of a GUI for a well-being application; in this case, a GUI displaying a profile screen providing access to recorded moments and reviews;

FIG. 31 shows a non-limiting example of a GUI for a well-being application; in this case, a GUI displaying a settings screen;

FIGS. 32-39C show various views of the interior and exterior of the Wisdom Pod;

FIG. 40 shows a non-limiting example of a schematic diagram; in this case, a schematic diagram illustrating an overview of an AI-driven artifact generation service described herein;

FIG. 41 shows a non-limiting example of an architecture and process flow diagram; in this case, an architecture and process flow diagram illustrating an AI-driven artifact generation service described herein;

FIGS. 42-43 show non-limiting examples of still image artwork generated, based on individual and/or group stories shared, by the generative AI technology described herein;

FIGS. 44A-44G show a first non-limiting example of video artwork generated (as captured as series of still images), based on individual and/or group stories shared, by the generative AI technology described herein;

FIGS. 45A-45G show a second non-limiting example of video artwork generated (as captured as series of still images), based on individual and/or group stories shared, by the generative AI technology described herein;

FIGS. 46A-46I show a non-limiting example of insights that are derived from the stories provided by an individual;

FIGS. 47A-47I show a non-limiting example of insights that are derived from the stories provided by a group of individuals; and

FIGS. 48A-48D show a non-limiting example of a general workflow for implementing the described technology.

### DETAILED DESCRIPTION

Described herein, in certain embodiments, are computer-implemented methods comprising: receiving media comprising an unstructured user-generated narrative; applying one or more algorithms to the unstructured user-generated narrative to extract semi-structured user context data; applying a first machine learning model to classify one or more of sentiment, intent, patterns, beliefs, and motivations from at least the user context data, wherein the first machine learning model comprises an unsupervised machine learning model; applying a second machine learning model to identify one or more recommended next actions from at least the user context data, wherein the second machine learning model comprises a supervised machine learning model; and generating one or more well-being-related insights for the user based at least on the user context data, and one or more of the sentiment, intent, patterns, beliefs, motivations, and one or more recommended next actions.

Also described herein, in certain embodiments, are computer-implemented systems comprising a computing device comprising at least one processor and instructions executable to cause the at least one processor to perform operations comprising: receiving media comprising an unstructured user-generated narrative; applying one or more algorithms to the unstructured user-generated narrative to extract semi-structured user context data; applying a first machine learning model to classify one or more of sentiment, intent, patterns, beliefs, and motivations from at least the user context data, wherein the first machine learning model comprises an unsupervised machine learning model; applying a second machine learning model to identify one or more recommended next actions from at least the user context data, wherein the second machine learning model comprises a supervised machine learning model; and generating one or more well-being-related insights for the user based at least on the user context data, and one or more of the sentiment, intent, patterns, beliefs, motivations, and one or more recommended next actions.

Also described herein, in certain embodiments, are non-transitory computer-readable storage media encoded with instructions executable by at least one processor to provide a well-being application comprising: a recording studio module configured to receive media comprising an unstructured user-generated narrative; a user context extraction module configured to apply one or more algorithms to the unstructured user-generated narrative to extract semi-structured user context data; a wisdom engine module configured to: apply a first machine learning model to classify one or more of sentiment, intent, patterns, beliefs, and motivations from at least the user context data, wherein the first machine learning model comprises an unsupervised machine learning model; and apply a second machine learning model to identify one or more recommended next actions from at least the user context data, wherein the second machine learning model comprises a supervised machine learning model; and an insight generation module configured to generate one or more well-being-related insights for the user based at least on the user context data, and one or more of the sentiment, intent, patterns, beliefs, motivations, and one or more recommended next actions.

### Certain Definitions

Unless otherwise defined, all technical terms used herein have the same meaning as commonly understood by one of ordinary skill in the art to which the present subject matter belongs.

As used in this specification and the appended claims, the singular forms “a,” “an,” and “the” include plural references unless the context clearly dictates otherwise. Any reference to “or” herein is intended to encompass “and/or” unless otherwise stated.

Reference throughout this specification to “some embodiments,” “further embodiments,” or “a particular embodiment,” means that a particular feature, structure, or characteristic described in connection with the embodiment is included in at least one embodiment. Thus, the appearances of the phrase “in some embodiments,” or “in further embodiments,” or “in a particular embodiment” in various places throughout this specification are not necessarily all referring to the same embodiment. Furthermore, the particular features, structures, or characteristics may be combined in any suitable manner in one or more embodiments.

### Computing System

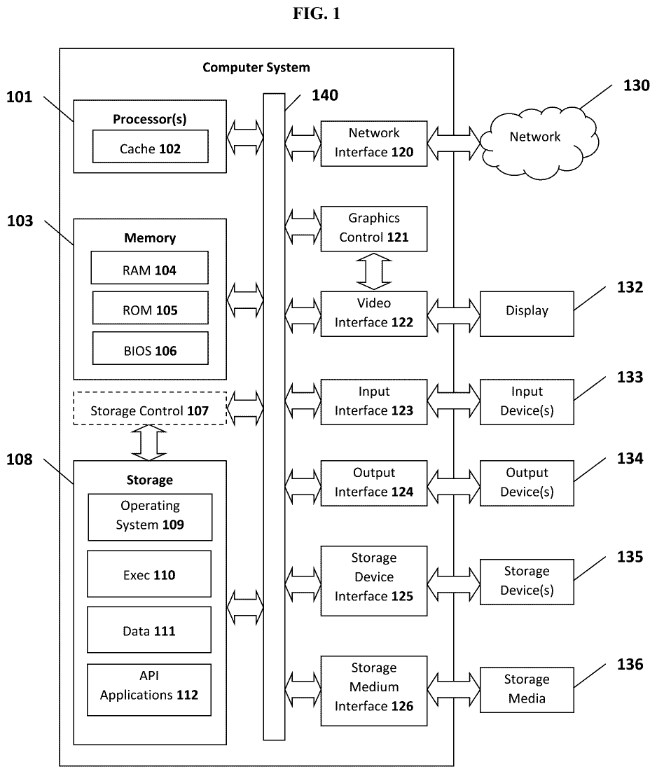

Referring to FIG. 1, a block diagram is shown depicting an exemplary machine that includes a computer system 100 (e.g., a processing or computing system) within which a set of instructions can execute for causing a device to perform or execute any one or more of the aspects and/or methodologies for static code scheduling of the present disclosure. The components in FIG. 1 are examples only and do not limit the scope of use or functionality of any hardware, software, embedded logic component, or a combination of two or more such components implementing particular embodiments.

Computer system 100 may include one or more processors 101, a memory 103, and a storage 108 that communicate with each other, and with other components, via a bus 140. The bus 140 may also link a display 132, one or more input devices 133 (which may, for example, include a keypad, a keyboard, a mouse, a stylus, etc.), one or more output devices 134, one or more storage devices 135, and various tangible storage media 136. All of these elements may interface directly or via one or more interfaces or adaptors to the bus 140. For instance, the various tangible storage media 136 can interface with the bus 140 via storage medium interface 126. Computer system 100 may have any suitable physical form, including but not limited to one or more integrated circuits (ICs), printed circuit boards (PCBs), mobile handheld devices (such as mobile telephones or PDAs), laptop or notebook computers, distributed computer systems, computing grids, or servers.

Computer system 100 includes one or more processor(s) 101 (e.g., central processing units (CPUs), general purpose graphics processing units (GPGPUs), or quantum processing units (QPUs)) that carry out functions. Processor(s) 101 optionally contains a cache memory unit 102 for temporary local storage of instructions, data, or computer addresses. Processor(s) 101 are configured to assist in execution of computer readable instructions. Computer system 100 may provide functionality for the components depicted in FIG. 1 as a result of the processor(s) 101 executing non-transitory, processor-executable instructions embodied in one or more tangible computer-readable storage media, such as memory 103, storage 108, storage devices 135, and/or storage medium 136. The computer-readable media may store software that implements particular embodiments, and processor(s) 101 may execute the software. Memory 103 may read the software from one or more other computer-readable media (such as mass storage device(s) 135, 136) or from one or more other sources through a suitable interface, such as network interface 120. The software may cause processor(s) 101 to carry out one or more processes or one or more steps of one or more processes described or illustrated herein. Carrying out such processes or steps may include defining data structures stored in memory 103 and modifying the data structures as directed by the software.

The memory 103 may include various components (e.g., machine readable media) including, but not limited to, a random access memory component (e.g., RAM 104) (e.g., static RAM (SRAM), dynamic RAM (DRAM), ferroelectric random access memory (FRAM), phase-change random access memory (PRAM), etc.), a read-only memory component (e.g., ROM 105), and any combinations thereof. ROM 105 may act to communicate data and instructions unidirectionally to processor(s) 101, and RAM 104 may act to communicate data and instructions bidirectionally with processor(s) 101. ROM 105 and RAM 104 may include any suitable tangible computer-readable media described below. In one example, a basic input/output system 106 (BIOS), including basic routines that help to transfer information between elements within computer system 100, such as during start-up, may be stored in the memory 103.

Fixed storage 108 is connected bidirectionally to processor(s) 101, optionally through storage control unit 107. Fixed storage 108 provides additional data storage capacity and may also include any suitable tangible computer-readable media described herein. Storage 108 may be used to store operating system 109, executable(s) 110, data 111, applications 112 (application programs), and the like. Storage 108 can also include an optical disk drive, a solid-state memory device (e.g., flash-based systems), or a combination of any of the above. Information in storage 108 may, in appropriate cases, be incorporated as virtual memory in memory 103.

In one example, storage device(s) 135 may be removably interfaced with computer system 100 (e.g., via an external port connector (not shown)) via a storage device interface 125. Particularly, storage device(s) 135 and an associated machine-readable medium may provide non-volatile and/or volatile storage of machine-readable instructions, data structures, program modules, and/or other data for the computer system 100. In one example, software may reside, completely or partially, within a machine-readable medium on storage device(s) 135. In another example, software may reside, completely or partially, within processor(s) 101.

Bus 140 connects a wide variety of subsystems. Herein, reference to a bus may encompass one or more digital signal lines serving a common function, where appropriate. Bus 140 may be any of several types of bus structures including, but not limited to, a memory bus, a memory controller, a peripheral bus, a local bus, and any combinations thereof, using any of a variety of bus architectures. As an example and not by way of limitation, such architectures include an Industry Standard Architecture (ISA) bus, an Enhanced ISA (EISA) bus, a Micro Channel Architecture (MCA) bus, a Video Electronics Standards Association local bus (VLB), a Peripheral Component Interconnect (PCI) bus, a PCI-Express (PCI-X) bus, an Accelerated Graphics Port (AGP) bus, HyperTransport (HTX) bus, serial advanced technology attachment (SATA) bus, and any combinations thereof.

Computer system 100 may also include an input device 133. In one example, a user of computer system 100 may enter commands and/or other information into computer system 100 via input device(s) 133. Examples of an input device(s) 133 include, but are not limited to, an alpha-numeric input device (e.g., a keyboard), a pointing device (e.g., a mouse or touchpad), a touchpad, a touch screen, a multi-touch screen, a joystick, a stylus, a gamepad, an audio input device (e.g., a microphone, a voice response system, etc.), an optical scanner, a video or still image capture device (e.g., a camera), and any combinations thereof. In some embodiments, the input device is a Kinect, Leap Motion, or the like. Input device(s) 133 may be interfaced to bus 140 via any of a variety of input interfaces 123 (e.g., input interface 123) including, but not limited to, serial, parallel, game port, USB, FIREWIRE, THUNDERBOLT, or any combination of the above.

In particular embodiments, when computer system 100 is connected to network 130, computer system 100 may communicate with other devices, specifically mobile devices and enterprise systems, distributed computing systems, cloud storage systems, cloud computing systems, and the like, connected to network 130. Communications to and from computer system 100 may be sent through network interface 120. For example, network interface 120 may receive incoming communications (such as requests or responses from other devices) in the form of one or more packets (such as Internet Protocol (IP) packets) from network 130, and computer system 100 may store the incoming communications in memory 103 for processing. Computer system 100 may similarly store outgoing communications (such as requests or responses to other devices) in the form of one or more packets in memory 103 and communicated to network 130 from network interface 120. Processor(s) 101 may access these communication packets stored in memory 103 for processing.

Examples of the network interface 120 include, but are not limited to, a network interface card, a modem, and any combination thereof. Examples of a network 130 or network segment 130 include, but are not limited to, a distributed computing system, a cloud computing system, a wide area network (WAN) (e.g., the Internet, an enterprise network), a local area network (LAN) (e.g., a network associated with an office, a building, a campus or other relatively small geographic space), a telephone network, a direct connection between two computing devices, a peer-to-peer network, and any combinations thereof. A network, such as network 130, may employ a wired and/or a wireless mode of communication. In general, any network topology may be used.

Information and data can be displayed through a display 132. Examples of a display 132 include, but are not limited to, a cathode ray tube (CRT), a liquid crystal display (LCD), a thin film transistor liquid crystal display (TFT-LCD), an organic liquid crystal display (OLED) such as a passive-matrix OLED (PMOLED) or active-matrix OLED (AMOLED) display, a plasma display, and any combinations thereof. The display 132 can interface to the processor(s) 101, memory 103, and fixed storage 108, as well as other devices, such as input device(s) 133, via the bus 140. The display 132 is linked to the bus 140 via a video interface 122, and transport of data between the display 132 and the bus 140 can be controlled via the graphics control 121. In some embodiments, the display is a video projector. In some embodiments, the display is a head-mounted display (HMD) such as a VR headset. In further embodiments, suitable VR headsets include, by way of non-limiting examples, HTC Vive, Oculus Rift, Samsung Gear VR, Microsoft HoloLens, Razer OSVR, FOVE VR, Zeiss VR One, Avegant Glyph, Freefly VR headset, and the like. In still further embodiments, the display is a combination of devices such as those disclosed herein.

In addition to a display 132, computer system 100 may include one or more other peripheral output devices 134 including, but not limited to, an audio speaker, a printer, a storage device, and any combinations thereof. Such peripheral output devices may be connected to the bus 140 via an output interface 124. Examples of an output interface 124 include, but are not limited to, a serial port, a parallel connection, a USB port, a FIREWIRE port, a THUNDERBOLT port, and any combinations thereof.

In addition or as an alternative, computer system 100 may provide functionality as a result of logic hardwired or otherwise embodied in a circuit, which may operate in place of or together with software to execute one or more processes or one or more steps of one or more processes described or illustrated herein. Reference to software in this disclosure may encompass logic, and reference to logic may encompass software. Moreover, reference to a computer-readable medium may encompass a circuit (such as an IC) storing software for execution, a circuit embodying logic for execution, or both, where appropriate. The present disclosure encompasses any suitable combination of hardware, software, or both.

Those of skill in the art will appreciate that the various illustrative logical blocks, modules, circuits, and algorithm steps described in connection with the embodiments disclosed herein may be implemented as electronic hardware, computer software, or combinations of both. To clearly illustrate this interchangeability of hardware and software, various illustrative components, blocks, modules, circuits, and steps have been described above generally in terms of their functionality.

The various illustrative logical blocks, modules, and circuits described in connection with the embodiments disclosed herein may be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor may be a microprocessor, but in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

The steps of a method or algorithm described in connection with the embodiments disclosed herein may be embodied directly in hardware, in a software module executed by one or more processor(s), or in a combination of the two. A software module may reside in RAM memory, flash memory, ROM memory, EPROM memory, EEPROM memory, registers, hard disk, a removable disk, a CD-ROM, or any other form of storage medium known in the art. An exemplary storage medium is coupled to the processor such the processor can read information from, and write information to, the storage medium. In the alternative, the storage medium may be integral to the processor. The processor and the storage medium may reside in an ASIC. The ASIC may reside in a user terminal. In the alternative, the processor and the storage medium may reside as discrete components in a user terminal.

In accordance with the description herein, suitable computing devices include, by way of non-limiting examples, server computers, desktop computers, laptop computers, notebook computers, sub-notebook computers, netbook computers, netpad computers, set-top computers, media streaming devices, handheld computers, Internet appliances, mobile smartphones, tablet computers, personal digital assistants, video game consoles, and vehicles. Those of skill in the art will also recognize that select televisions, video players, and digital music players with optional computer network connectivity are suitable for use in the system described herein. Suitable tablet computers, in various embodiments, include those with booklet, slate, and convertible configurations, known to those of skill in the art.

In some embodiments, the computing device includes an operating system configured to perform executable instructions. The operating system is, for example, software, including programs and data, which manages the device's hardware and provides services for execution of applications. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft® Windows®, Apple® Mac OS X®, UNIX®, and UNIX-like operating systems such as GNU/Linux®. In some embodiments, the operating system is provided by cloud computing. Those of skill in the art will also recognize that suitable mobile smartphone operating systems include, by way of non-limiting examples, Nokia® Symbian® OS, Apple® iOS®, Research In Motion® BlackBerry OS®, Google® Android®, Microsoft® Windows Phone® OS, Microsoft® Windows Mobile® OS, Linux®, and Palm® WebOS®. Those of skill in the art will also recognize that suitable media streaming device operating systems include, by way of non-limiting examples, Apple TV®, Roku®, Boxee®, Google TV®, Google Chromecast®, Amazon Fire®, and Samsung® HomeSync®. Those of skill in the art will also recognize that suitable video game console operating systems include, by way of non-limiting examples, Sony® PS3®, Sony® PS4®, Microsoft® Xbox 360®, Microsoft Xbox One, Nintendo® Wii®, Nintendo® Wii U®, and Ouya®.

### Non-Transitory Computer Readable Storage Medium (CRM)

In some embodiments, the platforms, systems, media, and methods disclosed herein include one or more non-transitory computer readable storage media encoded with a program including instructions executable by the operating system of an optionally networked computing device. In further embodiments, a computer readable storage medium is a tangible component of a computing device. In still further embodiments, a computer readable storage medium is optionally removable from a computing device. In some embodiments, a computer readable storage medium includes, by way of non-limiting examples, CD-ROMs, DVDs, flash memory devices, solid state memory, magnetic disk drives, magnetic tape drives, optical disk drives, distributed computing systems including cloud computing systems and services, and the like. In some cases, the program and instructions are permanently, substantially permanently, semi-permanently, or non-transitorily encoded on the media.

### Computer Program

In some embodiments, the platforms, systems, media, and methods disclosed herein include at least one computer program, or use of the same. A computer program includes a sequence of instructions, executable by one or more processor(s) of the computing device's CPU, written to perform a specified task. Computer readable instructions may be implemented as program modules, such as functions, objects, Application Programming Interfaces (APIs), computing data structures, and the like, which perform particular tasks or implement particular abstract data types. In light of the disclosure provided herein, those of skill in the art will recognize that a computer program may be written in various versions of various languages.

The functionality of the computer readable instructions may be combined or distributed as desired in various environments. In some embodiments, a computer program comprises one sequence of instructions. In some embodiments, a computer program comprises a plurality of sequences of instructions. In some embodiments, a computer program is provided from one location. In other embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof.

### Web Application

In some embodiments, a computer program includes a web application. In light of the disclosure provided herein, those of skill in the art will recognize that a web application, in various embodiments, utilizes one or more software frameworks and one or more database systems. In some embodiments, a web application is created upon a software framework such as Microsoft®.NET or Ruby on Rails (RoR). In some embodiments, a web application utilizes one or more database systems including, by way of non-limiting examples, relational, non-relational, object oriented, associative, XML, and document oriented database systems. In further embodiments, suitable relational database systems include, by way of non-limiting examples, Microsoft® SQL Server, mySQL™, and Oracle®. Those of skill in the art will also recognize that a web application, in various embodiments, is written in one or more versions of one or more languages. A web application may be written in one or more markup languages, presentation definition languages, client-side scripting languages, server-side coding languages, database query languages, or combinations thereof. In some embodiments, a web application is written to some extent in a markup language such as Hypertext Markup Language (HTML), Extensible Hypertext Markup Language (XHTML), or extensible Markup Language (XML). In some embodiments, a web application is written to some extent in a presentation definition language such as Cascading Style Sheets (CSS). In some embodiments, a web application is written to some extent in a client-side scripting language such as Asynchronous JavaScript and XML (AJAX), Flash® ActionScript, JavaScript, or Silverlight®. In some embodiments, a web application is written to some extent in a server-side coding language such as Active Server Pages (ASP), ColdFusion®, Perl, Java™, JavaServer Pages (JSP), Hypertext Preprocessor (PHP), Python™, Ruby, Tcl, Smalltalk, WebDNA®, or Groovy. In some embodiments, a web application is written to some extent in a database query language such as Structured Query Language (SQL). In some embodiments, a web application integrates enterprise server products such as IBM® Lotus Domino®. In some embodiments, a web application includes a media player element. In various further embodiments, a media player element utilizes one or more of many suitable multimedia technologies including, by way of non-limiting examples, Adobe® Flash®, HTML 5, Apple® QuickTime®, Microsoft® Silverlight®, Java™, and Unity®

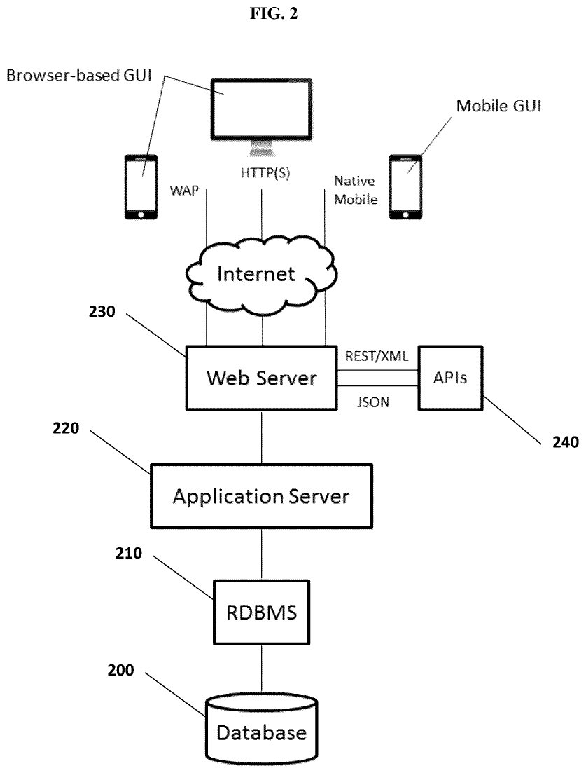

Referring to FIG. 2, in a particular embodiment, an application provision system comprises one or more databases 200 accessed by a relational database management system (RDBMS) 210. Suitable RDBMSs include Firebird, MySQL, PostgreSQL, SQLite, Oracle Database, Microsoft SQL Server, IBM DB2, IBM Informix, SAP Sybase, Teradata, and the like. In this embodiment, the application provision system further comprises one or more application severs 220 (such as Java servers, .NET servers, PHP servers, and the like) and one or more web servers 230 (such as Apache, IIS, GWS and the like). The web server(s) optionally expose one or more web services via app application programming interfaces (APIs) 240. Via a network, such as the Internet, the system provides browser-based and/or mobile native user interfaces.

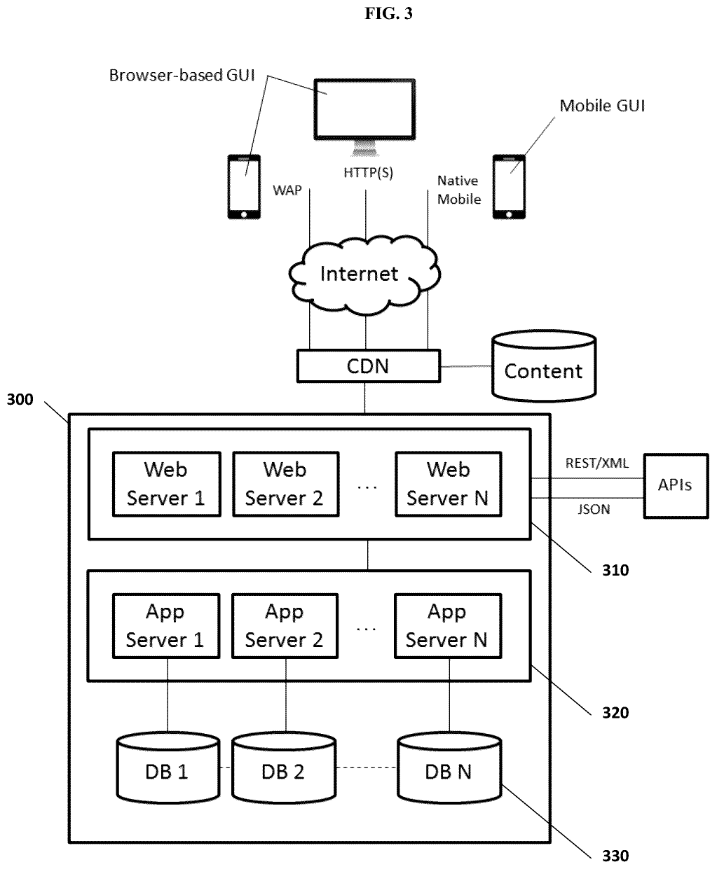

Referring to FIG. 3, in a particular embodiment, an application provision system alternatively has a distributed, cloud-based architecture 300 and comprises elastically load balanced, auto-scaling web server resources 310 and application server resources 320 as well synchronously replicated databases 330.

### Mobile Application

In some embodiments, a computer program includes a mobile application provided to a mobile computing device. In some embodiments, the mobile application is provided to a mobile computing device at the time it is manufactured. In other embodiments, the mobile application is provided to a mobile computing device via the computer network described herein.

In view of the disclosure provided herein, a mobile application is created by techniques known to those of skill in the art using hardware, languages, and development environments known to the art. Those of skill in the art will recognize that mobile applications are written in several languages. Suitable programming languages include, by way of non-limiting examples, C, C++, C#, Objective-C, Java™, JavaScript, Pascal, Object Pascal, Python™, Ruby, VB .NET, WML, and XHTML/HTML with or without CSS, or combinations thereof.

Suitable mobile application development environments are available from several sources. Commercially available development environments include, by way of non-limiting examples, AirplaySDK, alcheMo, Appcelerator®, Celsius, Bedrock, Flash Lite, .NET Compact Framework, Rhomobile, and WorkLight Mobile Platform. Other development environments are available without cost including, by way of non-limiting examples, Lazarus, MobiFlex, MoSync, and PhoneGap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of skill in the art will recognize that several commercial forums are available for distribution of mobile applications including, by way of non-limiting examples, Apple® App Store, Google® Play, Chrome WebStore, BlackBerry® App World, App Store for Palm devices, App Catalog for webOS, Windows® Marketplace for Mobile, Ovi Store for Nokia® devices, Samsung® Apps, and Nintendo® DSi Shop.

### Standalone Application

In some embodiments, a computer program includes a standalone application, which is a program that is run as an independent computer process, not an add-on to an existing process, e.g., not a plug-in. Those of skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB .NET, or combinations thereof. Compilation is often performed, at least in part, to create an executable program. In some embodiments, a computer program includes one or more executable complied applications.

### Web Browser Plug-In

In some embodiments, the computer program includes a web browser plug-in (e.g., extension, etc.). In computing, a plug-in is one or more software components that add specific functionality to a larger software application. Makers of software applications support plug-ins to enable third-party developers to create abilities which extend an application, to support easily adding new features, and to reduce the size of an application. When supported, plug-ins enable customizing the functionality of a software application. For example, plug-ins are commonly used in web browsers to play video, generate interactivity, scan for viruses, and display particular file types. Those of skill in the art will be familiar with several web browser plug-ins including, Adobe® Flash® Player, Microsoft® Silverlight®, and Apple® QuickTime®. In some embodiments, the toolbar comprises one or more web browser extensions, add-ins, or add-ons. In some embodiments, the toolbar comprises one or more explorer bars, tool bands, or desk bands.

In view of the disclosure provided herein, those of skill in the art will recognize that several plug-in frameworks are available that enable development of plug-ins in various programming languages, including, by way of non-limiting examples, C++, Delphi, Java™, PHP, Python™, and VB .NET, or combinations thereof.

Web browsers (also called Internet browsers) are software applications, designed for use with network-connected computing devices, for retrieving, presenting, and traversing information resources on the World Wide Web. Suitable web browsers include, by way of non-limiting examples, Microsoft® Internet Explorer®, Mozilla® Firefox®, Google® Chrome, Apple® Safari®, Opera Software® Opera®, and KDE Konqueror. In some embodiments, the web browser is a mobile web browser. Mobile web browsers (also called microbrowsers, mini-browsers, and wireless browsers) are designed for use on mobile computing devices including, by way of non-limiting examples, handheld computers, tablet computers, netbook computers, subnotebook computers, smartphones, music players, personal digital assistants (PDAs), and handheld video game systems. Suitable mobile web browsers include, by way of non-limiting examples, Google® Android® browser, RIM Blackberry® Browser, Apple® Safari®, Palm® Blazer, Palm® WebOS® Browser, Mozilla® Firefox® for mobile, Microsoft® Internet Explorer® Mobile, Amazon® Kindle® Basic Web, Nokia® Browser, Opera Software® Opera® Mobile, and Sony® PSP™ browser.

### Software Modules

In some embodiments, the platforms, systems, media, and methods disclosed herein include software, server, and/or database modules, or use of the same. In view of the disclosure provided herein, software modules are created by techniques known to those of skill in the art using machines, software, and languages known to the art. The software modules disclosed herein are implemented in a multitude of ways. In various embodiments, a software module comprises a file, a section of code, a programming object, a programming structure, a distributed computing resource, a cloud computing resource, or combinations thereof. In further various embodiments, a software module comprises a plurality of files, a plurality of sections of code, a plurality of programming objects, a plurality of programming structures, a plurality of distributed computing resources, a plurality of cloud computing resources, or combinations thereof. In various embodiments, the one or more software modules comprise, by way of non-limiting examples, a web application, a mobile application, a standalone application, and a distributed or cloud computing application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on a distributed computing platform such as a cloud computing platform. In some embodiments, software modules are hosted on one or more machines in one location. In other embodiments, software modules are hosted on one or more machines in more than one location.

### Databases

In some embodiments, the platforms, systems, media, and methods disclosed herein include one or more databases, or use of the same. In view of the disclosure provided herein, those of skill in the art will recognize that many databases are suitable for storage and retrieval of, for example, user, media, prompt, summary, curricula, review, survey, check-in, well-being index, token, and marketplace information (data and metadata). In some cases, the data comprises individual-level data, which may include time-series data for one or more individuals. In some cases, the data comprises group-level data and/or a population-level data, which may include time-series data for one or more groups and/or populations.

In various embodiments, suitable databases include, by way of non-limiting examples, relational databases, non-relational databases, object oriented databases, object databases, entity-relationship model databases, associative databases, XML databases, document oriented databases, and graph databases. Further non-limiting examples include SQL, PostgreSQL, MySQL, Oracle, DB2, Sybase, and MongoDB. In some embodiments, a database is Internet-based. In further embodiments, a database is web-based. In still further embodiments, a database is cloud computing-based. In a particular embodiment, a database is a distributed database. In other embodiments, a database is based on one or more local computer storage devices.

### Generating Insights and Recommended Next Actions

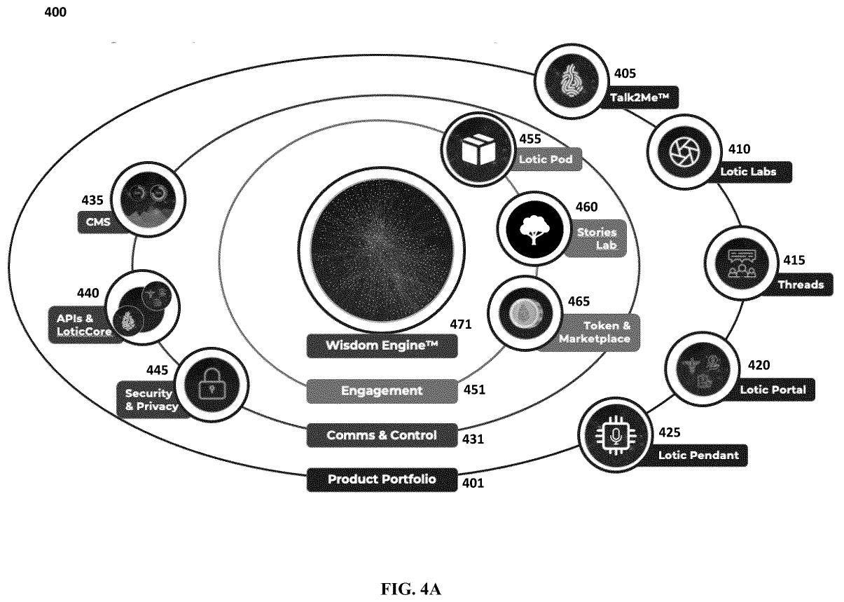

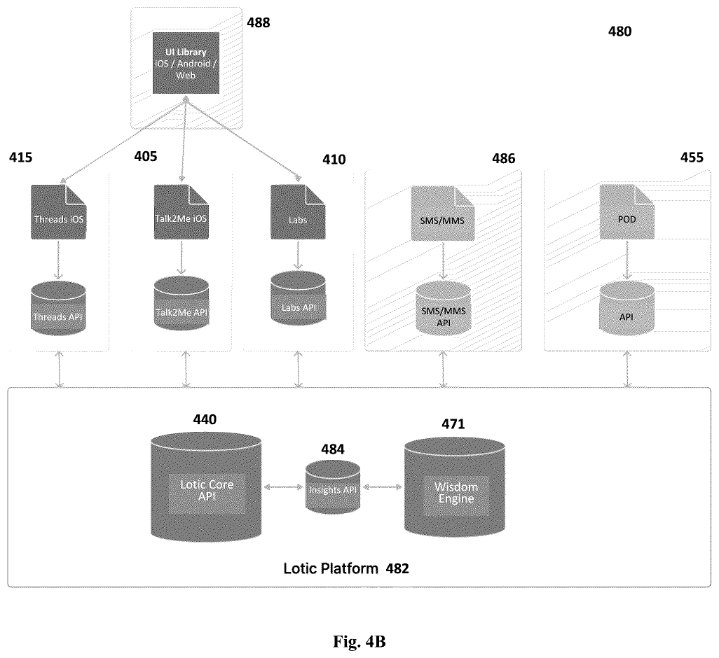

Referring to FIG. 4A, a block diagram of an exemplary system 400 is shown, in accordance with some embodiments. The system 400 of FIG. 4A includes one or more components that assist in discovering insights in users. The one or more components can include a user application (e.g., Talk2Me™ application) 405, Lotic Labs 410, Threads 415, provider portal (e.g., Lotic Portal™) 420, pairing devices (e.g., Lotic Pendant) 425, content management system (CMS) 435, application programming interfaces (APIs) 440, and security and privacy subsystem 445, product portfolio subsystem 401, communications & control subsystem 431, engagement subsystem 451, insight engine (e.g., Wisdom Engine™) 471, Lotic Pod (or Wisdom Pod) 455, stories workspace (e.g., Stories Lab™) 460, and engagement marketplace (e.g., Token & Marketplace) 465.

In some embodiments, the user application 405 can include a software application that can be installed on an electronic device such as a mobile device, computer, tablet, wearable device, smart glasses, etc. The user application 405 can include a recording tool that a user may access to record audio and/or video disclosures about a topic. The recording can be made in response to a prompt provided by the user application 405 or unprompted (e.g., free response, free form, etc.). In some embodiments, the recording can be saved locally to the device on which the user application 405 is running. In some embodiments, the recording can be streamed to a remote server that can save the recording. In some embodiments, the recording can have any length of time.

In some embodiments, the recording can be transmitted to a server that can analyze the recording. In some embodiments, the recording can be automatically transcribed into text. In some embodiments, the text can be analyzed by one or more machine learning models to provide one or more insights into the user.

In some embodiments, the provider portal 420 can provide the ability of healthcare professionals who interact with the users to access user data and information throughout all aspects of the care cycle. For example, the healthcare professionals can reliably track symptoms, monitor progress, and provide evidence-based healthcare and resources to the user.

In some embodiments, the pairing devices 425 can generally include custom hardware devices that can pin to the user's clothes. The pairing devices 425 can assist the user in recording the stories that the user shares.

In some embodiments, the product portfolio subsystem 401 can include one or more software modules that responsible for managing the user application 405, the Lotic Labs 410, Threads 415, the provider portal, and the pairing devices 425.

In some embodiments, the CMS 435 can include a variety of content that can help the users. For example, the CMS 435 can include curricula, classes, videos, etc. that can help treat and care for the user. The CMS 435 can provide recommendations for the user to take one or more curricula and one or more classes and/or watch one or more videos and/or read one or more articles based on the user's insights. Then based on the sensor data of the wearable data and/or whether the user has taken the classes and/or curricula and/or watched the videos and/or read the articles, the CMS 435 can generate a well-being index score of the user that can indicate the quality of the user's well-being.

In some embodiments, the APIs 440 can expose functionality of the present disclosure such that third-party developers and affiliates can utilize the information within the system 400.

In some embodiments, the security and privacy subsystem 445 can include encryption functionality that can encrypt the stories, user data, and other information that is used by the system 400. Furthermore, the security and privacy subsystem 445 can be responsible for managing user accounts and identities for the system 400.

In some embodiments, the communications & control subsystem 431 can include one or more software modules that are responsible for managing the CMS 435, the APIs 440, and security and privacy subsystem 445.

In some embodiments, the stories workspace 460 can include a workspace for various wellness professionals and others to share one or more ideas revolving around sharing stories. For example, a professional may share an article on how sharing stories can expedite the recovery of trauma. As another example, another professional may share an article on how storytelling can be a form of self-care and help with regulating one's emotions.

In some embodiments, the engagement marketplace 465 can include using tokens that are based on a distributed ledger technology (e.g., blockchain). In some embodiments, the users can use tokens to purchase products (e.g., drugs, books, etc.) and/or services (e.g., counseling sessions, etc.) from affiliate partners (e.g., vendors, pharmacies, etc.). In some embodiments, the engagement marketplace 465 can provide incentives to the users such that when the user purchases the products and/or services, the user is provided with rewards.

In some embodiments, the engagement subsystem 451 can include one or more software modules that are responsible for managing the Lotic Pod 455, the Stories Lab 460, and the engagement market place 465.

Referring to FIG. 4B, a block diagram of an architecture 480 is shown, in accordance with some embodiments. The exemplary architecture 480 of FIG. 4B includes the Lotic Platform 482 comprising the Lotic Insights API 484 in communication with both the Wisdom Engine 471 and the Lotic Core API 440. As shown in FIG. 4B, the Lotic Platform 482 supports a plurality of applications, including by way of non-limiting examples, the Lotic Pod 455, a SMS/MMS channel 486, as well as applications utilizing a front-end UI Library 488, such as Lotic Threads 415, Talk2Me 405, and Lotic Labs 410.

### Distributed Ledger Technology

A distributed ledger network (e.g., blockchain) may comprise a growing list of records, such as blocks, that may be linked and secured using cryptography. The database network system may be a distributed database system which may be collectively maintained by a plurality of nodes in a decentralized manner. The network may comprise a series of blocks which may be generated by, or with aid of, cryptography. The database network system may comprise or be an immutable digital public ledger. The database network system may be a continuously growing, distributed database. The distributed database may be cryptographically secured using the methods, procedures, and measures provided herein.

A blockchain may comprise one or more blocks. The one or more blocks may be associated with a sequence. Each block may contain a hash value of a previous block, a timestamp, and data (e.g., transaction data). The blockchain may be formed starting from a genesis block to a current block. The blocks may be generated in a chronological order, such that a hash value of the previous block may be known. In some cases, the blocks may be generated in a linear, chronological order. In some cases, blocks may be generated in a non-linear order or according to other patterns. The database network system may have substantially complete information from the genesis block to the most recently completed block.

In some cases, the database network system may store information comprising data in uniform-sized blocks. Each block may comprise hashed information from the previous block to provide cryptographic security. This may also be referred to as data hashing. Data hashing may comprise a hashing function. The hashed data and/or information may comprise the data and digital signatures, and/or keys (such as public key and/or private key) from the previous block, and the hashed information of the previous blocks that may go all the way back to the genesis block. The information may be distributed through a hash function which may then point to the address of the previous block. In some cases, the database network system may comprise a linked list that may comprise pointers. Blocks may store information validated by nodes that may be cryptographically secured according to the methods described herein.

The genesis block may be where the very first data in the network were generated. In some examples, the database network system may be a system of decentralized transactions and/or a system of decentralized trustless transactions. In some cases, the database network system may be a decentralized public ledger. For example, the database network system may stand as a trustless proof mechanism of all activities (such as transactions) on the network. Users may trust the system of the public ledger stored on many different decentralized nodes, in some cases worldwide, as opposed to establishing and maintaining trust with a counterparty, such as a transaction counterparty, such as another person, or a third-party intermediary (e.g., a bank).

The database network system may perform as another application layer to run on an existing stack of Internet protocols, adding a new tier to the Internet to enable activities such as performing economic transactions, currency payments such as digital currency payments, registering and maintaining financial contracts, transacting hard and/or soft assets, and more activities.

Further, the database network system may be used for activities beyond transactions, such as a registry and/or inventory system for recording, tracking, tracking, monitoring, and/or transacting of all assets. In some examples, the database network system may be like a ledger for registering assets, and/or an accounting system for transacting them on a global scale that can comprise all forms of assets held by parties worldwide. The database network system may be used for any form of asset registry, inventory, and exchange, comprising every area of finance, economics, money, hard assets (e.g., physical properties), intangible assets such as votes, ideas, reputation, intention, health data, personal data, media, and more.

In some cases, the database network system may be resistant to modification of data, or be immutable. The database network system may be an open and distributed ledger that can record transactions between two or more parties efficiently and in a verifiable way. Transactions may be recorded permanently. A blockchain may be managed by a network collectively adhering to a protocol (such as an algorithm) for inter-node communication and validation of new blocks. In some cases, once the data is recorded, the data in a given block cannot be altered retroactively without alteration of all subsequent blocks. Designs may be optimized such that they facilitate robust workflows where participants' uncertainty regarding data security may be marginal. The use of the database network system may remove the characteristic of infinite reproducibility from a digital asset. In some cases, it may confirm that each unit of value was transferred only once, solving the double-spending problem. For example, the transactions may be non-recursive and may not be prone to be repeated once validated in a block. The database network system may comprise a value-exchange protocol. The database network system may be capable of maintaining title rights, such that when properly set up to detail the exchange agreement, it may provide a record that compels offer and acceptance.

In some examples, the database network system may be public. Participants may be allowed to verify and audit transactions independently and relatively inexpensively. The database network system may be managed autonomously using a network and a distributed timestamping server. They may be authenticated by mass collaboration powered by collective self-interest.

Blocks may hold batches of valid transactions that may be hashed and encoded into a structure. Each block may include the cryptographic hash of the prior block in the database network system, which may link them. This process may be performed iteratively (e.g., repetitively, redundantly). The iterative process may confirm the integrity of the previous block, all the way back to the genesis block.

In some cases, separate blocks may be produced concurrently, creating a temporary fork. In addition to a secure hash-based history, the database network system may have a specified algorithm for scoring different versions of the history so that one with a higher score can be selected over others. Blocks not selected for inclusion in the chain may be called orphan blocks.

The database network system may comprise a plurality of nodes. A node may be an entity, such as a machine (e.g., computer or another processing device) which is connected to the blockchain network. A machine may be controlled by a user. A machine may be controlled by another machine. A node may comprise a computer system described elsewhere herein. Each node may be capable of performing or configured to perform the task of validating and relaying transactions in the network. In some instances, each node may have a copy of the network system, which in some cases, may be downloaded once a user joins the network. In some cases, the download may be automatic.

The database network system may facilitate decentralized data distribution. Data may be stored across the entire network. By storing data across the entire network, the database network system may help decrease a number of risks that may otherwise be associated with data being held centrally, such as in an example centralized network. In some cases, the decentralized database network system may use ad-hoc message passing and/or distributed networking. The database network system may lack centralized points of vulnerability that malicious third parties can exploit; likewise, in some cases, there may be no central point of failure. In some cases, the database network system may be more secure against vulnerabilities and external attacks compared to a centralized network. In some cases, the database network system provided herein may be more secure against external attacks compared to other decentralized networks such as other blockchain networks.

In some embodiments, the tokens may be issued (or minted) and burned (or redeemed) within the engagement marketplace 465 that utilizes the distributed technology network. In some embodiments, a digital wallet is automatically created for a user when they engage with the system described herein. The minting of the tokens may be tied to a user providing a story to the insight engine 471 (based on, for example, the number of stories, the length of stories, and/or the content of stories), and the redemption of the token may be tied to the user using the token for a purchase. In some embodiments, the marketplace matches users to companies, brands, and/or products based on a well-being index and/or well-being insights for the user generated by the technologies described herein.

In some embodiments, the tokens can include non-fungible tokens (NFTs). NFTs are generally created using the same type of tools generated for cryptocurrencies. NFTs can be built on a distributed ledger network, such as the one built for the engagement marketplace 465. An NFT is a unique identifier that cannot be copied, substituted, or subdivided, and are recorded on the blockchain in order to certify authenticity and ownership. The ownership of an NFT is recorded in the blockchain and can be transferred by the owner, allowing NFTs to be sold and traded. NFTs typically include references to digital files such as photos, videos, and audio. Because NFTs are uniquely identifiable assets, they differ from cryptocurrencies, which are fungible.

In some embodiments, the stories that are recorded by the user can be encoded as an NFT. In some embodiments, the tokens that are generated for the engagement marketplace 465 can be redeemed for items that include NFTs. In some embodiments, the artwork and/or music (e.g., songs) can be encoded as an NFT.

### Machine Learning Models

In some embodiments, the insight engine 471 can be used to support the well-being of its users. The insight engine 471 can be deployed on the computer system described in FIGS. 1-3. In some embodiments, the insight engine 471 can be deployed to derive one or more insights regarding the user based on one or more stories that the user records. Generally, the insight engine 471 can deploy one or more machine learning models that can receive as input text of the stories and then predict insights for the user. In some embodiments, the insight engine 471 can use the one or more derived insights and generate one or more recommended next actions for the user.

In some embodiments, the insight engine 471 can be used to support the well-being of a group and/or population of users. In further embodiments, the methods described herein comprise a step of identifying or defining a group or population. In some cases, groups or populations are identified or defined manually. In other cases, groups or populations are identified or defined automatically by one or more of the processes and/or models described herein. In this disclosure, any function or feature that is described with respect to a group or population can be used for an individual.

Groups or populations include, by way of non-limiting examples, members of an organization (such as a team, company, school, college, or university), individuals involved in a conflict, individuals involved in a natural disaster, and individuals occupying, from, or passing through a geographic region. In some cases, a group or population is defined by factors affecting well-being of the members that are shared by, in common to, or similar among, the members of the group or population. In some embodiments, the insight engine 471 can be deployed to derive one or more insights regarding the group and/or population of users based on one or more combined stories that the users of the group and/or population record.

In some embodiments, the methods described herein comprise a step of determining or authoring one or more prompts for a group or population. In some cases, prompts for a group or population are identified or authored manually. In other cases, prompts for a group or population are identified or defined automatically by one or more of the processes and/or models described herein. In some embodiments, groups or populations are organized, prompts for groups or populations are coordinated, and/or stories from groups or populations are identified by providing an identifier, such as a URL, QR code, or the like, to members of the group.

Generally, the insight engine 471 can deploy one or more machine learning models that can receive as input text of the stories, combine the stories, and then predict insights for the group and/or population. In further embodiments, stories are combined by using statistical or probabilistic techniques. For example, in some cases, elements are identified in story media and each element is weighted based on, by way of examples, how often in a story it is used, when in a story it is used, in what context in a story it is used, and in how many stories it is used. In various embodiments, group- and/or population-level insights include, by way of non-limiting examples, shared sentiments, commonalities, degree of sense of belonging, identification of rifts, schisms, and/or sub-groups, use of resources, and the like. In some embodiments, the insight engine 471 can use the one or more derived insights and generate one or more recommended next actions for the group and/or population. In some embodiments, the insight engine 471 can deploy one or more machine learning models to generate the one or more insights and/or the one or more recommended next actions.

In cases where insights and recommendations are generated for, and provided to, an individual (or their healthcare provider) based on stories they have shared, privacy and data security such as data encryption are, or course, important. In cases where insights and recommendations are generated for a group or population, based on collective stories, in some embodiments, further enhanced privacy and data security features are utilized, because the insights and recommendations may be provided to others not in the group or population or not contributing stories of their own. In further embodiments, the purpose of such enhanced privacy and data security features is to prevent data from being traced back to any particular individual in the group or population. In some embodiments, proper nouns are identified and stripped out or replaced to anonymize persons, places, and identifiable things in text generated from shared story media. In some embodiments, after translation and transcription, audio and/or video media files, which may be personally identifiable, are securely deleted.

In some embodiments, a first machine learning model can include an unstructured machine learning model that can be used to generate and/or derive the one or more insights about the user. The first machine learning model can be trained based on a training dataset that is generated by past users and/or the user currently using the insight engine 471. The training dataset for the first machine learning model can include sensor data generated from the wearable devices, stories that were recorded by users, reviews that were generated by the users based on their thoughts on various topics, answers to questions that the user provided in response to questions, and/or indications (e.g., check-ins) that the users provided of whether the users participated in one or more activities that were provided as a recommended next action.

In some embodiments, a training dataset for the one or more models may be initially created based on user recordings of one or more stories (or user-generated narratives). In some embodiments, the audio (or the audio of the video) can be transcribed, and unstructured text in the transcription can be placed into a semi-structured format via help of the prompts used to guide the user in telling each story. In some embodiments, the dataset can also be augmented to using, for example, sentence paraphrasing and existing open source models such as the Bidirectional Encoder Representations from Transformers (BERT) model.

In some embodiments, the transcribed text may be translated into a different language. For example, when the received story is in Polish, the story may be transcribed into Polish first, and then translated into English, before the text is run through an algorithm.

In some embodiments, the dataset can be labeled with one or more preset intent/topic labels by an intent classification model. For example, an intent/topic label of “messy_workspace” may be generated by a prompt and answer as follows. The prompt may be “Describe your current workspace. How often do you organize your desk?” and the answer may include “Well, I'll avoid the feeling I have right now of turning the camera around and showing what a disarray my desk is, but it is not important to me to have everything in its place.” Such intent/topic labels can be generated for a variety of topics that include emotions, goals, expectations, past traumas, qualities/traits, etc.

In some embodiments, the prompts may be tailored to specific situations. In some embodiments, if it is known that the user experienced a specific situation, the prompt may be tailored to the specific situation. In some embodiments, the user may be asked to provide information about certain aspects and/or events in their lives that can inform the insights engine 471 to generate one or more prompts that are tailored to the specific situation. For example, if the user indicates that the user fled a war in their own country, the insights engine 471 may generate a prompt that prompts the user to provide a story about the war, about how they fled, about the people they fled with, etc.

In some embodiments, active learning may occur based of feedback related to the predicted labels for fine-tuning and/or updating the intent classification model. In some embodiments, the learning can entail invoking sentence encoders to determine statistically significant portions of a sentence for generating the intent labels. In some embodiments, a logistic regression may also be used to learn and fine-tune relationships between sentences in the stories with intent labels. In some embodiments, the active learning and/or the intent classification model may use machine learning methodologies.

In some embodiments, a training dataset may be used initially to train the intent classification model to generate intent labels for the dataset. The training dataset may include stories along with intent labels. The training dataset may also include augmented data and a library of sentences that have been gathered by scraping third-party forums. The training can invoke logistic regression and also leverage existing machine learning techniques, such as BERT, for training the intent classification model. In some embodiments, the model may be evaluated and adjusted as necessary based on learning. In some embodiments, the technology described herein utilizes LIWC models. In some embodiments, the technology described herein utilizes one or more LLMs to identify, classify, and/or measure emotion and/or sentiment. In some embodiments, a suitable methodology for developing a training dataset for one or more models comprises compiling language in shared spoken-word stories, convening a panel of experts (e.g., experts in psychology, human language, etc.) to review (at the sub-sentence level) words and phrases to assign sentiment and/or emotion, and using these assignments to train the model(s). In further embodiments, the position of one or more words and/or one or more phrases within a story is considered to assign sentiment and/or emotion, wherein words and phrases at or near the apex of the story are emphasized. In still further embodiments, additional characteristics of stories, including by way of examples, intonation, pacing (words per minute and variability), volume, pauses, word choice, order of words, number of pronouns, tense of verbs, and the like are utilized to assign sentiment and/or emotion.

In some embodiments, the insight engine 471 can run a sentiment analysis algorithm (e.g., ROBERTa) to extract one or more sentiments/emotions and/or opinions of the user attached to the topic associated with the story. The one or more sentiments/emotions and/or opinions can include a personality metric, a personal theme, a speech pattern, a stress metric, a user motivation, and/or an emotional well-being metric. The output of the sentiment analysis algorithm can include a sentiment polarity score on a scale of −1 for negative to +1 for positive. In some embodiments, a story comprises spoken-word audio of about 1 minute to about 3 minutes. In further embodiments, a story of spoken-word audio about 90 seconds to about 150 seconds in duration, is advantageous for identifying, classifying, and/or measuring emotion and/or sentiment. In still further embodiments, the position of one or more words and/or one or more phrases within the story is utilized, wherein words and phrases at or near the “apex” or “climax” of the story are up-weighted. In some embodiments, additional characteristics of a story, including by way of examples, intonation, pacing (words per minute and variability), volume, pauses, word choice, order of words, number of pronouns, tense of verbs, and the like are utilized to identify, classify, and/or measure emotion and/or sentiment.

In some embodiments, a machine learning model can be deployed to generate a summary of the story. In some embodiments, a machine learning model can be deployed to “fingerprint” one or more stories to identify a user and/or prevent impersonation or “spoofing” of a user's identity. In further embodiments, verification and authentication of a user's identity via ML fingerprinting of story audio can prevent fraud in an associated marketplace that matches users to companies, brands, and/or products based on a well-being index or well-being insights for the user.

In some embodiments, the user can wear one or more devices that can generated sensor data. The sensor data can be generated from any device that includes a sensor on the user and/or near the user. For example, the sensor data can include data generated from a wearable device that can detect heart rates, exercise metrics, oxygen levels, sleep patterns, activity data, etc.

In some embodiments, the intent labels output by the intent classification model, sentiment polarity scores from sentiment analysis, and Likert scores can be used to compute wellness dimension scores for the dimension to which the story relates.

In some embodiments, wellness dimension scores may be used to compute personality insights (or insights) in the form of personality scores. In some embodiments, one or more of the wellness dimension scores can be used to compute the one or more insights. In some embodiments, the insights can include sentiment, intent, patterns, beliefs, and motivations of the user whose stories were analyzed.

In some embodiments, the sentiment analysis is performed for a group of people, or a population, instead of one individual. In some embodiments, the group of people have gone through a similar experience. For example, the group of people may have experienced a war in their own country, an earthquake, a fire, a tsunami, a natural disaster, etc. In some embodiments, all of the narratives of the group of people can be compiled, or combined, into one narrative and analyzed together as one narrative. Then group insights and group recommended next actions can be derived that can be generally applied to the group of people.

In some embodiments, a second machine learning model can include a structured machine learning model that can be used to generate one or more recommended next actions. For example, the user can be provided a recommendation to go for a walk, take a vacation, read a book, talk to a wellness professional, etc. The second machine learning model can generate one or more recommended next actions based on the user's input and/or stories that were used in the first machine learning model and/or the one or more insights generated from the first machine learning model.

In some embodiments, the insights may be provided to the user, and next actions relating to skills, tools, and content, may be recommended to the user based on such insights. In some embodiments, a supervised learning model can be used to receive the insights as input and output one or more recommended next actions.

Because well-being, the factors that can affect well-being, and other conditions change over time, in some embodiments, analyses described herein are performed one than once, on an ad hoc basis, and/or periodically (e.g., daily, weekly, monthly, etc.) to generate time-series data. In further embodiments, the time-series data is stored in a time-series database (TSDB), such as, by way of examples, MongoDB, Prometheus, Amazon Timestream, Apache Druid, and the like. In some embodiments, the time-series data is used to calculate a change or difference in sentiment, intent, well-being, insights, and the like, over time. In some embodiments, the time-series data is used to conduct benchmarking of measures of sentiment, intent, well-being, insights, and the like, against historical datasets.

### First Exemplary Embodiment

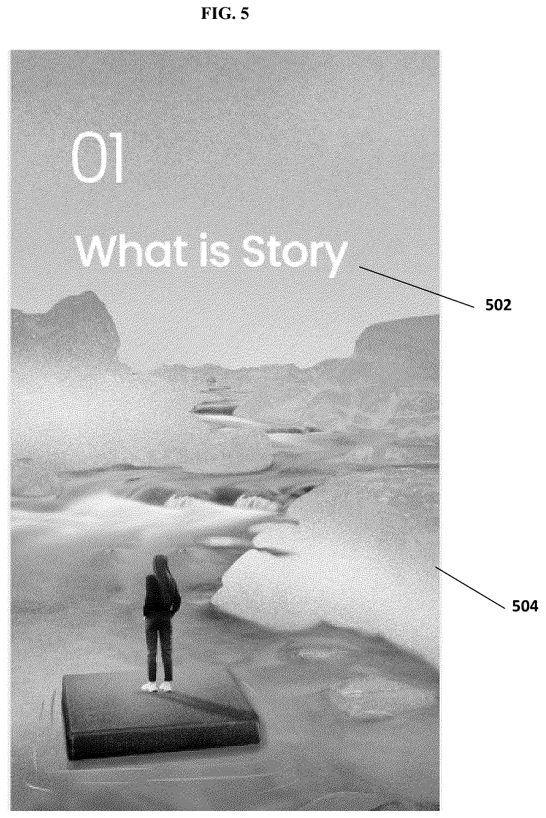

Referring to FIGS. 5-19, various non-limiting examples of GUIs for a recording studio module on the user application 405 allowing a user to record an unstructured user-generated narrative are shown. Although the examples shown in FIGS. 5-19 show how the user application 405 can be displayed in a cell phone, embodiments are not limited thereto, and the user application 405 can be launched on any electronic device, such as a tablet device, a laptop computer, a desktop computer, wearable device, etc. Furthermore, the text, font, colors, design, look and feel and other aspects of the examples shown in the GUI can vary, depending on embodiments.

FIG. 5 shows a non-limiting example of a GUI for a recording studio module allowing a user to record an unstructured user-generated narrative; in this case, a GUI displaying a first introduction page of a first topic of what a story is. The first introduction page may include a title text 502 that introduces the user to what the next one or more pages will be about. For example, the title text 502 can show “What is Story,” which can cue the user to describing what a “story” is and how to share the story to the user application 405. The introduction page may also include an image 504 that visually symbolizes and/or describes the title text 502. For example, in FIG. 5, the image 504 shows a person standing on a platform that is flowing through a stream, which can symbolize a person's experience in walking or flowing through a story. Such an image can help the user visualize and/or think of the story they want to convey. In some embodiments, the image 504 may include a video and/or multiple images.

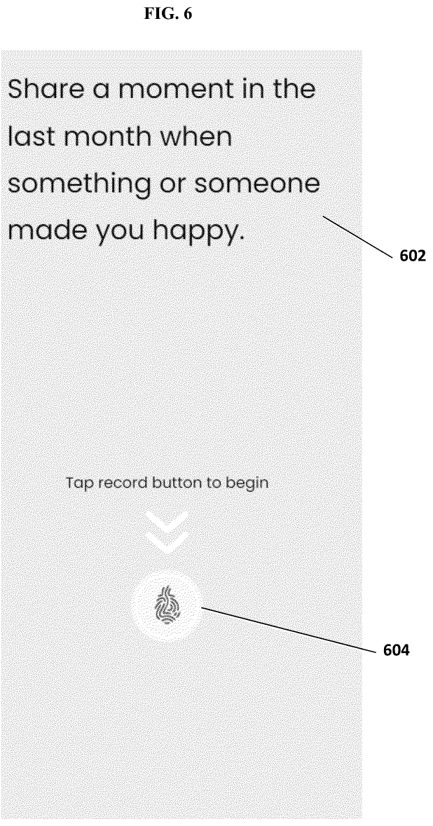

FIG. 6 shows a non-limiting example of a GUI for a recording studio module allowing a user to record an unstructured user-generated narrative; in this case, a GUI displaying a prompt page for the first topic. Generally, the prompt page can prompt the user to share a story or a moment. The prompt page may include a prompt 602 that can ask the user to share a moment in the last month when the user was happy. The prompt page can also include a button 604 that the user can press to begin recording the audio and/or video of the user who can share the moment. The button 604 can be displayed in a first color or a first combination of colors. In some embodiments, the user may initiate the recording by tapping the button 604. In some embodiments, the user may record while pressing down the button 604. In some embodiments, the prompt page can also be designed to ask the user to provide any other input such as swiping, dragging, etc.

In some embodiments, the prompt 602 can be different so that the prompt 602 can invoke and/or trigger a different response from the user, while still allowing the user to share a story or moment related to a general topic. In some embodiments, the general topic may be high level thoughts or emotions that the user may have at the time of using the user application 405. For example, the prompt 602 may prompt the user to share something that made the user excited, that made the user sad, that made the user reflective, etc. One of ordinary skill will recognize that the prompts displayed on prompt 602 can vary widely, in accordance with embodiments. In some embodiments, the stories collected from responses may be collected and analyzed to obtain more accurate insights regarding the user.

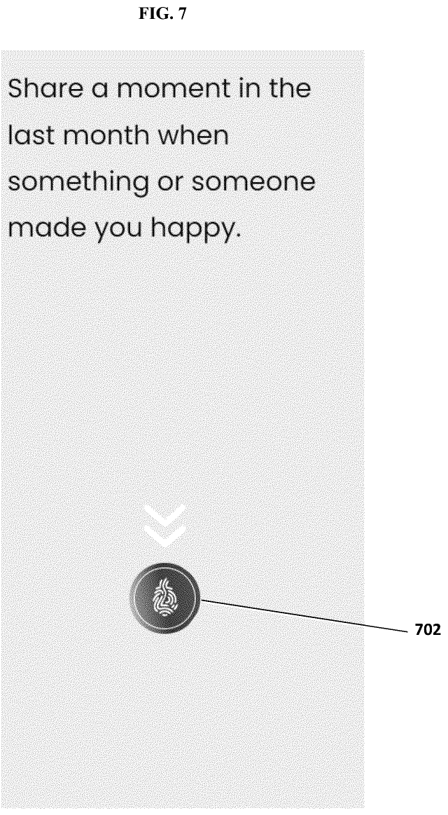

FIG. 7 shows a non-limiting example of a GUI for a recording studio module allowing a user to record an unstructured user-generated narrative; in this case, a GUI displaying a recording page for the first topic. The recording page can indicate to the user that user application 405 is recording the user's story or moment. The user can share their story and/or moment, by speaking, which is a response to the prompt 602. The recording page may include the prompt 602 that was displayed in the prompt page to remind the user what the user is sharing. The recording page may also indicate by the recording indicator 702 that the user application 405 is recording what the user is sharing. The recording indicator 702 may include a color different than the button 604 of FIG. 6. In some embodiments, the user application 405 may turn on the camera on the mobile device and display the user on the screen. In some embodiments, the user application 405 may record a video of the user sharing the story or moment. In some embodiments, the user application 405 may record the audio only. In some embodiments, the user may tap the recording indicator to stop the recording. In some embodiments, the user may tap the recording indicator again to continue recording. In some embodiments, when the user has completed the recording, the user may swipe up or sideways such that the user application 405 may display a new page.

In some embodiments, there may be multiple prompts and recordings for each topic. For example, the user may be prompted to share something that made the user happy in the last month and then the user may record the response. The user may then be prompted to share something that made the use sad in the last month and then the user may record the response. In some embodiments, the user application 405 may share the multiple prompts in sequence (e.g., one prompt per page) so that the user can focus on the given prompt when recording the user's response. The embodiments are not limited to how many prompts and recordings there are for each general topic.

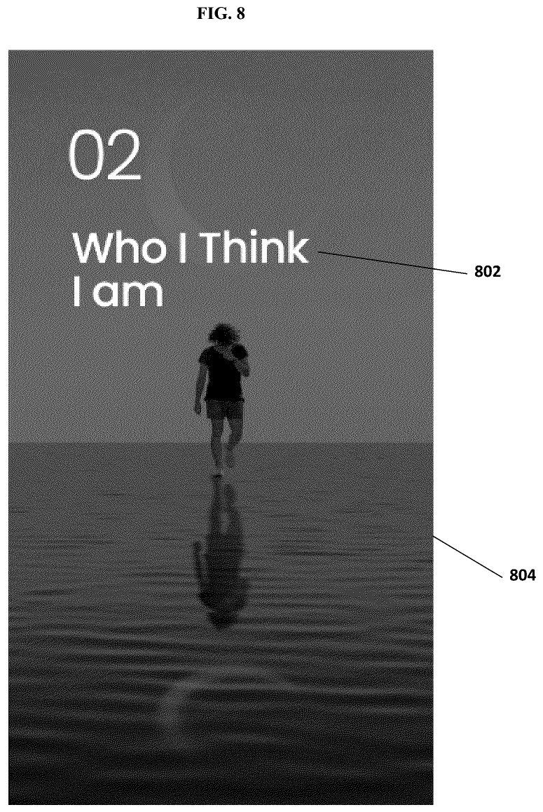

FIG. 8 shows a non-limiting example of a GUI for a recording studio module allowing a user to record an unstructured user-generated narrative; in this case, a GUI displaying a second introduction page of a second topic of describing the user. The second introduction page may include a title text 802 that introduces the user to what the next one or more pages will be about. For example, the title text 802 can show “Who I think I am,” which can cue the user to describing oneself to the user application 405. The second introduction page may also include an image 804 that visually symbolizes and/or describes the title text 802. For example, in FIG. 8, the image 804 shows a person walking over a body of water with ripples coming from the place the person is, which can symbolize a person's own journey and how they became who they are at the time of sharing the recording. In some embodiments, the image 804 may include a video and/or multiple images.

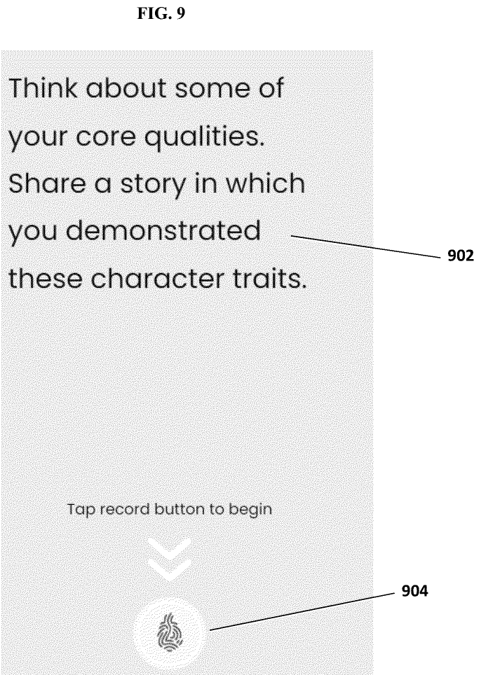

FIG. 9 shows a non-limiting example of a GUI for a recording studio module allowing a user to record an unstructured user-generated narrative; in this case, a GUI displaying a prompt page of the second topic. Generally, the prompt page can prompt the user to share a story or a moment. The prompt page may include a prompt 802 that can ask the user to think about some of the user's own core qualities and share a story that demonstrates these traits. The prompt page can also include a button 904 that the user can press to begin recording the audio and/or video of the user who can share the moment. The button 904 can be displayed in a first color or a first combination of colors. In some embodiments, the user may initiate the recording by tapping the button 904. In some embodiments, the user may record while pressing down the button 904. In some embodiments, the prompt page can also be designed to ask the user to provide any other input such as swiping, dragging, etc.

In some embodiments, the prompt 902 can be different so that the prompt 902 can invoke and/or trigger a different response from the user, while still allowing the user to share a story or moment related to a general topic. In some embodiments, the general topic may be general qualities and traits that the user has about oneself. For example, the prompt 902 may prompt the user to share a story that demonstrates traits about the user, a story that demonstrates the user's shortcomings, a story about what others have shared about the user, etc. One of ordinary skill will recognize that the prompts displayed on prompt 902 can vary widely, in accordance with embodiments. In some embodiments, the stories collected from responses may be collected and analyzed to obtain more accurate insights regarding the user.

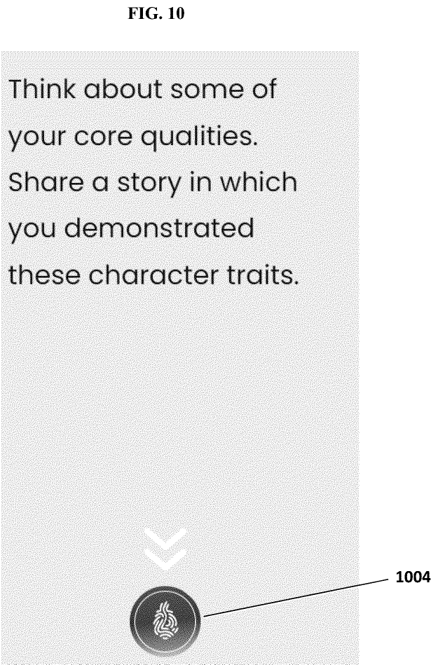

FIG. 10 shows a non-limiting example of a GUI for a recording studio module allowing a user to record an unstructured user-generated narrative; in this case, a GUI displaying a recording page of the second topic. The recording page can indicate to the user that user application 405 is recording the user's story or moment. The user can share their story and/or moment, by speaking, which is a response to the prompt 902. The recording page may include the prompt 902 that was displayed in the prompt page to remind the user what the user is sharing. The recording page may also indicate by the recording indicator 1004 that the user application 405 is recording what the user is sharing. The recording indicator 1004 may include a color different than the button 904 of FIG. 9. In some embodiments, the user application 405 may turn on the camera on the mobile device and display the user on the screen. In some embodiments, the user application 405 may record a video of the user sharing the story or moment. In some embodiments, the user application 405 may record the audio only. In some embodiments, the user may tap the recording indicator to stop the recording. In some embodiments, the user may tap the recording indicator again to continue recording. In some embodiments, when the user has completed the recording, the user may swipe up or sideways such that the user application 405 may display a new page.

In some embodiments, there may be multiple prompts and recordings for each topic. For example, the user may be prompted to share a story about the user's qualities that make the user attractive to others and then the user may record the response. The user may then be prompted to share a story about the user's qualities that the user is working on and then the user may record the response. In some embodiments, the user application 405 may share the multiple prompts in sequence (e.g., one prompt per page) so that the user can focus on the given prompt when recording the user's response. The embodiments are not limited to how many prompts and recordings there are for each general topic.

FIG. 11 shows a non-limiting example of a GUI for a recording studio module allowing a user to record an unstructured user-generated narrative; in this case, a GUI displaying a third introduction page of the third topic of describing the user's family. The third introduction page may include a title text 1102 that introduces the user to what the next one or more pages will be about. For example, the title text 1102 can show “Your Family Story,” which can cue the user to describing the user's family to the user application 405. The third introduction page may also include an image 1104 that visually symbolizes and/or describes the title text 1102. For example, in FIG. 11, the image 1104 shows a person standing on a hill and looking up to a silhouette of the person with several other people and everyone holding hands, which can symbolize a person's own family and how that family has impacted the person. In some embodiments, the image 1104 may include a video and/or multiple images.

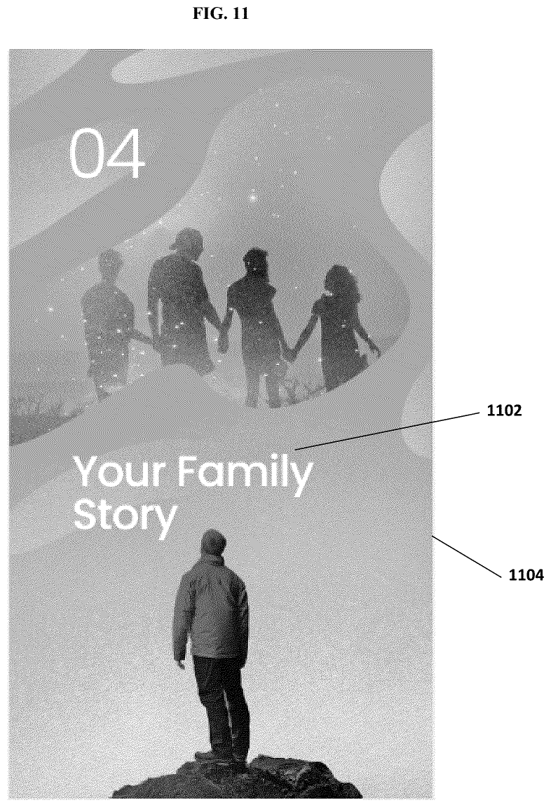

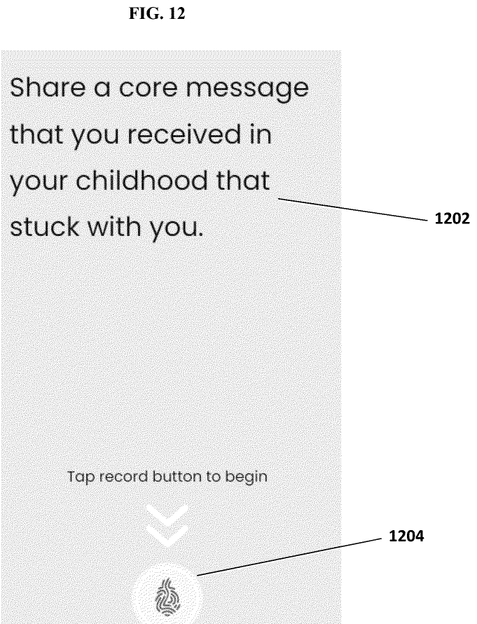

FIG. 12 shows a non-limiting example of a GUI for a recording studio module allowing a user to record an unstructured user-generated narrative; in this case, a GUI displaying a prompt page of the third topic. Generally, the prompt page can prompt the user to share a story or a moment. The prompt page may include a prompt 1202 that can ask the user to share a core message that you received in the user's childhood that stuck with you. The prompt page can also include a button 1204 that the user can press to begin recording the audio and/or video of the user who can share the moment. The button 1204 can be displayed in a first color or a first combination of colors. In some embodiments, the user may initiate the recording by tapping the button 1204. In some embodiments, the user may record while pressing down the button 1204. In some embodiments, the prompt page can also be designed to ask the user to provide any other input such as swiping, dragging, etc.

In some embodiments, the prompt 1202 can be different so that the prompt 1202 can invoke and/or trigger a different response from the user, while still allowing the user to share a story or moment related to a general topic. In some embodiments, the general topic may be the user's childhood and upbringing. For example, the prompt 1202 may prompt the user to share a story about the user's siblings, about the user's parents, about the user's relatives, about a time that a sibling upset the user, about a time that the parent reprimanded the user, etc. One of ordinary skill will recognize that the prompts displayed on prompt 1102 can vary widely, in accordance with embodiments. In some embodiments, the stories collected from responses may be collected and analyzed to obtain more accurate insights regarding the user.

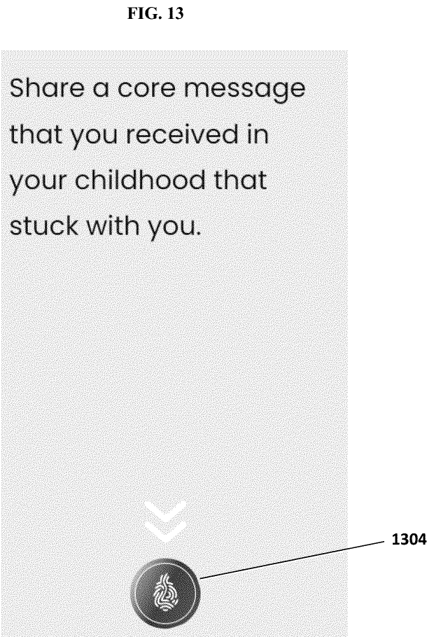

FIG. 13 shows a non-limiting example of a GUI for a recording studio module allowing a user to record an unstructured user-generated narrative; in this case, a GUI displaying a recording page of the third topic. The recording page can indicate to the user that user application 405 is recording the user's story or moment. The user can share their story and/or moment, by speaking, which is a response to the prompt 1202. The recording page may include the prompt 1302 that was displayed in the prompt page to remind the user what the user is sharing. The recording page may also indicate by the recording indicator 1304 that the user application 405 is recording what the user is sharing. The recording indicator 1304 may include a color different than the button 1204 of FIG. 12. In some embodiments, the user application 405 may turn on the camera on the mobile device and display the user on the screen. In some embodiments, the user application 405 may record a video of the user sharing the story or moment. In some embodiments, the user application 405 may record the audio only. In some embodiments, the user may tap the recording indicator to stop the recording. In some embodiments, the user may tap the recording indicator again to continue recording. In some embodiments, when the user has completed the recording, the user may swipe up or sideways such that the user application 405 may display a new page.

In some embodiments, there may be multiple prompts and recordings for each topic. For example, the user may be prompted to share a story about a time when the user's sibling made him happy and then the user may record the response. The user may then be prompted to share a story about a time when the user's family went on a memorable trip and then the user may record the response. In some embodiments, the user application 405 may share the multiple prompts in sequence (e.g., one prompt per page) so that the user can focus on the given prompt when recording the user's response. The embodiments are not limited to how many prompts and recordings there are for each general topic.

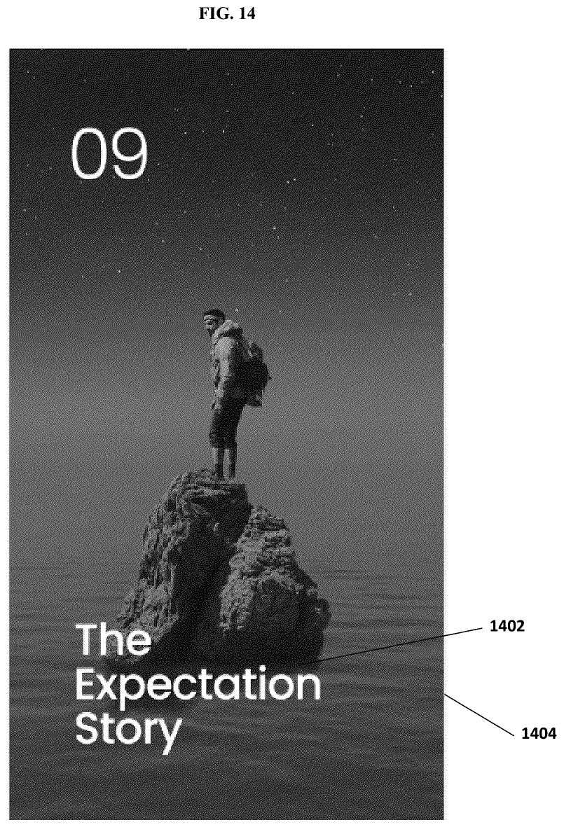

FIG. 14 shows a non-limiting example of a GUI for a recording studio module allowing a user to record an unstructured user-generated narrative; in this case, a GUI displaying a fourth introduction page of a fourth topic of describing the expectations. The fourth introduction page may include a title text 1402 that introduces the user to what the next one or more pages will be about. For example, the title text 1402 can show “The Expectation Story,” which can cue the user to describing the user's expectations in life to the user application 405. The fourth introduction page may also include an image 1404 that visually symbolizes and/or describes the title text 1402. For example, in FIG. 14, the image 1404 shows a person standing on a rock in a body of water and carrying a backpack, which can symbolize a person's own accomplishments in the past and other goals they hope to accomplish in the future. In some embodiments, the image 1404 may include a video and/or multiple images.

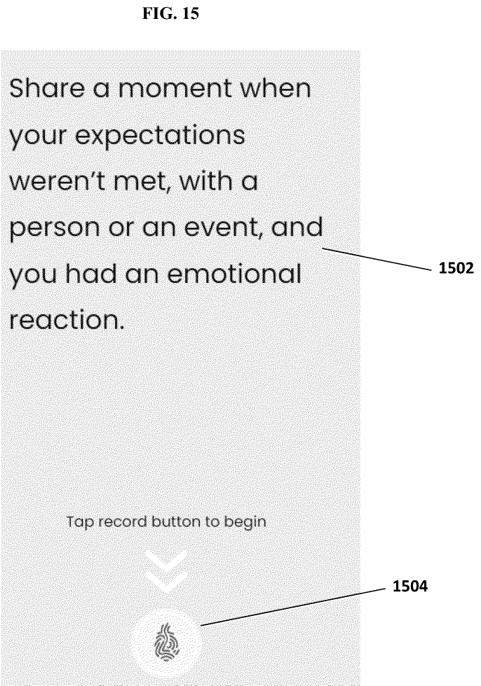

FIG. 15 shows a non-limiting example of a GUI for a recording studio module allowing a user to record an unstructured user-generated narrative; in this case, a GUI displaying a prompt page of the fourth topic. Generally, the prompt page can prompt the user to share a story or a moment. The prompt page may include a prompt 1502 that can ask the user to share a moment when the user's expectations were not met, with a person or an event, and the user had an emotional reaction. The prompt page can also include a button 1504 that the user can press to begin recording the audio and/or video of the user who can share the moment. The button 1504 can be displayed in a first color or a first combination of colors. In some embodiments, the user may initiate the recording by tapping the button 1504. In some embodiments, the user may record while pressing down the button 1504. In some embodiments, the prompt page can also be designed to ask the user to provide any other input such as swiping, dragging, etc.

In some embodiments, the prompt 1502 can be different so that the prompt 1502 can invoke and/or trigger a different response from the user, while still allowing the user to share a story or moment related to a general topic. In some embodiments, the general topic may be the user's expectations and goals. For example, the prompt 1502 may prompt the user to share a story about a time when the user was able to accomplish a goal, about a time the user failed at a task, about a time the user overcome an obstacle, etc. One of ordinary skill will recognize that the prompts displayed on prompt 1502 can vary widely, in accordance with embodiments. In some embodiments, the stories collected from responses may be collected and analyzed to obtain more accurate insights regarding the user.

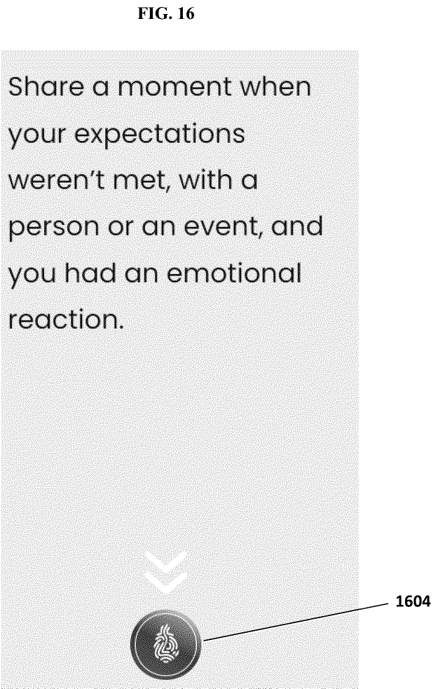

FIG. 16 shows a non-limiting example of a GUI for a recording studio module allowing a user to record an unstructured user-generated narrative; in this case, a GUI displaying a recording page of the fourth topic. The recording page can indicate to the user that user application 405 is recording the user's story or moment. The user can share their story and/or moment, by speaking, which is a response to the prompt 1502. The recording page may include the prompt 1502 that was displayed in the prompt page to remind the user what the user is sharing. The recording page may also indicate by the recording indicator 1604 that the user application 405 is recording what the user is sharing. The recording indicator 1604 may include a color different than the button 1604 of FIG. 15. In some embodiments, the user application 405 may turn on the camera on the mobile device and display the user on the screen. In some embodiments, the user application 405 may record a video of the user sharing the story or moment. In some embodiments, the user application 405 may record the audio only. In some embodiments, the user may tap the recording indicator to stop the recording. In some embodiments, the user may tap the recording indicator again to continue recording. In some embodiments, when the user has completed the recording, the user may swipe up or sideways such that the user application 405 may display a new page.

In some embodiments, there may be multiple prompts and recordings for each topic. For example, the user may be prompted to share a story about a time when the user accomplished a goal and then the user may record the response. The user may then be prompted to share a story about a time when the user accomplished only a portion of the user's goals and then the user may record the response. In some embodiments, the user application 405 may share the multiple prompts in sequence (e.g., one prompt per page) so that the user can focus on the given prompt when recording the user's response. The embodiments are not limited to how many prompts and recordings there are for each general topic.

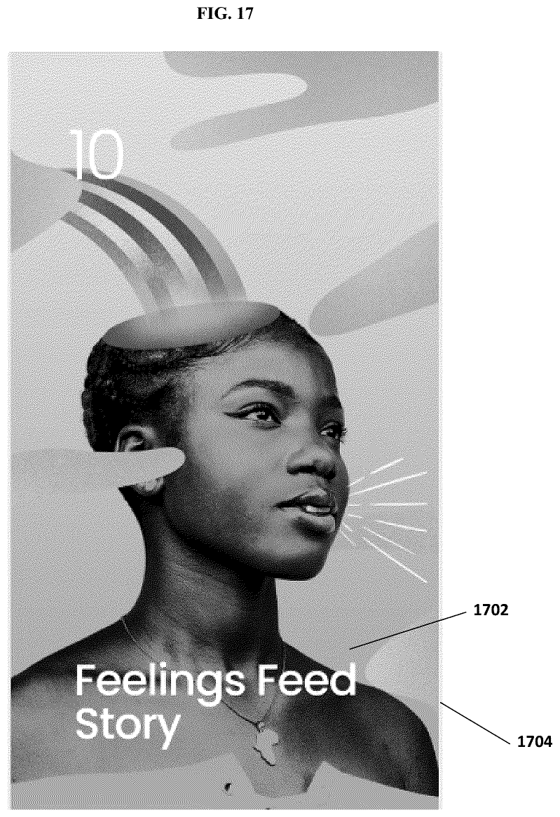

FIG. 17 shows a non-limiting example of a GUI for a recording studio module allowing a user to record an unstructured user-generated narrative; in this case, a GUI displaying a fifth introduction page of a fifth topic of describing the user's feelings. The fifth introduction page may include a title text 1702 that introduces the user to what the next one or more pages will be about. For example, the title text 1702 can show “Feelings Feed Story,” which can cue the user to describing the user's feelings to the user application 405. The fifth introduction page may also include an image 1704 that visually symbolizes and/or describes the title text 1702. For example, in FIG. 17, the image 1704 shows a person whose head is connected to a colorful rainbow and lines coming out of the mouth, which can symbolize a person's own varied feelings and emotions and the person verbalizing those feelings and emotions. In some embodiments, the image 1704 may include a video and/or multiple images.

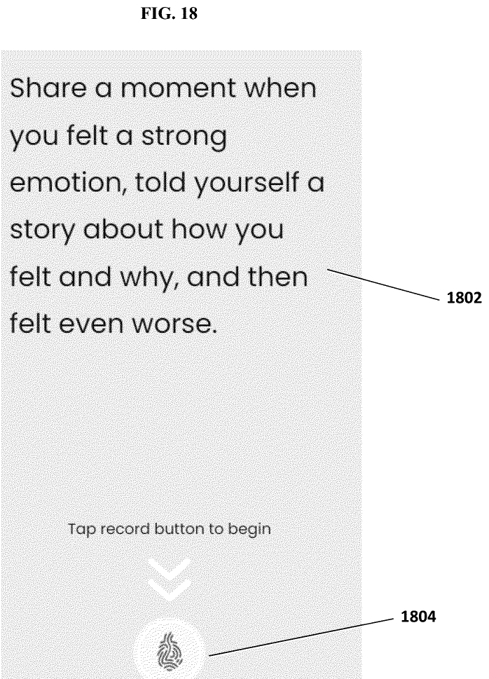

FIG. 18 shows a non-limiting example of a GUI for a recording studio module allowing a user to record an unstructured user-generated narrative; in this case, a GUI displaying a prompt page of the fourth topic. Generally, the prompt page can prompt the user to share a story or a moment. The prompt page may include a prompt 1802 that can ask the user to share a moment when the user felt a strong emotion, told oneself a story about how the user felt and why, and then felt even worse. The prompt page can also include a button 1804 that the user can press to begin recording the audio and/or video of the user who can share the moment. The button 1804 can be displayed in a first color or a first combination of colors. In some embodiments, the user may initiate the recording by tapping the button 1804. In some embodiments, the user may record while pressing down the button 1804. In some embodiments, the prompt page can also be designed to ask the user to provide any other input such as swiping, dragging, etc.

In some embodiments, the prompt 1802 can be different so that the prompt 1802 can invoke and/or trigger a different response from the user, while still allowing the user to share a story or moment related to a general topic. In some embodiments, the general topic may be the user's feelings and emotions. For example, the prompt 1802 may prompt the user to share a story about a time when the user felt happy, about a time when the user felt hopeful, etc. One of ordinary skill will recognize that the prompts displayed on prompt 1802 can vary widely, in accordance with embodiments. In some embodiments, the stories collected from responses may be collected and analyzed to obtain more accurate insights regarding the user.

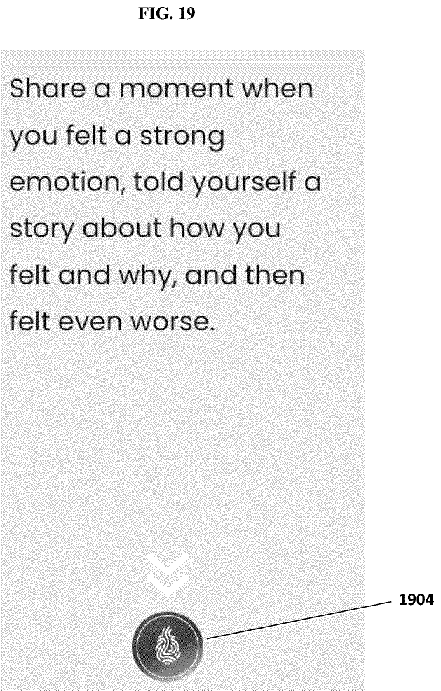

FIG. 19 shows a non-limiting example of a GUI for a recording studio module allowing a user to record an unstructured user-generated narrative; in this case, a GUI displaying a recording page of the fifth topic. The recording page can indicate to the user that user application 405 is recording the user's story or moment. The user can share their story and/or moment, by speaking, which is a response to the prompt 1802. The recording page may include the prompt 1802 that was displayed in the prompt page to remind the user what the user is sharing. The recording page may also indicate by the recording indicator 1904 that the user application 405 is recording what the user is sharing. The recording indicator 1904 may include a color different than the button 1804 of FIG. 18. In some embodiments, the user application 405 may turn on the camera on the mobile device and display the user on the screen. In some embodiments, the user application 405 may record a video of the user sharing the story or moment. In some embodiments, the user application 405 may record the audio only. In some embodiments, the user may tap the recording indicator to stop the recording. In some embodiments, the user may tap the recording indicator again to continue recording. In some embodiments, when the user has completed the recording, the user may swipe up or sideways such that the user application 405 may display a new page.

In some embodiments, there may be multiple prompts and recordings for each topic. For example, the user may be prompted to share a story about a time when the user was happy and then the user may record the response. The user may then be prompted to share a story about a time when the user was sad and then the user may record the response. In some embodiments, the user application 405 may share the multiple prompts in sequence (e.g., one prompt per page) so that the user can focus on the given prompt when recording the user's response. The embodiments are not limited to how many prompts and recordings there are for each general topic.

As described above, after the user provides one or more stories based on one or more prompts, the stories can be analyzed to generate one or more insights about the user. The analysis may include transcription of the stories, inputting the terms from the transcriptions into one or more machine learning models, and outputting the one or more insights from the models based on the models.

### Second Exemplary Embodiment

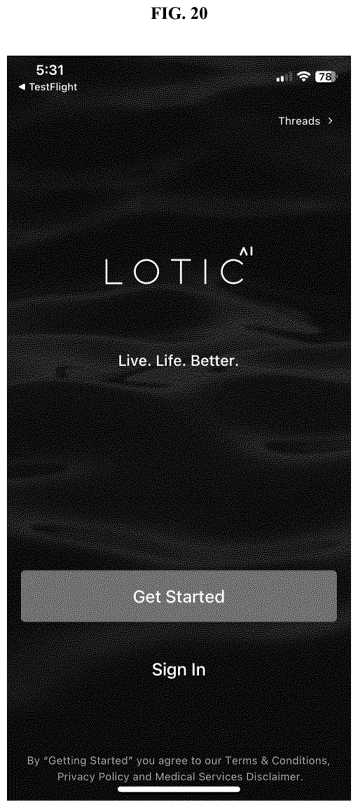

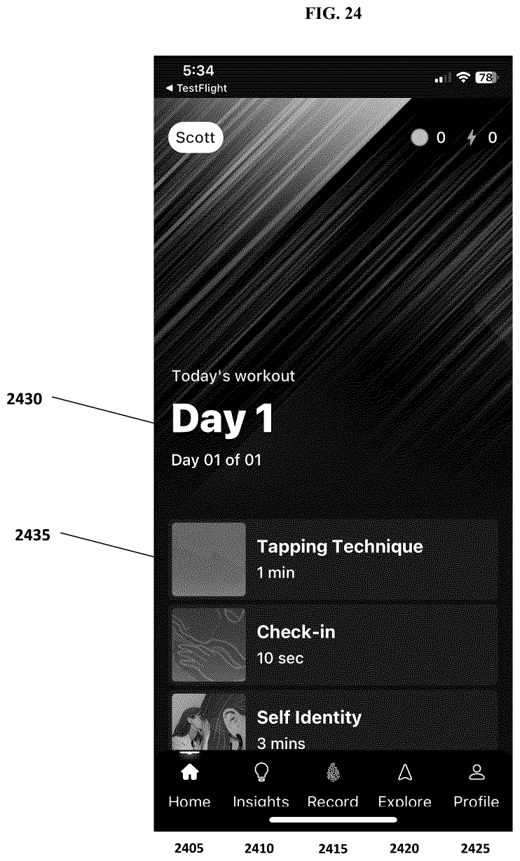

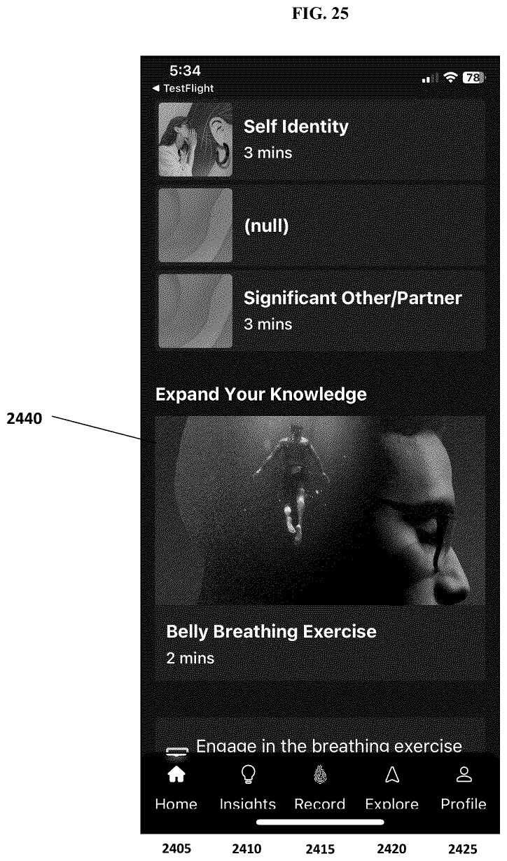

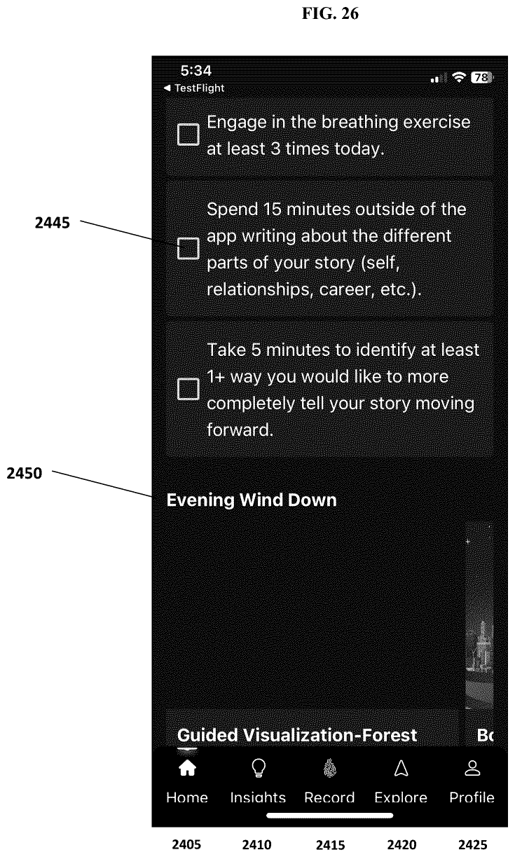

Referring to FIGS. 20-31, various non-limiting examples of GUIs for a well-being application are shown. Although the examples shown in FIGS. 20-31 show how the application can be displayed on a mobile device, embodiments are not limited thereto, and the application can be deployed to any computing device, such as a tablet device, a laptop computer, a desktop computer, a wearable device, etc. Furthermore, the text, font, colors, design, look and feel, and other aspects of the examples shown in the GUI can vary, depending on embodiments.

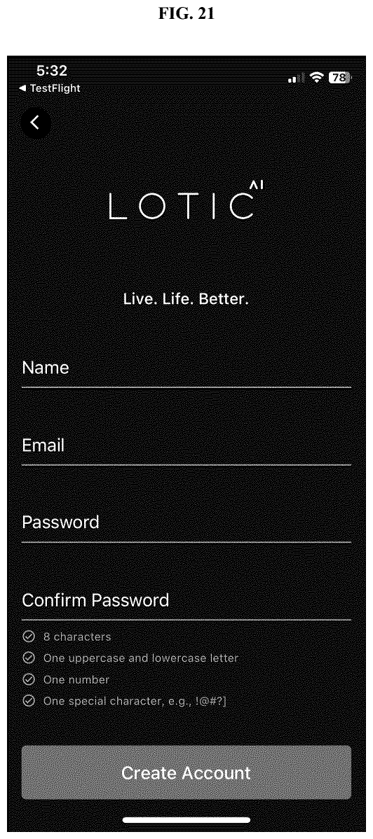

FIG. 20 shows a non-limiting example of a GUI for a well-being application; in this case, a GUI displaying a splash screen. The splash screen may include a “Get Started” button providing access to an account creation screen (see, e.g., FIG. 21) and a “Sign In” button.

FIG. 21 shows a non-limiting example of a GUI for a well-being application; in this case, a GUI displaying an account creation screen. The account creation screen may include entry fields for Name, Email, and Password, as well as a “Create Account” button.

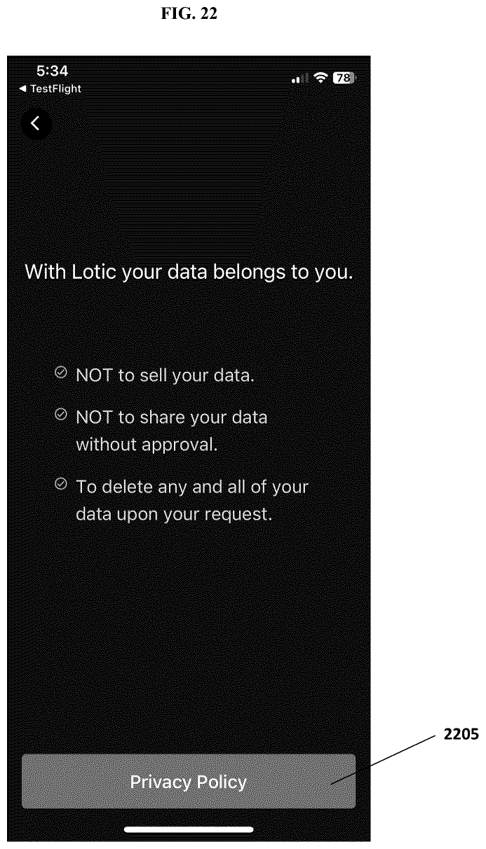

FIG. 22 shows a non-limiting example of a GUI for a well-being application; in this case, a GUI displaying a data usage information screen providing access to a privacy policy. The data usage information screen may include information pertaining to sale and sharing of user data as well as information pertaining to deletion of user date, such as a Right To Be Forgotten (RTBF). The data usage information screen may also include a “Privacy Policy” button 2205 allowing a user to access a legal privacy policy document for the application.

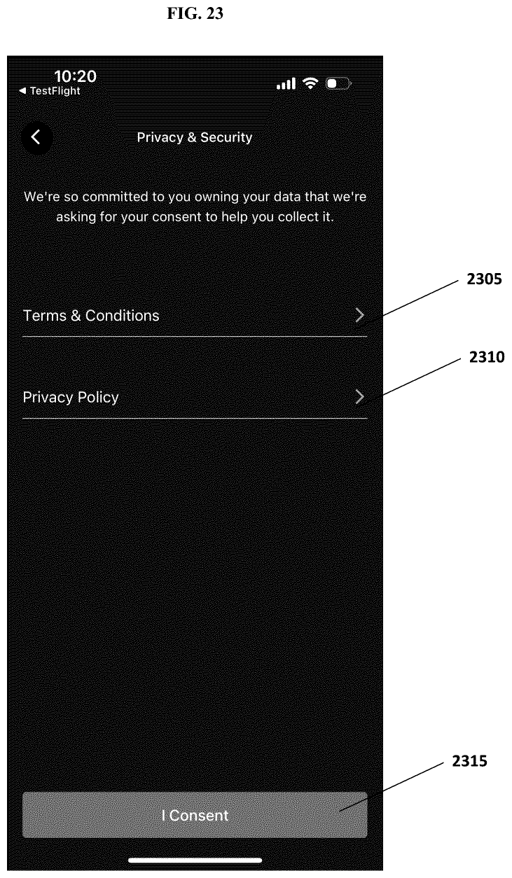

FIG. 23 shows a non-limiting example of a GUI for a well-being application; in this case, a GUI displaying a privacy and security consent screen providing access to terms & conditions and a privacy policy. The privacy and security consent screen may include links allowing a user to access legal terms & conditions 2305 and privacy policy 2310 documents as well as a “I Consent” button 2315 allowing a user to submit their acknowledgement and consent.

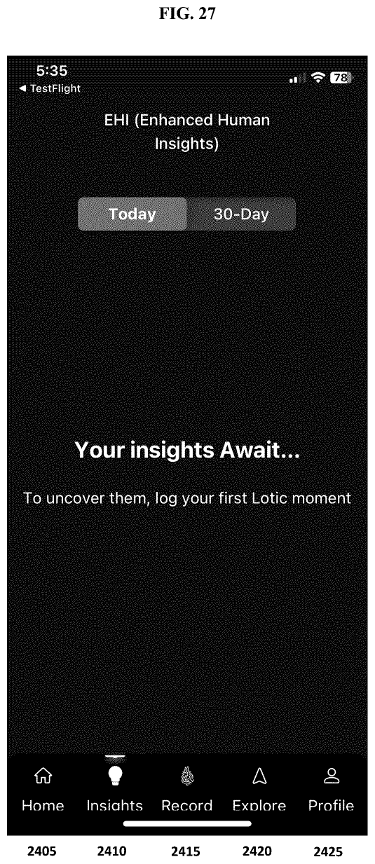

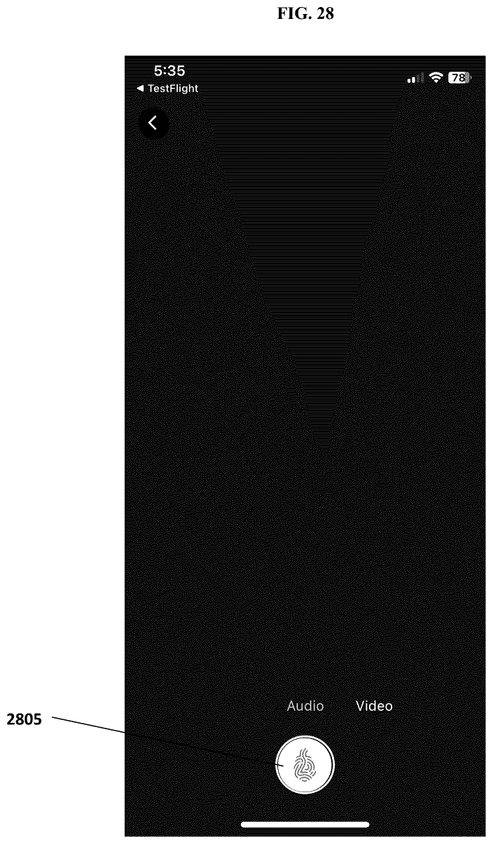

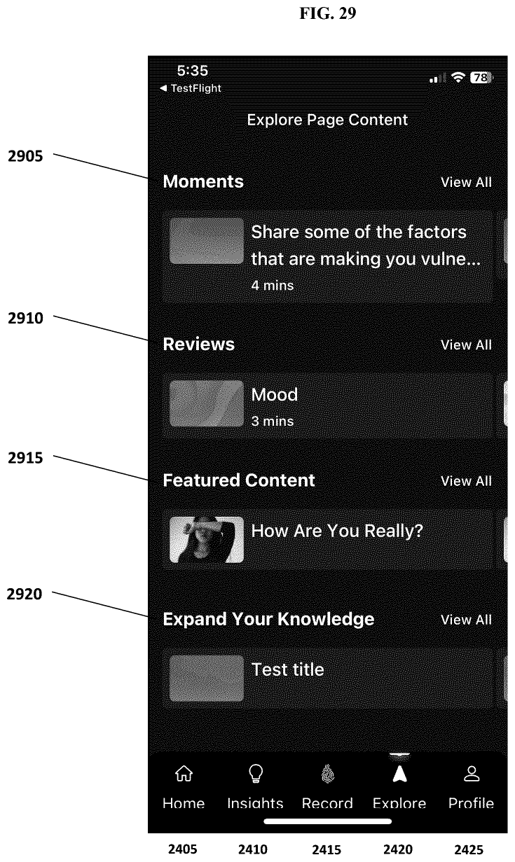

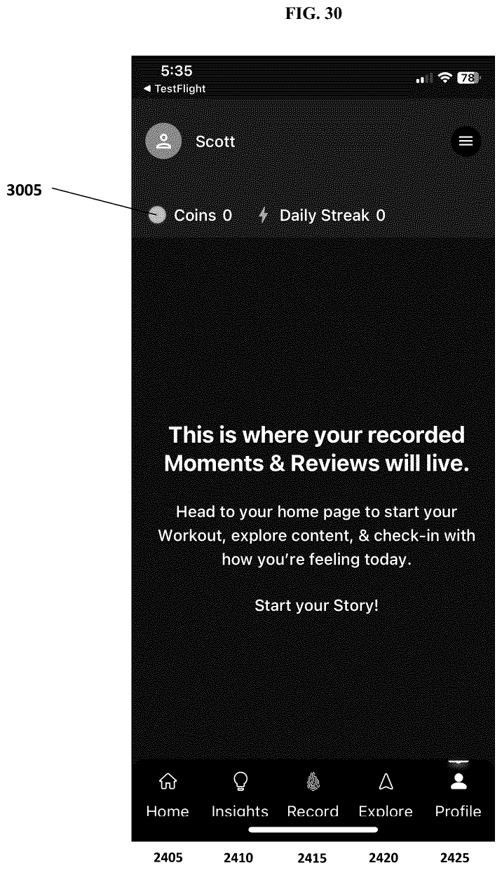

FIGS. 24-26 show a non-limiting example of a GUI for a well-being application; in this case, a GUI displaying a home screen. The home may include a set of navigation icons providing access to the home screen 2405, an insights screen 2410 (see, e.g., FIG. 27), a record screen 2415 (see, e.g., FIG. 28), an explore screen 2420 (see, e.g., FIG. 29), and a profile screen 2425 (see, e.g., FIG. 30). The home screen may include content such as a day indication 2430 and daily well-being workouts 2435 optionally including techniques, exercises, check-ins, educational materials 2440, well-being tasks 2445, and evening wind-downs 2450.

FIG. 27 shows a non-limiting example of a GUI for a well-being application; in this case, a GUI displaying an enhanced human insights (EHI) screen. The EHI screen may include one or more insights generated by the methodologies described herein.

FIG. 28 shows a non-limiting example of a GUI for a well-being application; in this case, a GUI displaying an audio and video recording studio screen. The audio and video recording studio screen may include “Audio” and “Video” selectors as well as a “Record” button 2805.

FIG. 29 shows a non-limiting example of a GUI for a well-being application; in this case, a GUI displaying an exploration screen allowing a user to browse page content. The exploration screen may provide listings of, and access to, content such as recorded user moments 2905, reviews 2910, featured content 2915 (which may be professionally curated), and educational materials 2920.

FIG. 30 shows a non-limiting example of a GUI for a well-being application; in this case, a GUI displaying a profile screen providing access to recorded moments and reviews. The profile screen may include indicators for “coin” rewards earned 3005.

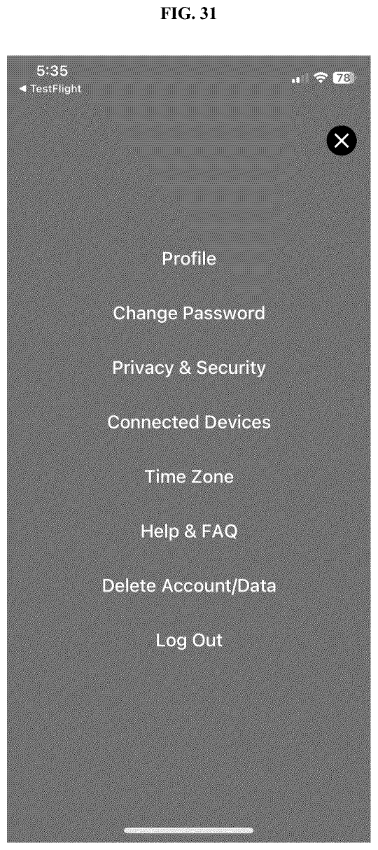

FIG. 31 shows a non-limiting example of a GUI for a well-being application; in this case, a GUI displaying a settings screen. The settings screen may provide access to settings configurations for a user, including, by way of non-limiting examples, profile settings, password settings, privacy and security settings, connected device settings, and time zone settings. The settings screen may also provide access to help screens and FAQs, tools allowing a user to delete their data and/or account, and to log-out.

### Exemplary Use Cases

### Well-being for Languishing Individuals and Populations

In various embodiments, the platforms, systems, media, and methods disclosed herein are configured for one or more use cases. In some embodiments, one or more use cases is a specific or specialized use case contemplating a particular person, group of people, or population. By way of example, there exists a middle ground between being mentally healthy and mentally ill called languishing, which is a prevalent non-clinical state. By some estimates, approximately 50% of adults are considered languishing and estimated 65% of young adults ages 18-34 are considered languishing. The longer people are in a languishing state, the greater their risk of developing mental and physical health conditions. Moreover, languishing has a compounding effect in that the longer people languish, more time is spent aimlessly searching for distraction or direction online, prompting more feelings of emptiness from social media platforms or financial strain from “quick-fix” products and services.

In some embodiments, the platforms, systems, media, and methods disclosed herein are configured for use by languishing individuals or populations. In such embodiments, the technology described herein is configured for helping users live life better by cultivating micro habits to grow and flourish, rather than providing medical treatment and/or diagnosis. The present subject matter, in some embodiments, provides a formula for helping users progress from languishing to habits of flourishing following a curiosity driven, story-focused approach, by delivering engaging strength based mental fitness training rooted in empirically based well-being science. These skills help languishing users better understand and rewrite their inner narrative, build critical resilience skills and interpret personal insights to encourage micro changes toward more positive habits. Individually tailored and linked to the future self-improvement marketplace, these insights are the impetus that connects users with additional support.

### Well-being for Students and Student Populations

By some estimates, 65% of students are considered languishing and many colleges have significant student populations at risk of dropping out due to emotional stress. In some embodiments, the platforms, systems, media, and methods disclosed herein are configured for use by individual students or student populations. In such embodiments, the technology described herein is configured to connect users with relevant third party products and services from affiliate partners who lack the disclosed depth of AI insights. In further embodiments, the rewards and reward marketplaces described here include ties into college currency and currency systems.

### In-Between Visit Support and Remote Monitoring

In some embodiments, the platforms, systems, media, and methods disclosed herein are configured for use by individuals or populations receiving care from a healthcare or mental health provider to provide in-between visit support and to provide a mechanism for remote monitoring. In such embodiments, the technology described herein is configured for helping users with mental fitness, rather than providing medical treatment and/or diagnosis.

Well-being for Individuals with Trauma and PTSD

In some embodiments, the platforms, systems, media, and methods disclosed herein are configured for use by individuals or populations with trauma and conditions such as post-traumatic stress disorder (PTSD). In such embodiments, the technology described herein is configured for helping users with mental well-being, rather than providing medical treatment and/or diagnosis.

Well-being for Individuals in Recovery from Addiction

In some embodiments, the platforms, systems, media, and methods disclosed herein are configured for use by individuals, groups, or populations with addiction, substance use disorder, or those in recovery from such conditions. In such embodiments, the technology described herein is configured for helping users with mental well-being and mental fitness, rather than providing medical treatment and/or diagnosis.

### Well-being for Displaced Populations

In some embodiments, the platforms, systems, media, and methods disclosed herein are configured for use by individuals, groups, or populations displaced due to armed conflict, generalized violence, and/or human rights violations, such as refugees and refugee populations. In such embodiments, the technology described herein is configured for helping users with mental well-being. In further embodiments, the present subject matter is configured to preserve and protect the data privacy and security of individuals while applying the disclosed algorithms to identify and predict well-being insights at a group or population level.

### Survey Platform

Because existing survey tools lack the ability to efficiently and seamlessly collect audio files for survey purposes, in some embodiments, the platforms, systems, media, and methods disclosed herein are configured as a survey platform. In such embodiments, the survey experience comprises recording audio files in response to story prompts and the platform captures, transcribes, formats, and automatically analyzes those audio files, without introducing manual workflows.

In some embodiments, the platforms, systems, media, and methods disclosed herein are deployed as a web-based platform hosted with a unique URL. In some embodiments, the technology is configured as a well-being insights platform. In other embodiments, the technology is configured as a survey platform. In yet other embodiments, the technology is configured as both a well-being insights platform and as a survey platform.

In a particular embodiment, a survey platform deployed as a web-based platform hosted with a unique URL that connects to the back-end system described herein, which allows for automated analysis of survey data. For example, an exemplary user experience starts with a survey participant engaging with the platform, the participant views survey content, and records audio files in response to story prompts. The back-end system then runs automated processes to transform the audio files into usable survey data. Specifically, when an audio file is recorded the web-based survey platform, and transmitted to the back-end system, the system conducts automated processes comprising transcribing the audio, applying algorithm models, and storing the data outputs within the back-end system.

Many survey features are suitable, including but not limited to, single-line text input (i.e., name/date fields), paragraph/multi-line text input, rich text input (i.e., text formatting, titles, headings, etc.), multiple choice questions, sliding scale questions, survey routing logic, file/image upload, drop down options, radio buttons, and the like.

In the survey platform use case of the present technology, the data output of the system is focused on generating insights to support scientific research, and not as strongly focused on providing individual-level or population-level well-being insights to participants. In a particular embodiment, the survey platform is licensed to one or more third-party organizations for the purpose of conducting surveys and generating data insights from the surveys for the purpose of scientific research.

### Pod

In some embodiments, the platforms, systems, media, and methods disclosed herein are deployed and used, at least in part, in conjunction with a Pod or a Wisdom Pod. In some embodiments, the Wisdom Pod comprises a device or machine that a user optionally enters and occupies physically. In further embodiments, the Wisdom Pod is configured to allow a user to, enter the pod, receive a prompt to view content, and record stories. In some embodiments, the Pod includes computer hardware and software, as well as mechanical and/or robotic components, to conduct the functions described herein. In some embodiments, the hardware and software is self-sufficient and runs locally, without the need for communications to and from external networks and the like. In other embodiments, one or more functions, such as machine learning, generative AI art creation, and the like, are implemented and run externally and are accessed via communications with one or more external networks, such as a LAN or WAN, the Internet, a cloud computing platform, and/or a distributed computing platform.

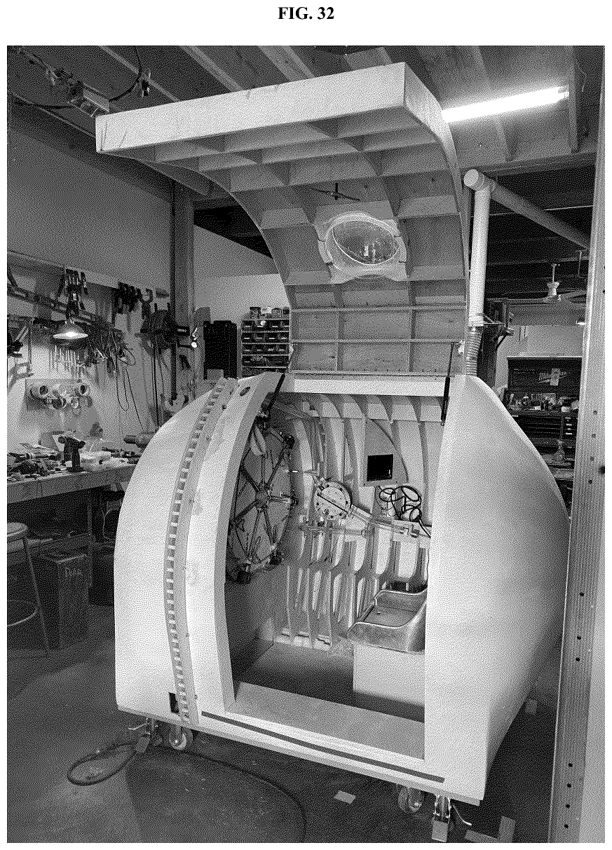

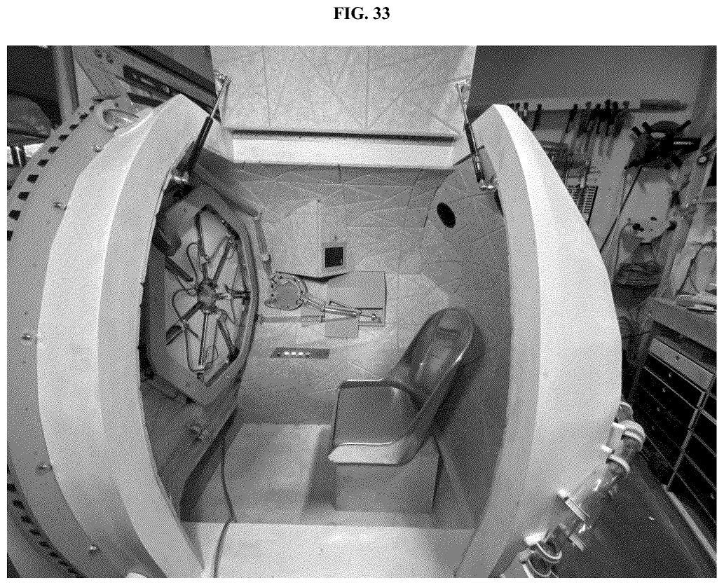

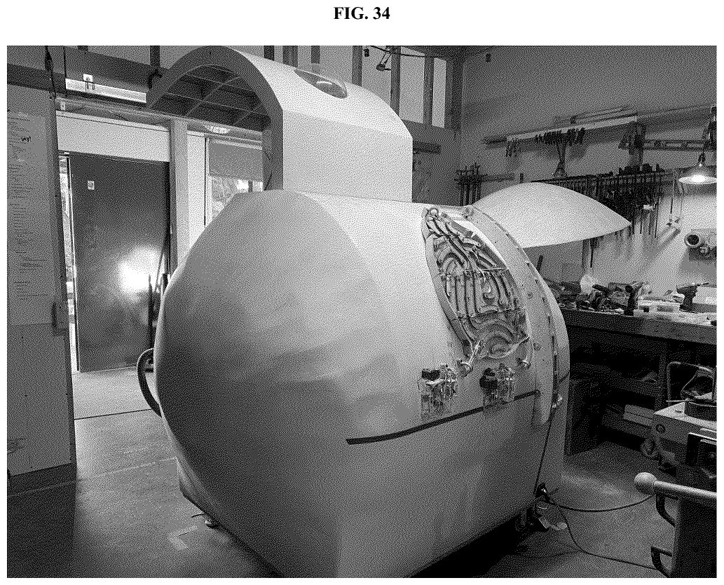

Referring to FIG. 32, in a particular non-limiting embodiment, a Wisdom Pod described herein is constructed of wood, metal, plastic, or any other safe and suitable materials, including combinations thereof. In this embodiment, the Wisdom Pod includes a body defining an interior and a first door allowing physical access to the interior. In this case, the first door opens vertically to expose the interior, which includes a seat, one or more screens, and user controls. Referring to FIG. 33, in a particular non-limiting embodiment, a Wisdom Pod described herein includes an insulated interior. Referring to FIG. 34, in a particular non-limiting embodiment, a Wisdom Pod described herein includes a first door and a second door. Referring to FIG. 35, in a particular non-limiting embodiment, a Wisdom Pod described herein includes a second door, opening vertically to provide access to computing equipment configured to allow the user to view content, record audio and/or video stories, and the like. Referring to FIG. 36, in a particular non-limiting embodiment, a Wisdom Pod described herein includes a user control panel comprising “launch” and “end” buttons.

In some embodiments, a user may use the Pod to share stories and help improve the user's well-being. The user may enter the Pod through an open hatch or door (see, e.g., FIGS. 32 and 33) and sit in the provided seat. Once the door is closed, the Pod may allow the user to fully immerse oneself into the experience. For example, the Pod may provide an environment visually and auditorily isolated from the outside. The Pod may provide a mechanism for the user to share their stories and provide a visual experience to sharing their stories. For example, the Pod may provide, via a display screen, prompts and choices for the user. The Pod may provide real-time feedback to the user via visual, auditory, olfactory, or tactile means. For example, the Pod can provide a visualization of the user's experiences and stories that are shared and help the user come to a better understanding of how to understand and view the experiences in the stories that are shared.

After experiencing the Pod, the user is able to feel rejuvenated and cathartic about the stories and be able to leave the experience with a new understanding of the stories that were shared. For example, if the user had a negative connotation towards the experience, the user may have a positive connotation towards the experience after being in the Pod.

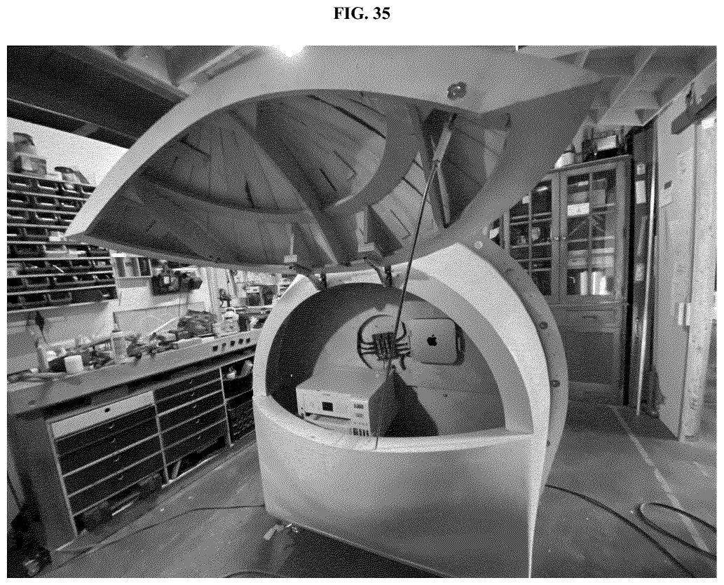

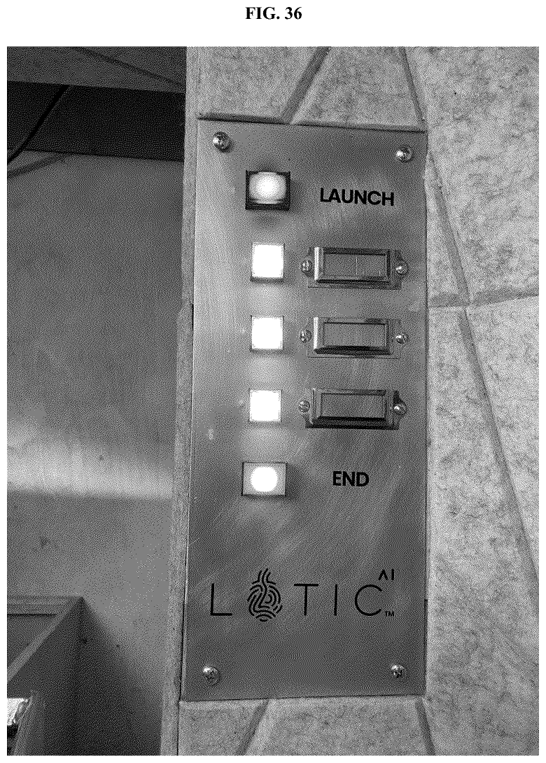

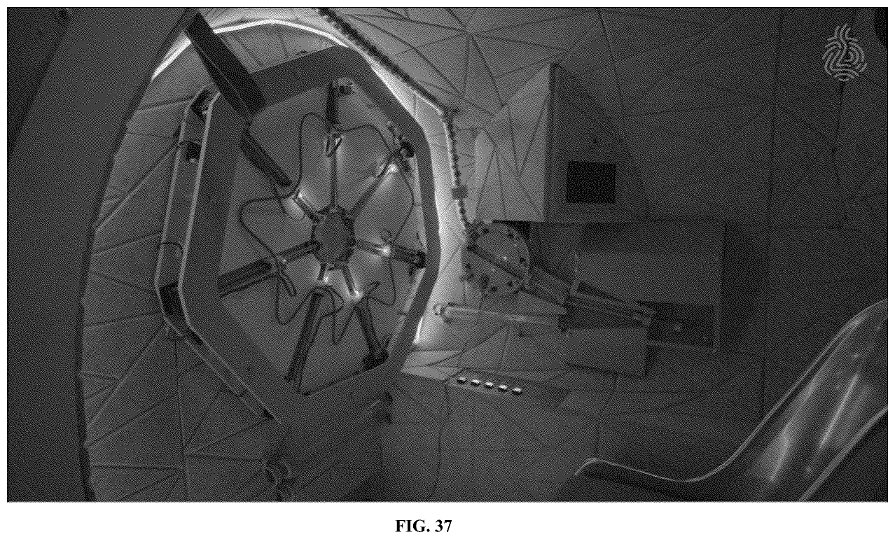

Once the user is sitting in the Pod, the door or hatch may close, and the user may sit facing a wheel that is configured to spin and light up in response to the user's stories (see FIG. 37). The wheel may have a heptagonal shape, but embodiments are not limited thereto, and the wheel may have any number of sides or not have a multilateral shape (e.g., circle, oval, etc.). In some embodiments, the wheel may include several light-emitting diodes (LEDs) that are configured to light up in one or more colors. The LEDs may be configured to move towards and away from a center of the wheel as the user is sharing. The LEDs may be configured to move along their own tracks individually or collectively. For example, the LEDs may flash to create a visual impression of moving lights, and the speed and distance of the motion may provide real-time feedback.

As the user is sitting in the Pod, the user may be asked via a prompt to share a story as discussed elsewhere herein. As the user is sharing the story, a microphone may pick up the sound, and one or more processors may process the story that is being shared in real-time or near real-time. The one or more processors may be encoded to identify recurring words, phrases, emotions, or concepts that and cause one or more of the LEDs to traverse along the rails based on the recurring words, phrases, emotions, or concepts. For example, if a red LED is encoded to respond to the term “father” or “dad,” the red LED may be moved towards the center of the wheel each time the term “father” or “dad” is used. In some embodiments, the red LED may also respond when the person referred to as “father” or “dad” is referenced using pronouns (e.g., “he” or “him”). In some embodiments, each of the LEDs may be configured to respond to more than one word, phrase, emotion, or concept. For example, the blue LED may be configured to respond to any negative emotion such as anger, sadness, fear, etc. Once the designated LED has reached the end of the track, the LED may be begin traversing the other direction. For example, if the LED has reached the end of the track close to the center of the wheel, the LED may begin traversing away from the center towards the edge. Then once the LED has reached a far end of the track, the LED may begin traversing towards the center again. Accordingly, the user may visualize how much or how little certain words, phrases, emotions, or concepts are dominant within the user's stories and therefore one's thoughts.

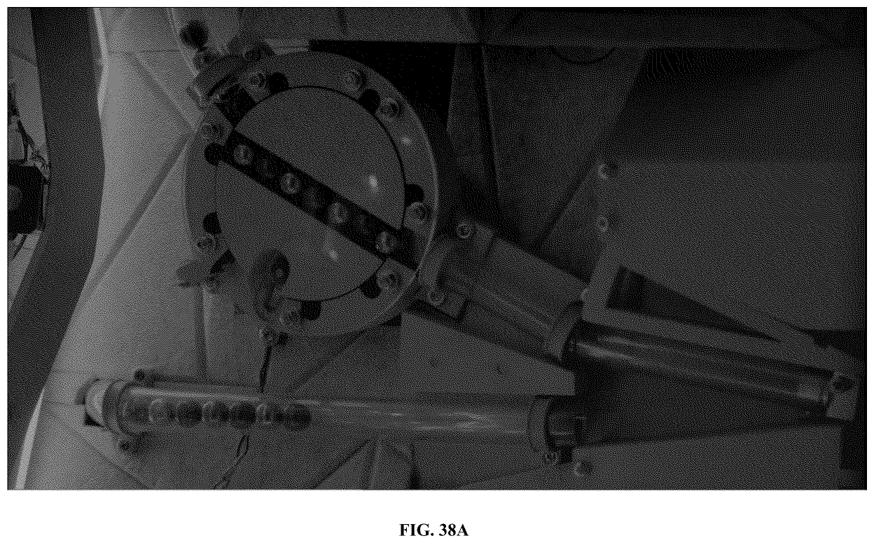

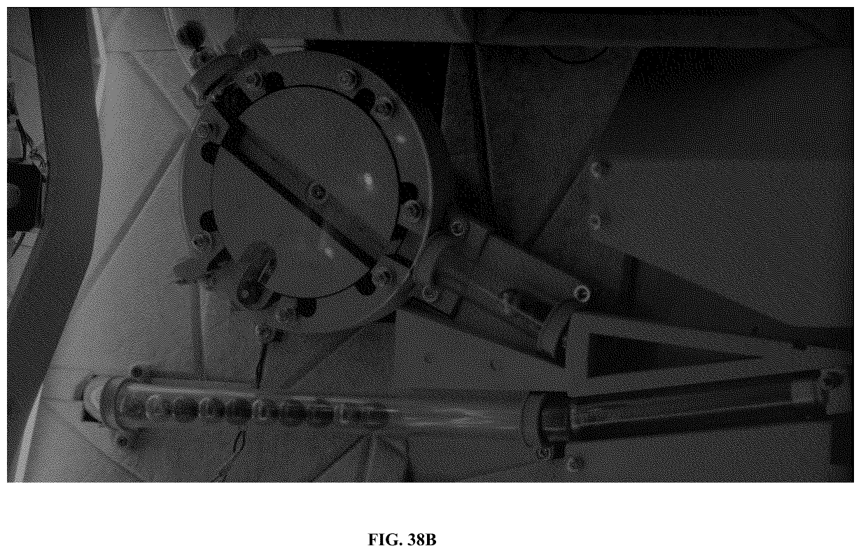

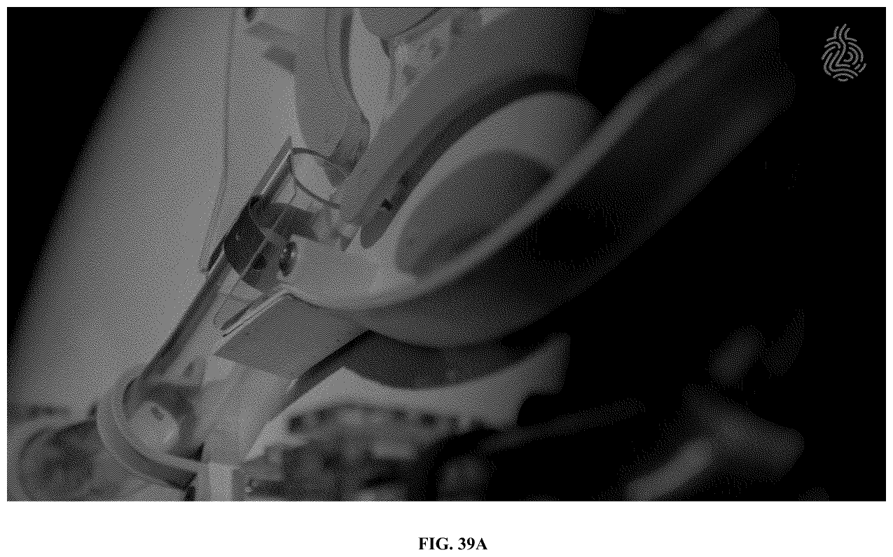

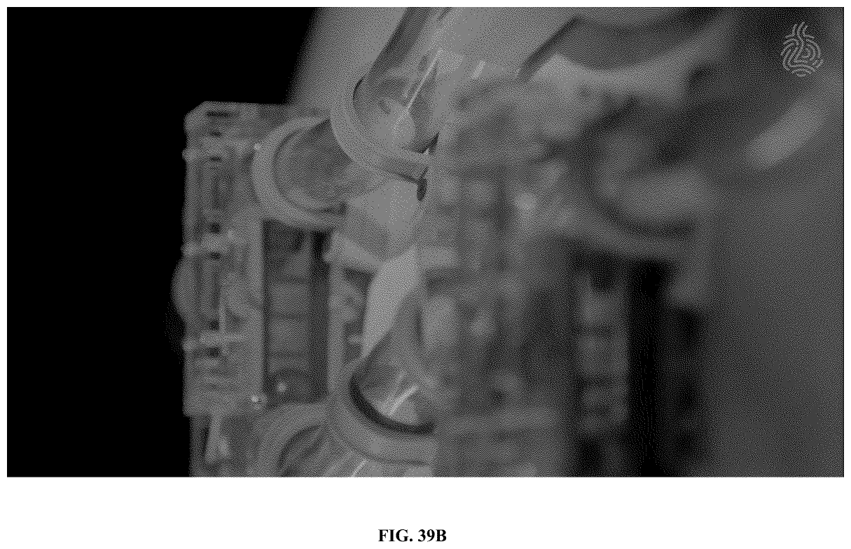

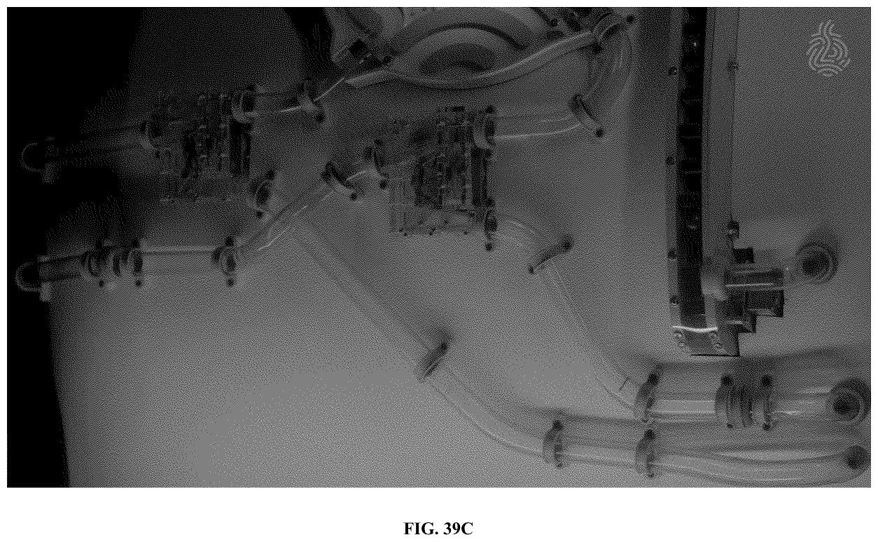

Referring to FIGS. 38A-39C, a plurality of beads (or balls) may traverse through a tubing system around the Pod as the story is being processed. The beads may also present a visual and physical instantiation of the data that was processed and/or generated by the user's story. For example, referring FIGS. 38A and 38B, the beads may be loaded in a rotating circular holder. The holder may have one entry point and one exit point. As the user is sharing the story, one or more words, phrases, emotions, or concepts that is described by the user may trigger the holder to rotate around an axis. Once the holder is in a certain position, the beads may be released through the exit point and into a tubing system. Referring to FIGS. 39A-39C, the beads may traverse through the tubing system that is set up in and around the Pod. The visual representation of the beads traversing the tubing system may help the user visualize and understand how different aspects of the user's experience as shared in the stories may help understand and internalize the user's experience. For example, the number of balls introduced into the tubing system may represent the amount of story content shared. In some embodiments, the balls traverse a track which starts inside the Pod and moves to the outside of the Pod to represent sharing. In further embodiments, the balls demonstrate to the user and observers that everything shared matters.

In some embodiments, a user's Wisdom Pod experience includes, or culminates in, generation of art as described herein. In further embodiments, the art is generated by one or more generative AI models to represent the experience, or one or more stories shared therein, and provide well-being-related insight(s) for the user, based on the experience and/or the stories shared.

### Generative Art

In some embodiments, the platforms, systems, media, and methods disclosed herein have features configured to generate art. In further embodiments, an artifact is generated based on one or more individual and/or group stories, or elements thereof, shared as described herein. Many types of art are optionally generated including, by way of non-limiting examples, one or more images, one or more videos, one or more audio clips, one or more music compositions, one or more interactive media, one or more augmented reality (AR) and/or virtual reality (VR) environments, and the like. In some embodiments, the art is generated as a well-being-related insight for the user. In various embodiments, the art is generated based at least on user context data and/or other data including, by way of non-limiting examples, themes, sentiment, intent, habits, patterns, beliefs, motivations, and/or recommended next actions. In various further embodiments, the color(s), shape(s), shade(s), contrast(s), form(s), speed(s) of animation, sound(s), and the like, are based on, or reflected in, one or more elements of the story, including user context data and/or other data. In some embodiments, the art is generated with the use of one or more generative AI models. The generative AI model may be configured to provide one or more outputs. For example, one or more outputs of the generative AI model may include a particular color(s), shape(s), size(s) (e.g., size of shape), position(s) (e.g., position of shape on the canvas), stroke thickness(s), hue(s), shade(s), contrast(s), form(s), speed(s) of animation, and/or sound(s), including tempo, melody, key, and the like, such that the one or more outputs may be used to in combination to generate the artifact. For example, the generative AI model may provide outputs of “blue” and “circle” to generate a blue circle on a canvas.

In some embodiments, one art piece is generated for a story shared. However, in other embodiments, multiple art pieces are generated for a story. In yet other embodiments, multiple stories are represented by a single art piece. By way of example, related stories or stories sharing a common characteristic, could contribute to, or be represented by, a common art piece providing one or more well-being-related insights for the user. By way of further example, stories shared over a period of time could contribute to, or be represented by, a common art piece providing one or more well-being-related insights for the user pertaining to the evolution of well-being.

In some embodiments, a set of custom or randomized assignments may be made to generate the artwork. By way of a non-limiting example, one or more aspects of the artwork may be generated based on, or influenced by, verb tense used, risk tolerance expressed, emotion expressed, sentiment expressed, pronouns used, themes addressed, people included, events included, and/or other characteristics of a user's story. In some embodiments, the technology described herein utilizes LIWC models. In some embodiments, the technology described herein utilizes one or more LLMs to identify, classify, and/or measure emotion and sentiment. By way of another non-limiting example, an assignment of “blue” for the word “work” or “job” may be made such that every time the user says the word “work” or “job,” the probability of the art generator service 3850 generating a blue shape is increased. As another example, an assignment “brush stroke” for the word “father” or “parent” may be made such that every time the user says one of these words, the art generator service 3850 generating a brush stroke on the canvas is increased. Accordingly, as the user shares their story, the artwork may be generated as the terms are discussed more by the user in their story. In some embodiments, the generative AI model may be configured to identify sentiment or emotions as discussed elsewhere herein. In some embodiments, the core backend 3815 may identify a variety of features from the story shared by the user, such as words, expressions, pauses, volume, intonation, pace of wording, etc. As discussed herein, the features may be processed and analyzed to determine insights.

In some embodiments, the platforms, systems, media, and methods disclosed herein features configured to generate art, e.g., an art generation service, which comprise multiple components. By way of example, in some embodiments, two components are used to operate the generative art features-a server and a client. In further embodiments, the server is responsible for receiving input data for generating the artwork, storing said data, storing the generated artwork, and notifying the user if they choose to receive a text message containing their generated artwork. In further embodiments, the client is responsible for retrieving the input data from the server, processing it in order to generate a video and an image from said data, requesting the user's phone number or email address, and uploading the artwork and the provided phone number or email address to the server.

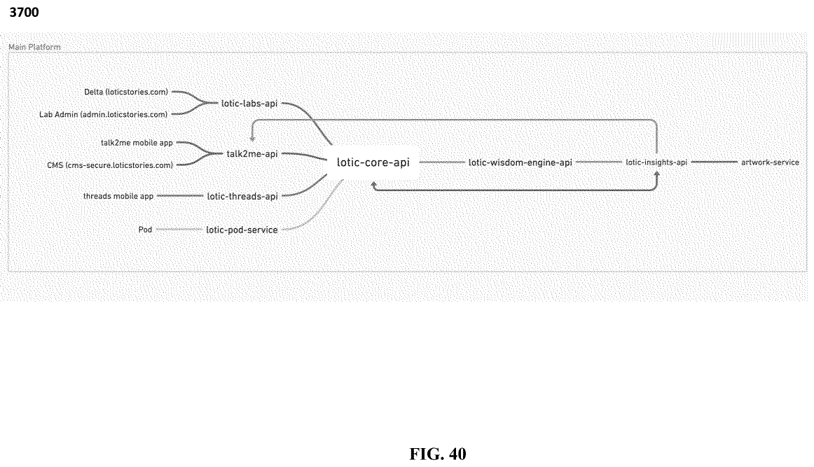

The art generation service described herein, in various embodiments, utilizes multiple components of the main platform. Referring to FIG. 40, in some embodiments, the main platform 3700 includes features feeding data into the Lotic Labs application, the talk2me application, the Lotic Threads application, and the Lotic Pod service. In the example of FIG. 40, these applications/services are in communication with the Lotic Core API, utilizing the Lotic wisdom engine API and the Lotic insights API to provide data to the artwork service described herein.

In some embodiments, when a user shares a story (whether in the Pod, via the Pendant, or elsewhere on the Lotic Platform), the input method (i.e., Pod, Pendant, mobile application, web site, etc.) receives the user's recording, processes the data, obfuscates the data, associates each obfuscated input with a color value, and sends that generated bundle of data to the server. In further embodiments, once the client retrieves the data, the client will begin to generate artwork based on that data. In still further embodiments, the user optionally enters their phone number or email address, and the generated artwork is sent to the server for storage. Finally, once the server receives the artwork and the phone number or email address, a text message or email is sent out to the user with a link to view their generated artwork.

### Process

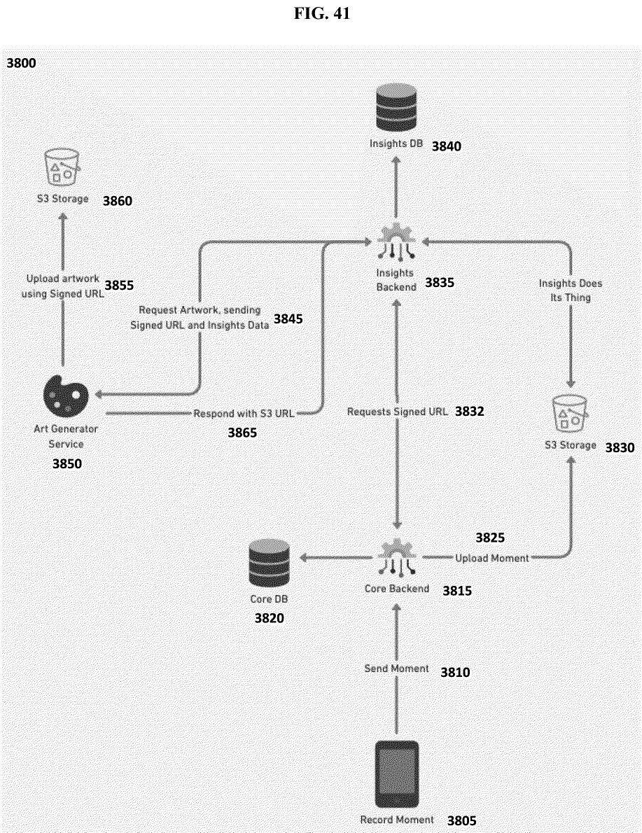

Referring to FIG. 41, in some embodiments, an architecture and process 3800 is provided wherein, when a user records a moment 3805 via an input method (i.e., Pod, Pendant, mobile application, web site, and the like), the input method receives the user's recording, processes the data, obfuscates the inputs, associates those inputs with a color value, and makes an HTTP request to the server to send the moment 3810 to the core backend 3815. The core backend 3815 stores the data in the core database 3820 and uploads the moment 3825 to AWS S3 storage 3830. In some embodiments, the color values are randomly selected. In other embodiments, the color values are based on the data.

In some embodiments, in order to allow the user to maintain agency over their personal data, the submission of this data creates a generic database entry and does not create a user profile. While not actively creating or uploading artwork, the client pings the server periodically (e.g., for example, every 1, 2, 3, 5, or 10 seconds, or more) to check if a new session is available. Once a new session is found, the client uses the data provided and begins generating the artwork.

Continuing to refer to FIG. 41, in some embodiments, the data is provided to the insights backend 3835 to generate insights for the user, as described herein, which are stored in the insights database 3840. Meanwhile, in some embodiments, the core backend 3815 also requests a signed URL 3832 from the insights backend 3835. The insights backend 3835 then requests generation of artwork 3845, sending the signed URL and the insights data to an art generator service 3850. The art generation service 3850 generates the artwork, uploads the artwork using the signed URL 3855 to AWS S3 storage 3860, and responds to the insights backend 3835 with a S3 URL 3865.

For example, in some embodiments, once artwork generation is complete, a prompt is shown on screen asking the user if they would like to enter their phone number or email. If the user chooses to enter their phone number or email, the generated screenshot, video, and thumbnails are uploaded to the server. Once the server receives the artwork from the client, it uploads those assets to an AWS S3 bucket. The server then uses text or email capabilities to send the message to the user containing the generated image of their artwork and a link to download the animated video. The client automatically resets and begins polling the server again for a new session.

### Exemplary Art

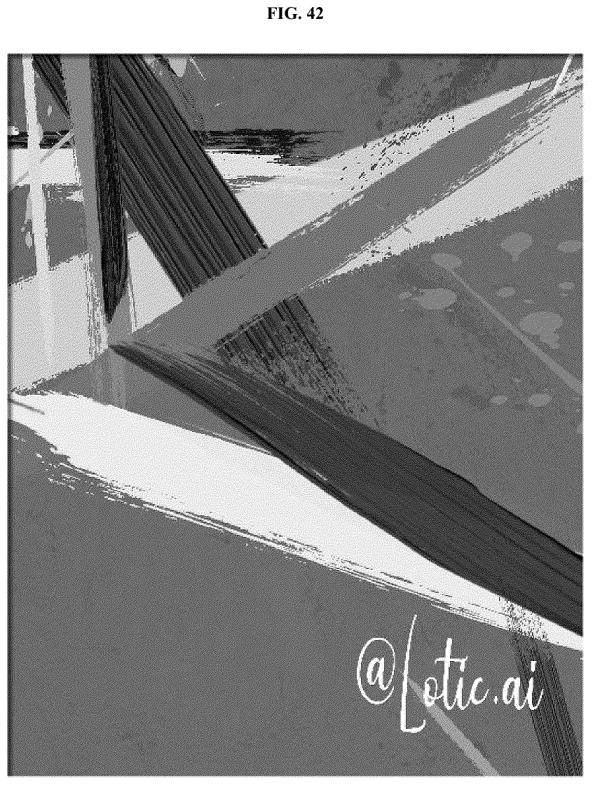

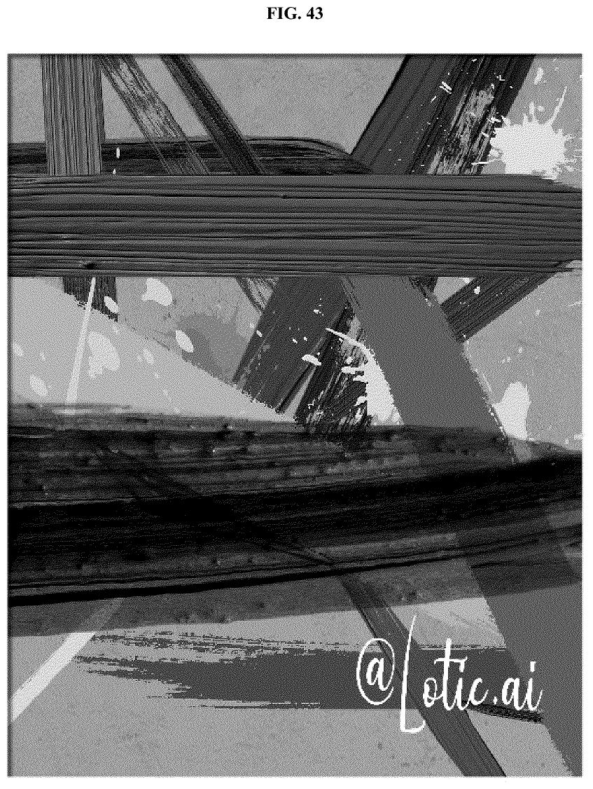

Referring to FIG. 42, a first exemplary still image is shown, which was generated using a generative AI model, based on a user's story. In this example, the color(s), shape(s), shade(s), contrast(s), form(s), and the like, are based on, or reflected in, one or more elements of the story. Referring to FIG. 43, a second exemplary still image is shown, which was generated using a generative AI model, based on a user's story.

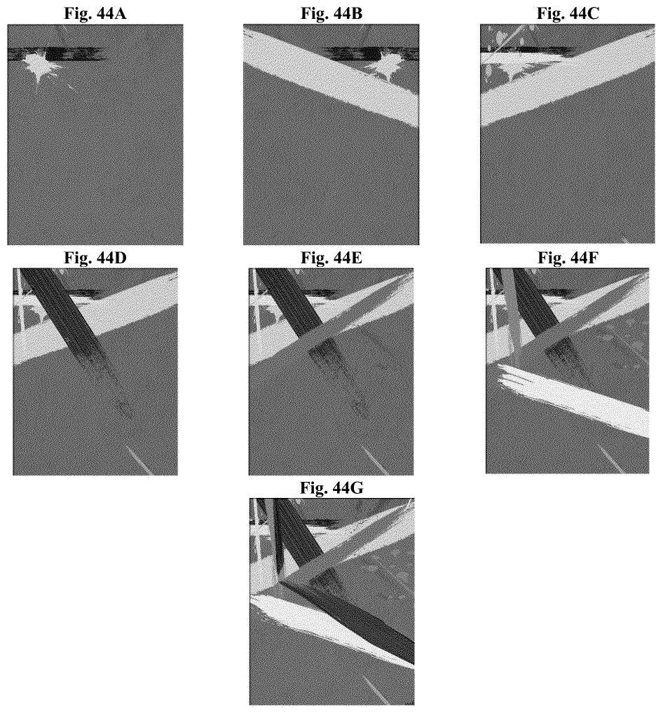

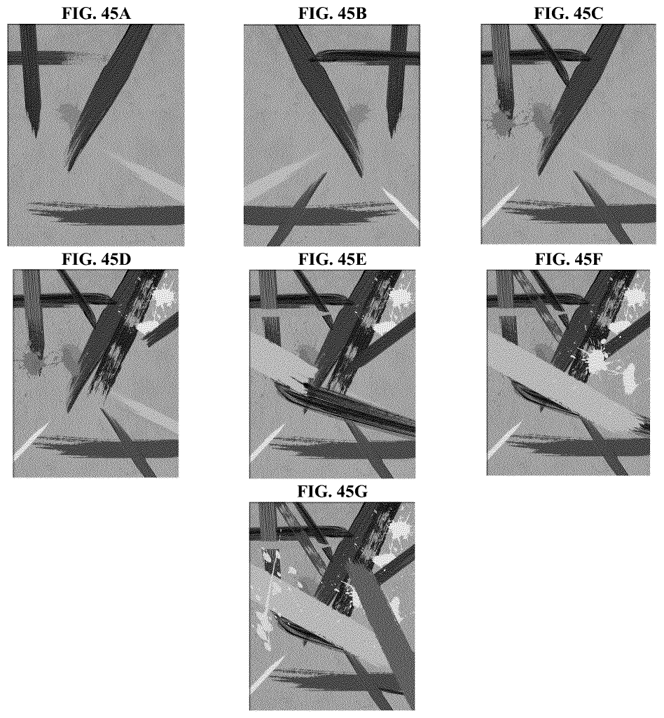

Referring to FIGS. 44A-44G, a first exemplary video is shown (as represented by a series of still images), which was generated using a generative AI model, based on a user's story. As shown in FIGS. 44A-44G, the video includes an animation (and optionally audio) of the artwork being created, wherein elements of the art are sequentially added, based on elements of the story. In this example, the color(s), shape(s), shade(s), contrast(s), form(s), speed(s) of animation, sound(s), and the like, are based on, or reflected in, one or more elements of the story, such as themes, sentiment, intent, habits, patterns, beliefs, and/or motivations. Referring to FIGS. 45A-45G, a second exemplary video is shown (as represented by a series of still images), which was generated using a generative AI model, based on a user's story.

### EEG Data

In some embodiments, an electroencephalogram (EEG) may be used to generate an artifact. For example, EEG may be used to generate music. Generally, as the user is sharing a story, the EEG may capture points of interest in the data to generate the music. The EEG data may be added to or supplement other collected data, such as data from the Pod or data collected via the wearable device (e.g., pendant). The collected data may be used to generate the music.

Various methods to obtain and/or capture EEG data are suitable. In some embodiments, a user wears an EEG capture headset (such as, by way of non-limiting example, a Muse headset) while engaging with the system described herein. In other embodiments, EEG data is obtained from a third-party. In a particular embodiment, EEG data associated with the frontal lobe of a user is advantageous for generating artwork and representing well-being-related insights.

In some embodiments, as the user is speaking, the EEG data may be turned into music. For example, the EEG data, which is a collection of electrical activity of the brain, may identify various points of interest that could be used to generate the music. For example, the voltage fluctuations in the EEG data may be indicative of the user's feelings or emotions towards what the user is speaking about. In some embodiments, the user's story may be paired to the EEG data such that what word was used at the points of interest may be identified. The EEG data can be processed in real-time or near real-time and used to generate the music.

In some embodiments, once the EEG data is processed, a generative AI model may be used to generate the music. In some embodiments, each output of the model may be assigned to provide one in a range of instruments, notes, meter, beats, rhythms, keys, chords, and other components of a song. In some embodiments, the AI model may be trained on one or more rules about music composition. For example, a rule may indicate that the song should not have any consecutive octaves or consecutive fifths. In some embodiments, the AI model may not be trained on any rules. In some embodiments, the course or arc of a story is represented by the characteristics of the generated music.

In some embodiments, the generated music can be converted into sheet music and/or digital files that can be used to play in audio players. In some embodiments, the user may generate multiple songs and compiled together into an album.

In some embodiments, EEG data is not needed to generate music. For example, music may be generated based on other data points such as pitch, tone, emotional sentiment, and other features of a user sharing their story out loud, absent any EEG data. The user may share their story out loud, and the system may capture the story, apply one or more models to assign different sounds (musical notes, different instruments, etc.) based on the data points from the user's story (pitch, tone, sentiment, etc.), and generate a piece of music for the user.

### Third Exemplary Embodiment

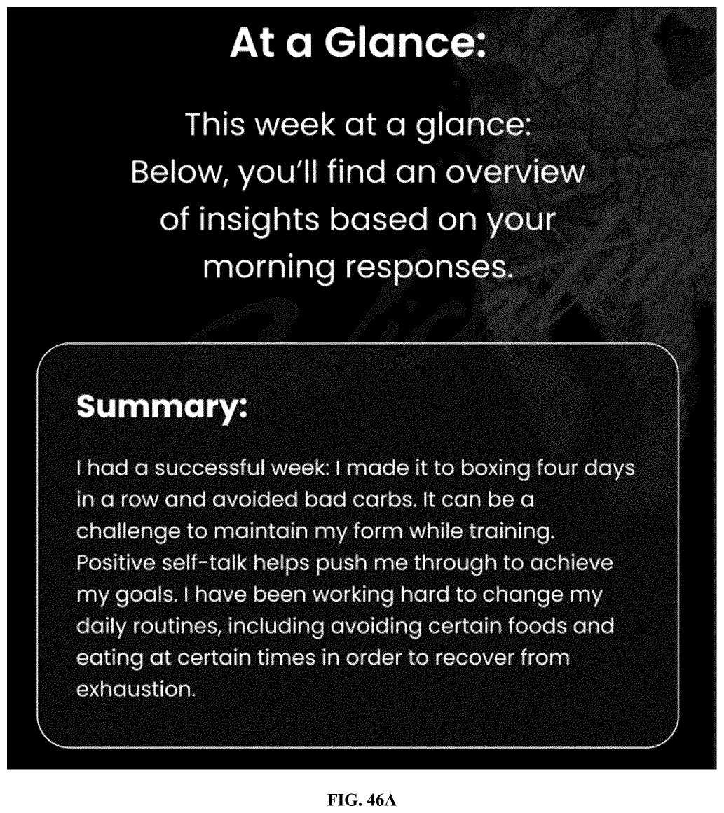

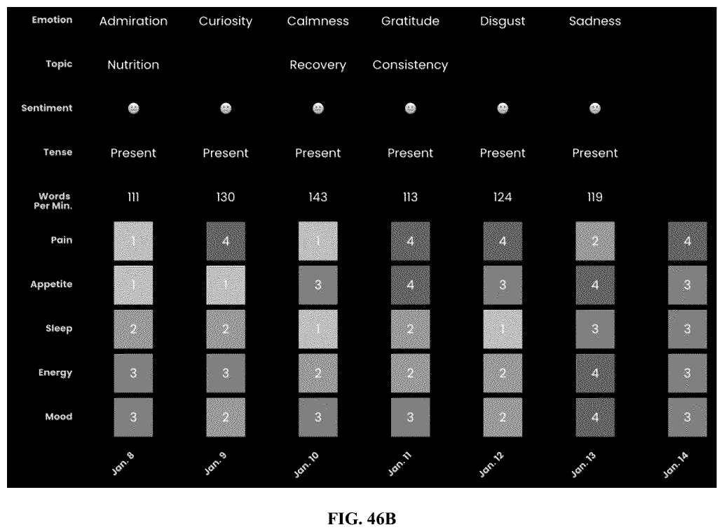

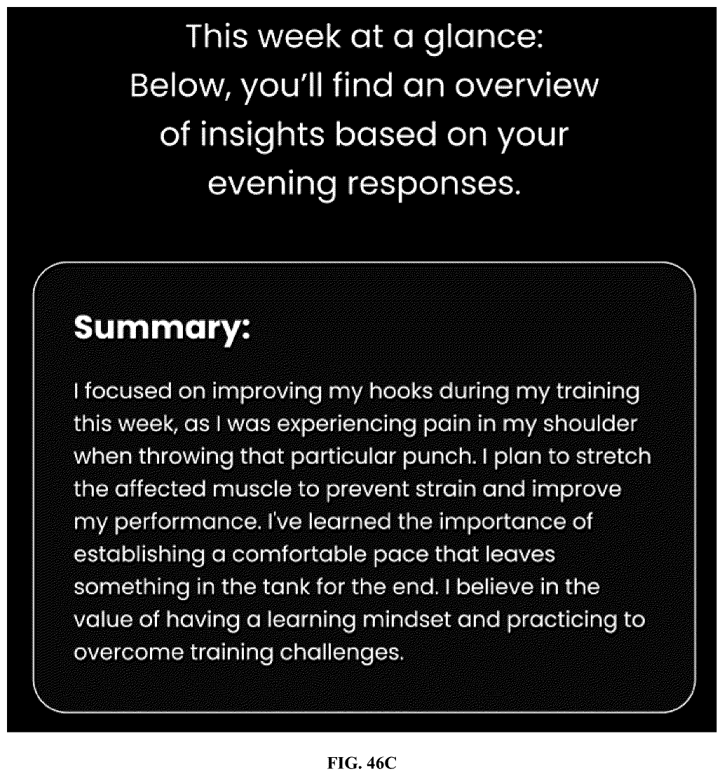

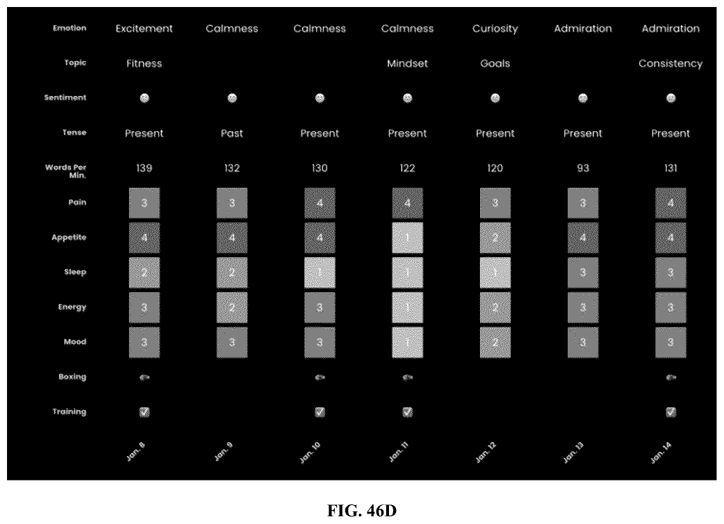

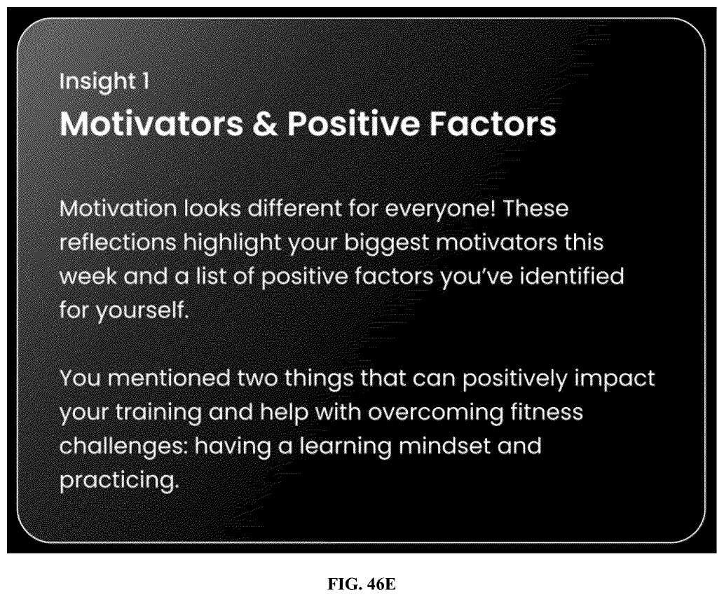

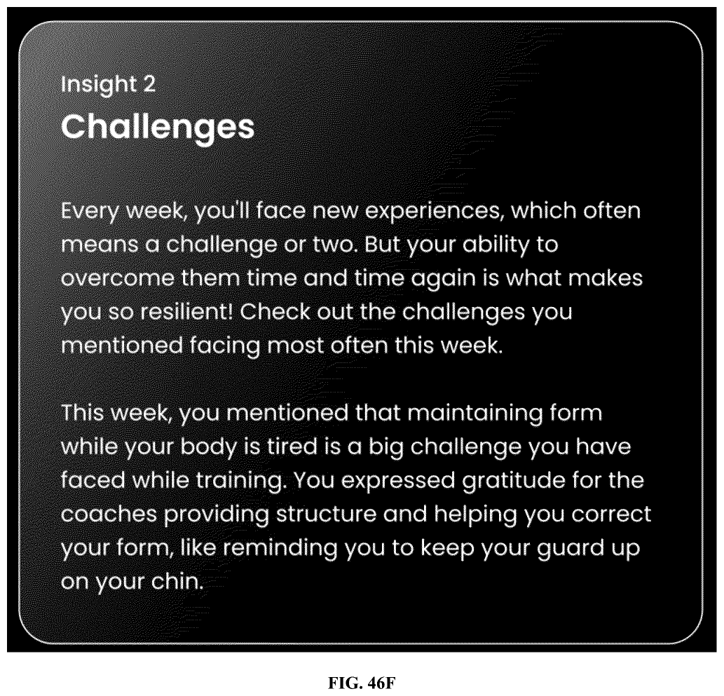

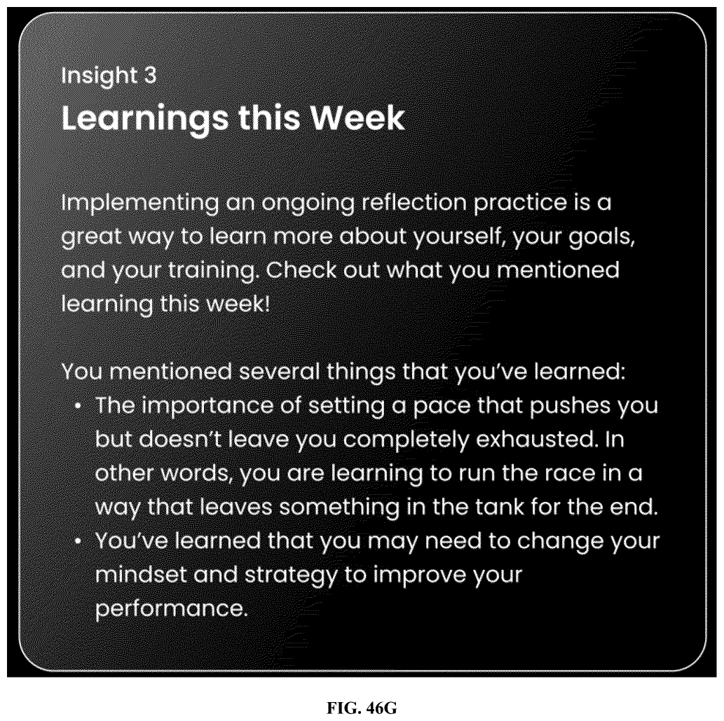

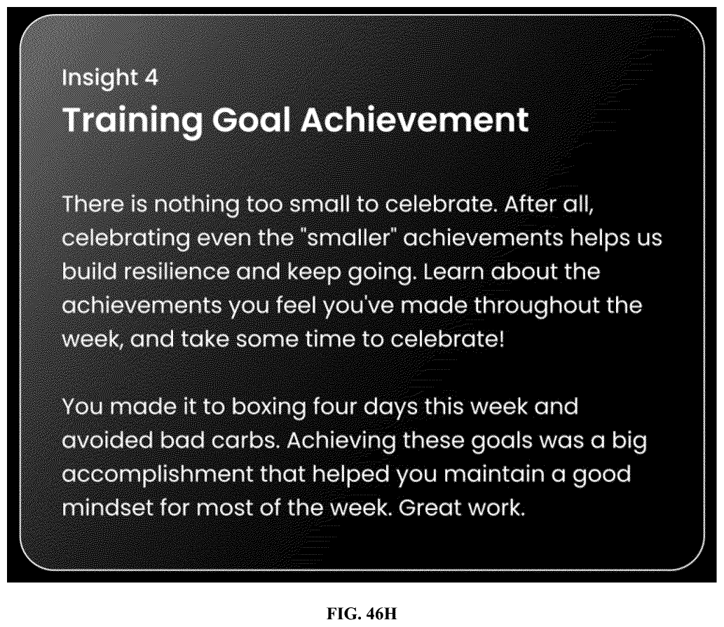

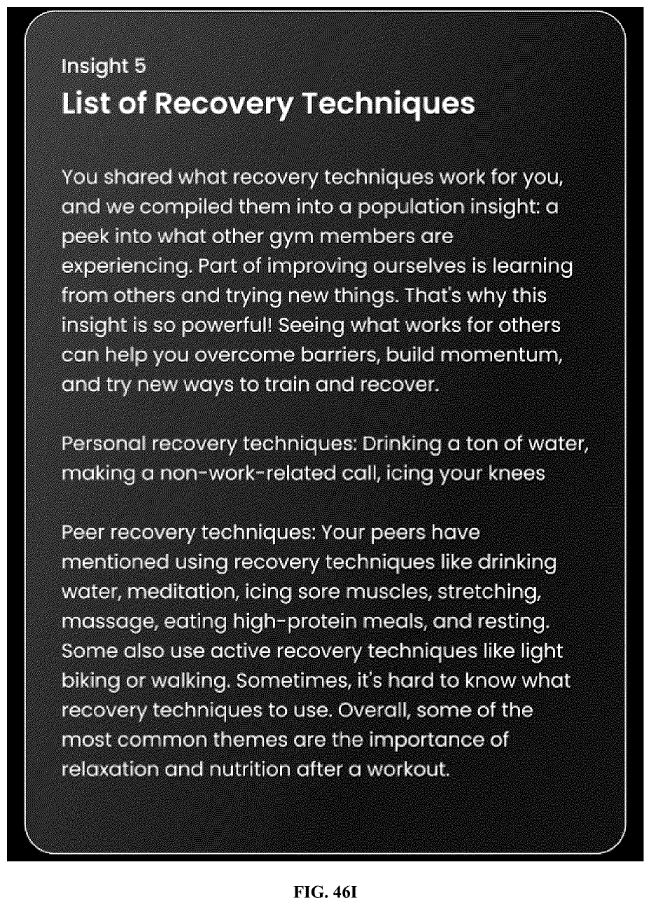

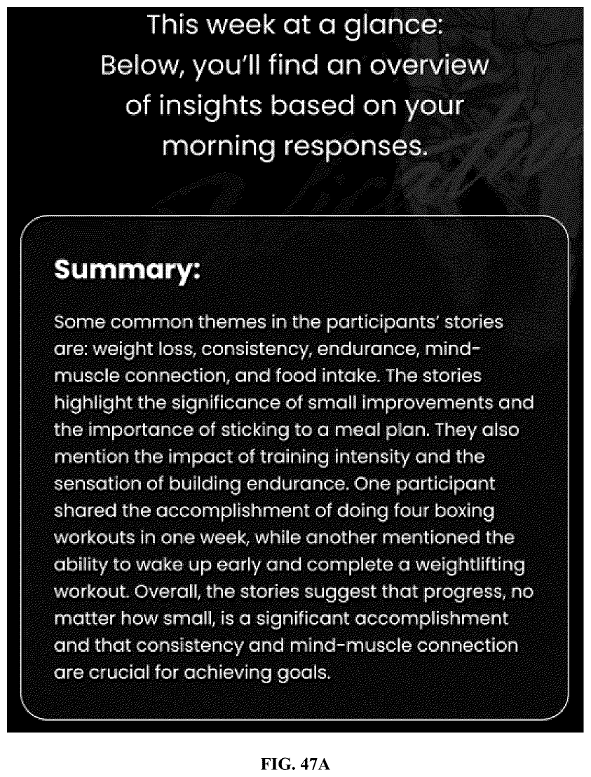

FIGS. 46A-46I show a non-limiting example of collecting stories and deriving insights that are for an individual. In this example, a boxing gym is interested in improving the well-being of its members. The improved well-being can also improve the individual's performance in the boxing gym and help the individuals succeed in achieving goals, both within and outside the boxing gym.

The boxing gym may announce the well-being improvement program to its members and to sign up using, for example, a phone number. The user may provide his phone number in order to sign up for the program. The user's phone number can be used to generate a unique identification (ID) number which is used to identify the user's progress and store any insights derived from the user's input. Once the user provides his phone number, the system may encrypt any association between the user's phone number and the user's identity. For example, if the user does not provide any information about himself, including name or address, or any personal details about himself such as height or weight, the system can create the unique ID for the user and use the unique ID to store any information about the user. The unique ID may be stored in a data table so that the user's responses (e.g., stories) can be stored in the data table. Furthermore, the system can communicate with the user via the phone number that he provided, without the system knowing the user's identity. In order to protect the user's privacy, the system may permanently and/or irrevocably disassociate the user's identity from the user's phone number. At some point after the program has begun or after it has ended, the user may wish to provide his identifying information.

After the program has begun, the user may be asked to provide a story based on a prompt as discussed herein. The user may be asked to provide the input twice a day, but embodiments are not limited thereto, and the user may provide more or fewer stories throughout the week. In some embodiments, the prompt may ask a variety of prompts, such as “How do you feel this morning?” or “How did you sleep last night?” The user's responses may be recorded as moments or stories and provided to the insights backend to generate the insights. At the conclusion of the week or some regular basis, the insights backend may provide a summary of the user's insights for the mornings of that week, as shown in FIG. 46A. The user's summary may be provided in the voice of the first person so that the insights may resonate better with the user's experience when reading the summary.

The user's responses for the week's mornings may be recorded and saved in the core database. In some embodiments, the user's stories may be analyzed and provided in a chart, as shown in FIG. 46B. The chart may show, for example, the user's emotion, topic (of focus for the day), sentiment (in an emoticon or word or different format), tense used in providing the story, and the number of words per minute in the story. In some embodiments, the chart may also provide the user's pain level, appetite level, sleep level, energy level, and mood level on a scale of 1 through 5 that is color-coded. In some embodiments, the 1 may indicate the most negative or lowest level and 5 may indicate the most positive or highest level. In some embodiments, the color-coding of a lighter shade may be tied to the lower levels and a darker shade may be tied to the higher levels. However, embodiments are not limited thereto, and the scale can have any numerical range or color-coding.

Similar responses may be recorded, based on the same or different prompts, to record and display the insights of the user's evenings for that week, as shown in FIGS. 46C and 46D. In some embodiments, the prompts for the evenings may include some questions such as “Did you go to the gym today? If so, how was the training?”

The insights backend may be tailored to draw specific types of insights. For example, the insights backend may draw motivators and positive factors (FIG. 46E), challenges (FIG. 46F), learnings of this week (FIG. 46G), training goal achievement (FIG. 46H), and list of recovery techniques (FIG. 46I). These insights may be provided to the user at the end of each week and/or at the end of the program so that the user can track their progress. For example, the user may be given a prompt such as “Describe how you balance training with other life responsibilities. What challenges do you face in maintaining this balance?” The user may provide a response such as: “There's definitely a challenge in balancing other things that are going on in my life with this because it just takes so much time to get to and from the studio. So that's a constant thread of mine. The to and from, time driving there, time driving back. Um, I mean, I think I'm in a better mood when I do it. I'm definitely in a worse mood when I don't. So that's one of the things that, uh, where I'm having to balance and again, balancing it against work, balancing it against family. Um, all those sorts of things and that's, that's been a challenge for sure. And then I guess the other thing where I just need to balance is my body. My body really is not ready to, uh, go at it seven days a week, twice a day. Like it's just not ready to do that. So anyway, those are, those are some things. But, um, yeah, it's, it's difficult to balance but definitely you can find a sweet spot.” Such a response may generate insights and the output shown in FIGS. 46A, 46C, and 46E-46I.

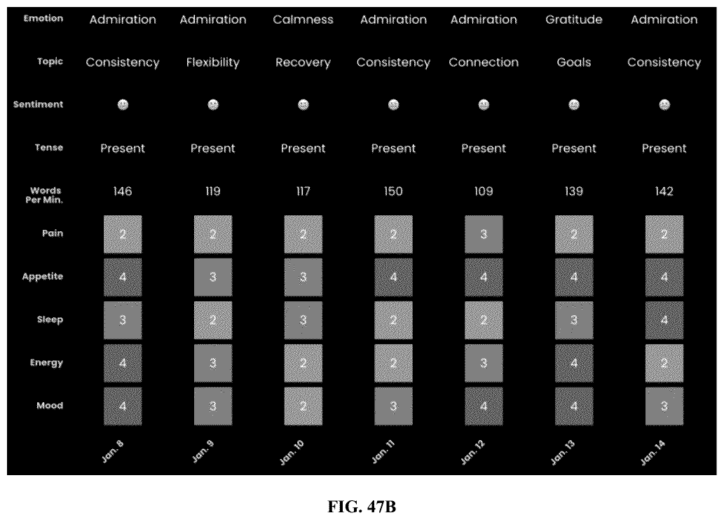

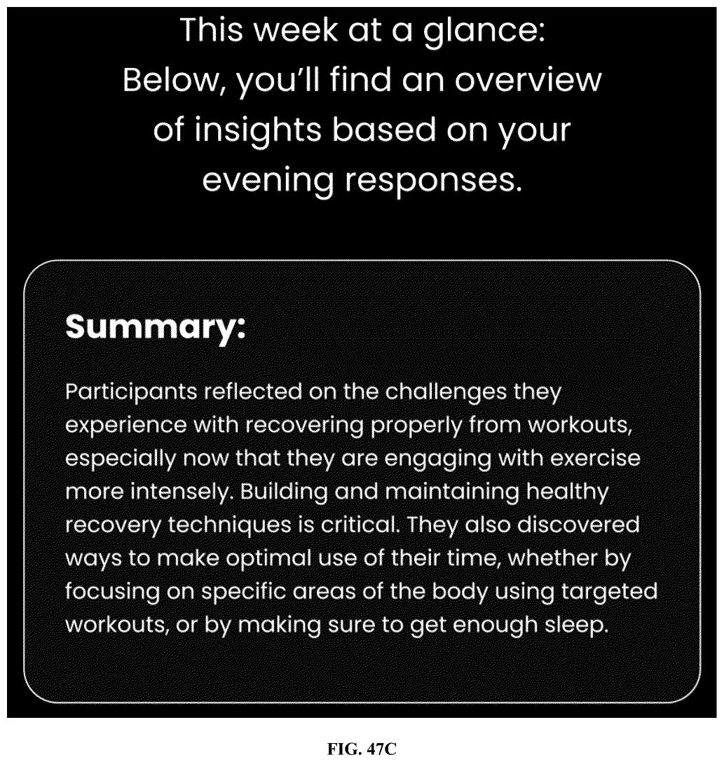

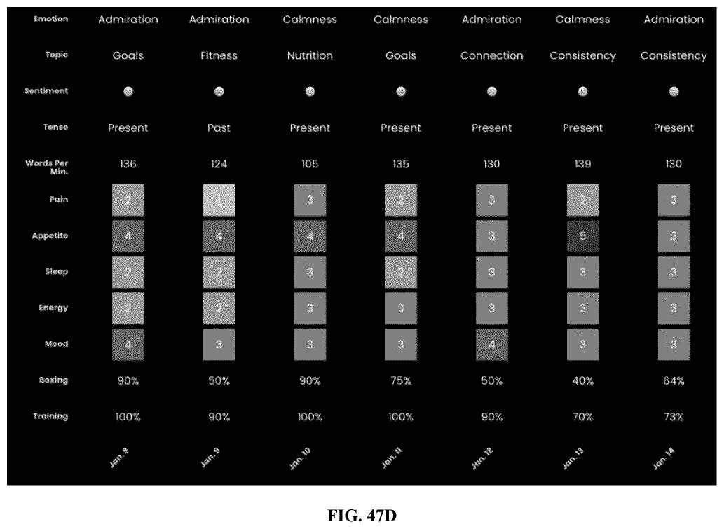

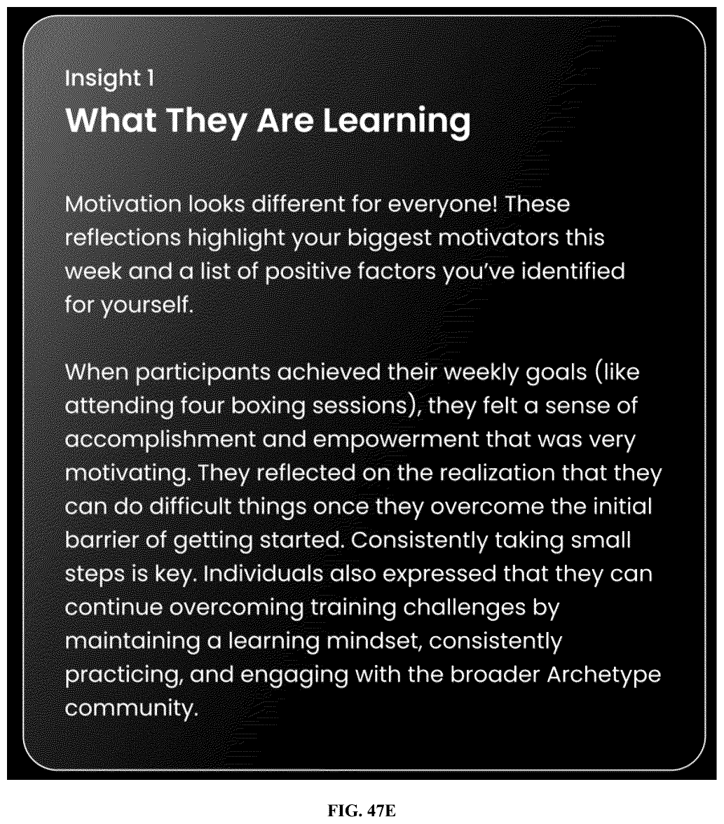

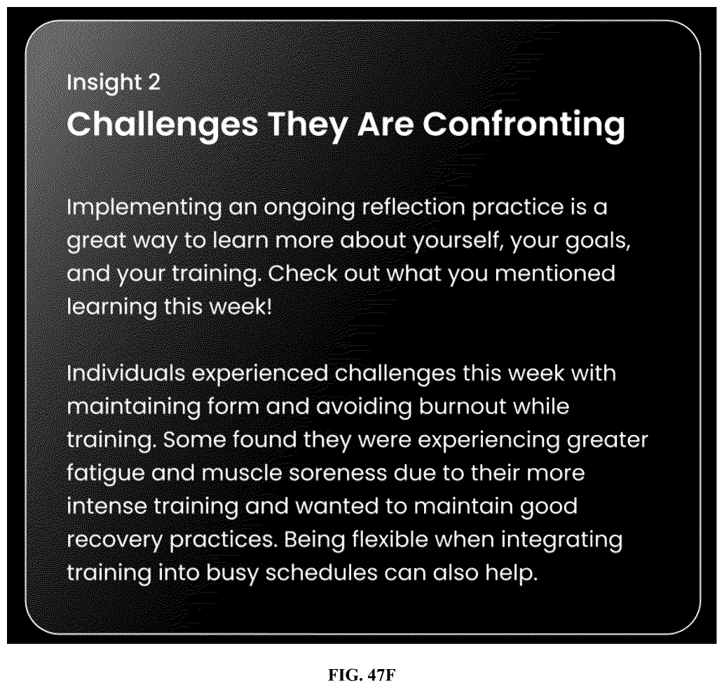

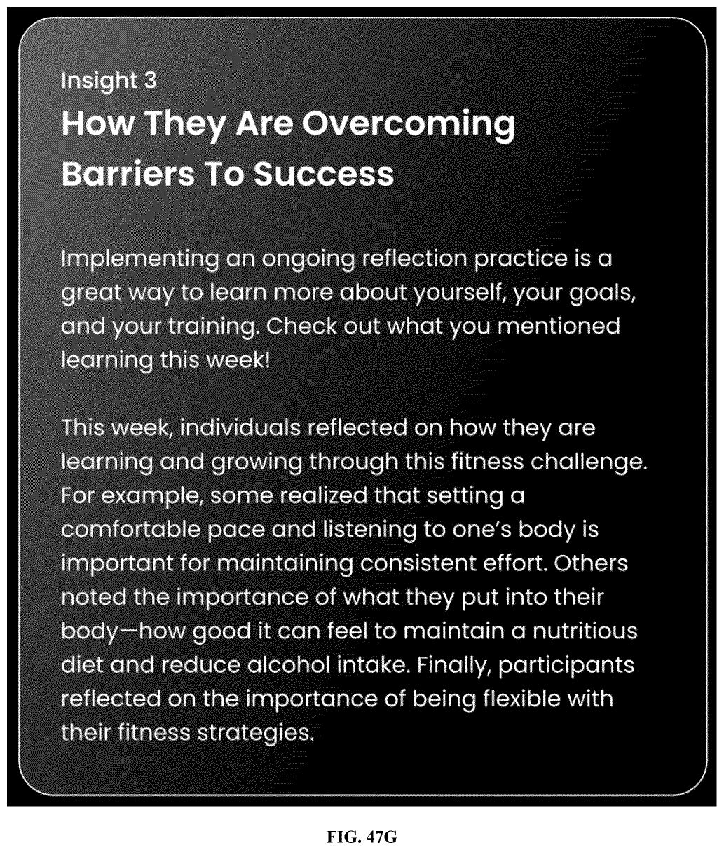

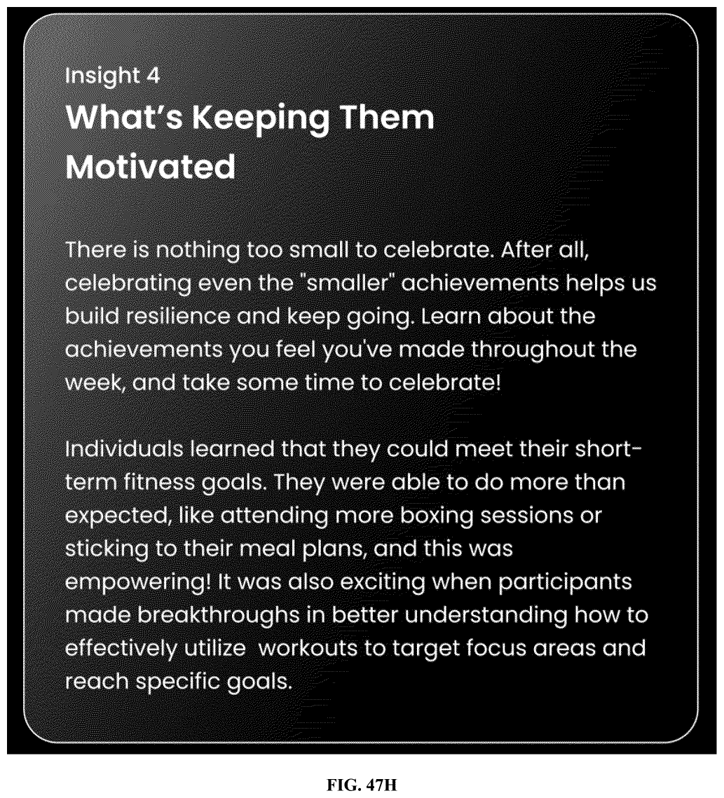

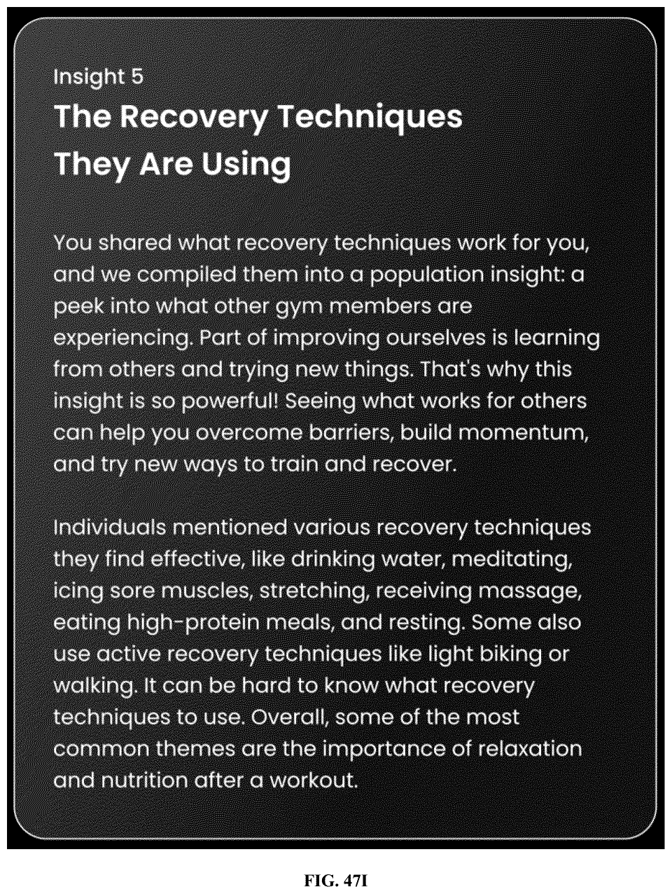

FIGS. 47A-47I show a non-limiting example of collecting stories and deriving insights for a group of individuals. Referring to the boxing gym described above, the responses and insights for all of the participants may be collected and compiled for the boxing gym director to review. For example, the morning responses for the participants may be compiled into one summary for the participants as shown in FIG. 47A, and the corresponding compiled chart may be shown in FIG. 47B. Similar compiled summary and chart may be displayed as shown in FIGS. 47C and 47D, respectively. Then, the director may be shown tailored insights directed to what the participants are learning (FIG. 47E), challenges the participants are confronting (FIG. 47F), how the participants are overcoming barriers to success (FIG. 47G), what's keeping the participants motivated (FIG. 47H), and the recovery techniques the participants are using (FIG. 47I). Then, the director may be able to gain insights regarding the program's participants on a weekly basis for example and make any adjustments to the program as desired in order to improve the participants' well-being.

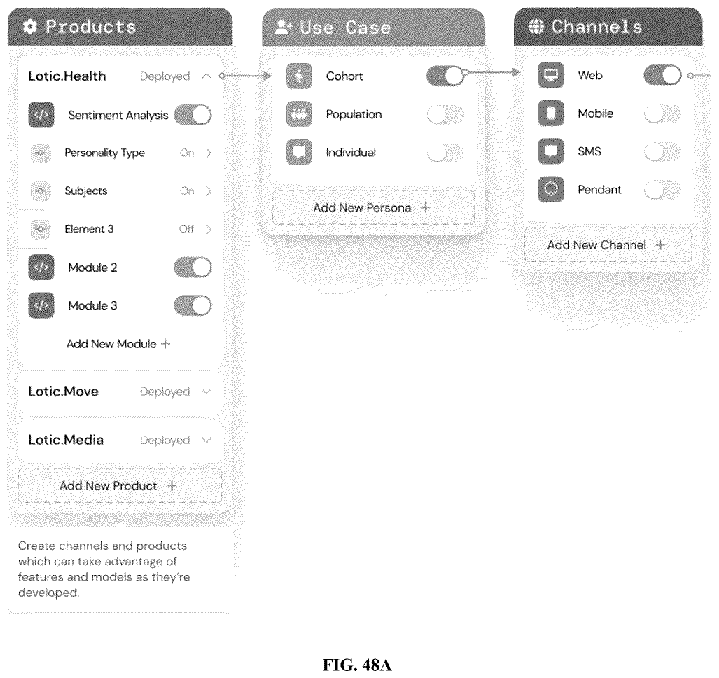

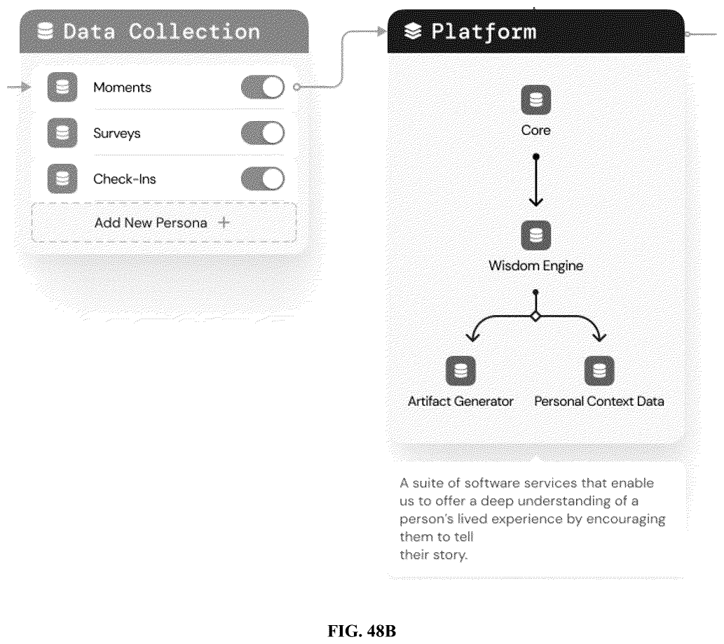

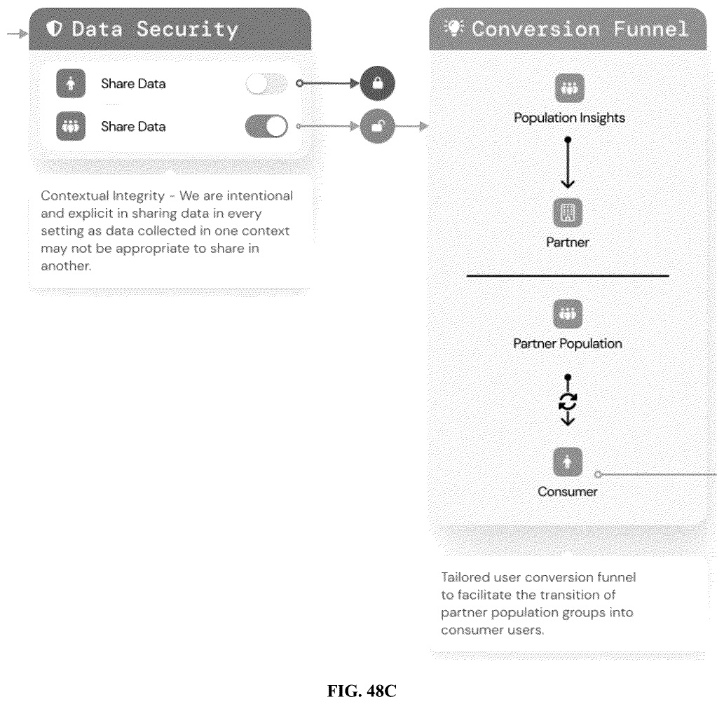

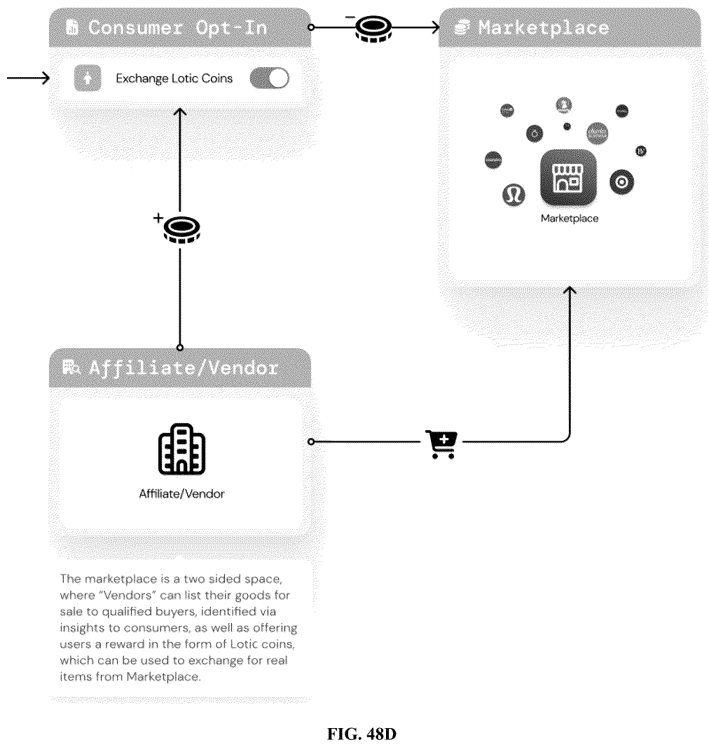

FIGS. 48A-48D show a non-limiting example of a general workflow for implementing the described technology. FIGS. 48A-48D are intended to be shown as an example, and one or more options in the figures may be modified or removed, and one or more additional options may be added. Referring to FIG. 48A, the administrator may select one or more products for deployment. In some embodiments, the Lotic. Health product may include tracking the well-being of a community or population of people using any of the technologies disclosed herein. In some embodiments, the Lotic. Move product may include a time-limited project, such as the boxing gym example described with respect to FIGS. 46A-47I. Within each product, one or more modules such as Sentiment Analysis may be deployed. For Sentiment Analysis, different types of insights may be derived and collected, such as the personality types (e.g., different insights with respect to sentiment, intent, patterns, beliefs, motivations, etc.), subjects (what participants are talking about), and others.

In some embodiments, one or more use cases may be selected. The cohort use case may include a group of people who have a common experience, such as a war, participating in a well-being improvement project at a boxing gym, etc., as described elsewhere herein. The population use case may include all people in the system or those who have a common characteristic such as being employees of the same company, etc. The individual use case may include using the product for selected individual persons.

In some embodiments, one or more channel(s) may be selected for deployment. For example, a web interface may be selected if the user wants to use a website or if the administrator desires to enable the web interface. Other channels include mobile (e.g., using a mobile application or mobile version of the website), text messaging via SMS whereby the user may provide stories via recordings or transcribed text, and the pendant for recording stories. In some embodiments, the administrator may select more than one channel for deployment (e.g., select all of web, mobile, SMS, and pendant or a subset thereof).

Referring to FIG. 48B, different types of data collection may also be selected. Data collection may be performed via moments where the user verbally shares a story in response to a prompt as described elsewhere herein, surveys whereby the user fills out answers to questions, and/or check-ins whereby the user shares, for example, a scalar value between 1 through 5 in response to a question such as “how stressed are you feeling today?” In some embodiments, multiple forms of data collection may be performed. By selecting the products, use case, channels, and forms of data collection, the basic components of the platform may be deployed. As discussed elsewhere herein, the core backend may be used to provide information such as stories which is provided to the wisdom engine that derives insights. The data provided to the wisdom engine may also be used to generate artifacts using the artifact generator (e.g., to generate the artwork or music) or personal context data such as personal insights.

Referring to FIG. 48C, additional options may be selected depending on the use case. In some embodiments, the data security setting may indicate what kind of data may be shared. For example, a setting for sharing the data of individual users may be toggled off so that an individual participant's information is protected and not shared with third parties. In some embodiments, data for a group of people such as a cohort or population may be shared when toggled on such that the group's general data, sentiment, and insights may be shared. In some embodiments, the individual participant may choose to opt-in for individual data sharing.

Depending on the data that is shared, the insights may be used to generate partners. For example, if a boxing gym signed up a group of participants to use the described technology to generate insights, and if the participant felt that the insights were beneficial and positively contributed to the participant's well-being, the participant may desire to continue using the platform. Such a participant may be categorized as a partner. One or more partners or a group of partners may become consumers as well.

Referring to FIG. 48D, a consumer may have various ways to participate in the marketplace as described elsewhere herein. In some embodiments, if a consumer desires, the consumer may participate in the usage of Lotic coins or tokens that can be exchanged for products and/or experiences. For example, if a participant successfully completes boxing gym challenge by recording their stories during the duration of the challenge, the participant may choose to continue using the product(s). Then, the participant, who is now a consumer, may use the coins in the marketplace to obtain products and/or experiences. The products and experiences offered to the consumer may be tailored based on the insights that are derived from the participant's stories, surveys, and check-ins. Affiliates and/or vendors may choose to participate in the marketplace by offering the products and experiences.

While certain embodiments of the present subject matter have been shown and described herein, it will be obvious to those skilled in the art that such embodiments are provided by way of example only. Numerous variations, changes, and substitutions will now occur to those skilled in the art without departing from the present subject matter. It should be understood that various alternatives to the embodiments of the present subject matter described herein may be employed in practicing the present subject matter.

1. A computer-implemented method comprising:
a) receiving media comprising an unstructured user-generated narrative;
b) applying an algorithm to the unstructured user-generated narrative to extract semi-structured user context data;
c) applying a first machine learning model to classify one or more of sentiment, intent, habits, patterns, beliefs, and motivations from the user context data, wherein the first machine learning model comprises an unsupervised machine learning model;
d) applying a second machine learning model to identify a recommended next action from the user context data, wherein the second machine learning model comprises a supervised machine learning model;
e) generating a well-being-related insight for the user based on the user context data, and one or more of the sentiment, intent, habits, patterns, beliefs, motivations, and the recommended next actions;
f) generating an interactive representation, wherein the interactive representation is generated in real-time from one of the user-generated narrative and well-being-related insights, and wherein the interactive representation comprises a virtual reality environment via visual, auditory, and tactile output devices, a music composition, or an image; and
g) fingerprinting the user-generated narrative, wherein the fingerprint authenticates the identity of the user.

2. The method of claim 1, wherein the media comprises audio.

3. The method of claim 1, wherein the media comprises video.

4. The method of claim 1, further comprising providing a prompt to guide the user in creating the user-generated narrative.

5. The method of claim 4, wherein the prompt is generated by a third machine learning model.

6. The method of claim 1, further comprising applying a quality metric to the media.

7. The method of claim 1, further comprising transcoding the user-generated narrative.

8. The method of claim 1, further comprising transcribing the user-generated narrative.

9. The method of claim 1, wherein the user context data is selected from a group consisting of: a personality metric, a personal theme, a speech pattern, a stress metric, a user motivation, and an emotional well-being metric.

10. The method of claim 1, further comprising applying a third machine learning model to generate media comprising a summary of the unstructured user-generated narrative.

11. The method of claim 1, wherein the well-being-related insights comprise a prediction.

12. The method of claim 1, wherein the well-being-related insights comprise detection of suicidal ideation.

13. The method of claim 1, wherein the well-being-related insights comprise AI generated artwork.

14. The method of claim 1, wherein the user context data comprises sensor data.

15. The method of claim 14, further comprising a wearable sensor and wherein the sensor data comprises wearable sensor data.

16. The method of claim 15, wherein the wearable sensor data is selected from the group consisting of: heart rate, heart rate variability, activity data, and sleep data.

17. The method of claim 15, further comprising providing general wellness curricula for the user based on the well-being-related insights.

18. The method of claim 17, further comprising calculating a well-being index score based on the wearable sensor data and user interactions with the general wellness curricula.

19. The method of claim 15, wherein the wearable sensor comprises an electroencephalogram (EEG) and the sensor data comprise EEG data.

20. The method of claim 1, wherein generating the well-being-related insights for the user is further based on a user-generated review.

21. The method of claim 1, wherein generating the well-being-related insights for the user is further based on user question and answer sessions.

22. The method of claim 1, wherein generating the well-being-related insights for the user is further based on user-generated check-ins.

23. The method of claim 1, further comprising awarding tokens to the user for providing a user generated narrative and providing a marketplace wherein the tokens are redeemable for items.

24. The method of claim 23, wherein the items comprise non-fungible tokens (NFTs).

25. The method of claim 1, further comprising providing a healthcare provider portal comprising the well-being-related insights for a healthcare provider associated with the user.

26. A computer-implemented system comprising a computing device comprising at least one processor and instructions executable to cause the at least one processor to perform operations comprising:
a) receiving media comprising an unstructured user-generated narrative;
b) applying algorithms to the unstructured user-generated narrative to extract semi-structured user context data;
c) applying a first machine learning model to classify one or more of sentiment, intent, habits, patterns, beliefs, and motivations from at least the user context data, wherein the first machine learning model comprises an unsupervised machine learning model,
d) applying a second machine learning model to identify a recommended next action from the user context data, wherein the second machine learning model comprises a supervised machine learning model;
e) generating a well-being-related insight for the user based on the user context data, and one or more of the sentiment, intent, habits, patterns, beliefs, motivations, and recommended next actions; and
f) generating an interactive representation, wherein the interactive representation is generated in real-time from the user-generated context narrative or well-being-related insight, and wherein the interactive representation comprises a virtual reality environment via visual, auditory, and tactile output devices, a music composition, or an image; and
g) fingerprinting the user-generated narrative, wherein the fingerprint authenticates the identity of the user.

27. One or more non-transitory computer-readable storage media encoded with instructions executable by at least one processor to provide a well-being application comprising:
a) a recording studio module configured to receive media comprising an unstructured user-generated narrative;
b) a user context extraction module configured to apply an algorithm to the unstructured user-generated narrative to extract semi-structured user context data;
c) a wisdom engine module configured to:
i. apply a first machine learning model to classify one or more of sentiment, intent, habits, patterns, beliefs, and motivations from the user context data, wherein the first machine learning model comprises an unsupervised machine learning model; and
ii. apply a second machine learning model to identify a recommended next action from the user context data, wherein the second machine learning model comprises a supervised machine learning model;
d) an insight generation module configured to generate a well-being-related insights for the user based on the user context data, and one or more of the sentiment, intent, habits, patterns, beliefs, motivations, and recommended next actions, and generate a virtual reality environment as an interactive representation of the well-being-related insights for the user, wherein the virtual reality environment produces real-time feedback to the user via visual, auditory, and tactile output devices; and
e) an authentication module configured to fingerprint a user-generated narrative, wherein the fingerprint authenticates the identity of a user.

28. The method of claim 1, wherein generating the virtual reality environment comprises producing real-time feedback to the user via visual, auditory, and tactile output devices within a machine that is occupied physically by the user.
